(19) United States
(12) Patent Application Publication
(10) Pub. No.: US 2022/0081676 A1
(43) Pub. Date: Mar. 17, 2022

(54) MICROFLUIDIC DEVICES FOR TATTOO PIGMENT SAFETY

(71) Applicant: EMULATE, INC., Boston, MA (US)

(72) Inventors: Lian Leng, Charlestown, MA (US); Justin Nguyen, Medford, MA (US); Norman Wen, West Roxbury, MA (US); Antonio Varone, West Roxbury, MA (US)

(21) Appl. No.: 17/484,255

(22) Filed: Sep. 24, 2021

Related U.S. Application Data
(63) Continuation of application No. PCT/US20/25508, filed on Mar. 27, 2020.
(60) Provisional application No. 62/867,486, filed on Jun. 27, 2019.
(60) Provisional application No. 62/824,743, filed on Mar. 27, 2019.

(51) Int. Cl. C12N 5/071; C12N 5/077; G01N 33/50

(52) U.S. Cl. CPC C12N 5/0629; C12N 5/0656; C12N 2533/70; C12N 2533/54; G01N 33/5044

(57) ABSTRACT

The present invention relates to devices including microfluidic devices, e.g. Skin on-Chip (Skin-Chip), for simulating a physiological response to agents and injury, including tattoo injury. In particular, a Skin-Chip is intended for use in replicating the interaction of tattoo ink with skin on a cellular level, including but not limited to mechanisms of wound healing following a tattoo gun and/or tattoo needle induced skin injury; ink particle effects such as pigment retention, pigment distribution and pigment clearance; inflammatory response to foreign particles, i.e. tattoo ink, etc. Further, effects of tattoo inks on simulated microfluidic skin is extended to determine effects of systemic ink exposure upon other organs through use of organ chips, e.g. liver-chips, kidney-chips, Lymph node-chips, etc. In some embodiments, safer ink formulations, e.g. less toxic ink particles, less toxic ink diluents, etc., are contemplated for development and use over currently available tattoo inks and diluents. Further contemplated is using a Tattooed Skin-Chip for developing rapid and non-toxic methods of removal of Tattoos in human skin.

### CROSS-REFERENCE TO RELATED APPLICATIONS

The present application is a continuation of, and claims benefit of, U.S. patent application Ser. No. 16/178,262 filed Nov. 1, 2018, now U.S. Pat. No. 10,626,446 issued Apr. 21, 2020, a continuation of U.S. patent application Ser. No. 14/264,758 filed on Apr. 29, 2014, now U.S. Pat. No. 10,160,995 issued Dec. 25, 2018, based on U.S. Provisional Patent Application No. 61/822,695 filed on May 13, 2013, now expired, all of which are incorporated herein by reference.

### FIELD OF THE DISCLOSURE

The present invention relates to microfluidic devices, e.g. Skin on-Chip (Skin-Chip), for simulating a physiological response to agents and injury, including tattoo injury. In particular, a Skin-Chip is intended for use in replicating the interaction of tattoo ink with skin on a cellular level, including but not limited to mechanisms of wound healing following a tattoo gun and/or tattoo needle induced skin injury; ink particle effects such as pigment retention, pigment distribution and pigment clearance; inflammatory response to foreign particles, i.e. tattoo ink, etc. Further, effects of tattoo inks on simulated microfluidic skin is extended to determine effects of systemic ink exposure upon other organs through use of organ chips, e.g. liver-chips, kidney-chips, Lymph node-chips, etc. In some embodiments, safer ink formulations, e.g. less toxic ink particles, less toxic ink diluents, etc., are contemplated for development and use over currently available tattoo inks and diluents. Further contemplated is using a Tattooed Skin-Chip for developing rapid and non-toxic methods of removal of Tattoos in human skin.

### BACKGROUND

Are Tattoos Safe for Humans to Wear?

Humans have tattooed themselves for millennia, motivated by reasons including as designs decorating their skin. Crusaders tattooed crosses on their bodies to ensure they'd go to heaven. Sailors inked their bodies to boast about where they'd traveled. The 61 tattoos on Ötzi, a 5,300-year-old mummy discovered in the Alps, were all located near his joints, leaving researchers to speculate whether these tattoos were part of an ancient arthritis treatment.

These days, the majority of the 120 million tattooed people worldwide inked themselves for fashion. In the United States there are an estimated 20,000 tattoo parlors or studios, contributing to a $1.35 billion industry which is growing in numbers of people. Further there is a growing $694 million tattoo removal market with 110,000 procedures performed by dermatologic surgeons in 2017. Average cost runs about $1,400, over 7 sessions. Tattoo removal is most often performed at medical spas, by dermatologists and other medical doctors.

European regulators and others are concerned that pigments used in the formulation of tattoo and permanent make-up inks are not produced for such purpose and do not undergo any risk assessment that takes into account their injection into the human body for long-term permanence. In the U.S. and Canada, policies that govern tattooing are also spotty. The FDA in the United States regulates the inks used for use in tattoos, but the actual practice of tattooing is regulated by local jurisdictions, including cities and counties. That means there is no standardized certification for those doing the tattooing or an overall governing body supervising the health and safety of tattoo parlors or even the inks.

Most consumers are aware of the infection risks, include hepatitis, staphylococcus, or viral warts, but few are aware of the chemical risks. While tattoos are commonplace, knowing the ingredients and provenance of the colorful cocktail injected beneath the skin is not. Tattoo inks contain a wide range of chemicals and heavy metals, including some that are potentially toxic. It's not widely known by the general public that the pigments found in tattoo inks can be repurposed from the textile, plastics, or the car paint industry.

Thus, tattoo artists also have concerns. While there are producers of ink considered acceptable for use on humans, some of the inks on the market weren't intended for tattooing people. In some cases, inks are placed in a fancy bottle, labeled with a dragon and ‘tattoo’.

In fact, the European Commission issued a report in 2016 highlighting the need for funding into research on tattoo ink toxicity and how tattoo inks break down in the body.

As of 2016, inks imported from the U.S. were responsible for two-thirds of the tattoo-related alerts sent to European authorities. One-quarter of problematic inks came from China, Japan, and some European countries

Therefore, there is a need for research on short-term and long-term health risks of tattooing and for harmonizing regulations controlling safety of tattoos and tattoo inks.

### SUMMARY OF THE INVENTION

The present invention relates to devices including microfluidic devices, e.g. Skin on-Chip (Skin-Chip), for simulating a physiological response to agents and injury, including tattoo injury. In particular, a Skin-Chip is intended for use in replicating the interaction of tattoo ink with skin on a cellular level, including but not limited to mechanisms of wound healing following a tattoo gun and/or tattoo needle induced skin injury; ink particle effects such as pigment retention, pigment distribution and pigment clearance; inflammatory response to foreign particles, i.e. tattoo ink, etc. Further, effects of tattoo inks on simulated microfluidic skin is extended to determine effects of systemic ink exposure upon other organs through use of organ chips, e.g. liver-chips, kidney-chips, Lymph node-chips, etc. In some embodiments, safer ink formulations, e.g. less toxic ink particles, less toxic ink diluents, etc., are contemplated for development and use over currently available tattoo inks and diluents. Further contemplated is using a Tattooed Skin-Chip for developing rapid and non-toxic methods of removal of Tattoos in human skin.

Moreover, methods described herein relate to providing and using a healthy Skin-Chip for determining responses to various types of skin injury, chemicals, etc., including skin remodeling in general. In some embodiments, Skin-chips have dynamic healthy skin (tissue) comprising a dermal region that is separated by a basement membrane from cycling keratinocytes, i.e., keratinocyte stem cells, that undergo division providing both renewable keratinocyte stem cells and progeny cells that undergo further cell division and differentiation for providing the stratified skin cell layers mimicking living skin tissue, including having upper cornified (keratinized) layers of dead cells. As keratinocytes progress in maturation to become dead cornified cells, they slowly die as they are pushed up into the upper layers of epidermis, especially under (air-liquid interface (ALI) culture conditions rather than liquid-liquid (L-L). These dead skin cell layers under ALI may slough off the top of the lower dying epidermal layer.

Epidermis in vivo has an abundance of folds, invaginations and specialized niches, where epidermal layers fold to form a cavity, tube or pouch as downgrowths of the epidermis into the dermis, in general “invaginations”. Invaginations in vivo may from any type of hair follicle, sebaceous gland, sweat gland, etc., found in the epidermal-dermal region of skin. One goal is to provide a healthy Skin-Chip having such invaginations forming epidermal structures found in vivo, such as hair follicle, sebaceous gland, sweat gland, rete ridges, dermal ridges, etc.

Thus, in some embodiments, a healthy Skin-Chip model is used for demonstrating tissue repair capabilities, in some embodiments full-thickness repair, in an in vitro human skin model, recapitulating physiological response to tattoo injury through a cascade of immune response, cell activation, migration and turn over, and ECM remodeling of the basement membrane, for one example.

Because skin is the largest organ of the body, providing a first line of defense against potentially damaging compounds, Skin-chips underwent testing showing their robustness by demonstrations of their use providing skin barrier, skin irritation, skin phototoxicity and photosensitization information.

However, there were problems with initial Skin-chips, such as those did not include a compound for delaying gel contraction, including a) dermal gel layers that contracted in the early days of the initial experimental timeline, especially when membrane stretch was induced, e.g. regular, cyclic, stretching that unfortunately dislodged cells from the epidermis (therefore stretch was not used in these experiments); b) spontaneous contractions of the gel-dermal region disrupting the continuity of the dermis and epidermis, in other words disrupting homogenous liquid-cell areas; c) variability of differentiation and maturation of keratinocytes of up to at least a 10 day difference or more between seeded chips using these same methods, i.e. a lack of repeatability (consistency). Further, because a window of consistent time of at least 2 weeks up to 21 days or more is necessary for obtaining useful toxicity study data, many of these chips were not useable.

Thus, during the development of the present inventions, at least 5 improvements were contemplated for use and testing for overcoming problems that resulted in providing a more robust Skin-Chip model, of which at least some of these inventive steps led to providing a full-thickness healthy Skin-Chip model under Air-Liquid Interface (ALI) conditions up to ten days or more earlier than without improvements described herein. Such improvements provided full-thickness skin under ALI for use with testing compounds over a longer time period, e.g. at least up to 14 days, up to 15 days, up to 16 days, up to 21 days, up to 24 days, or even 27 days or more over the time period of Skin-Chip model models not containing these improvements.

Thus, improvements for providing a more robust a healthy Skin-Chip include but are not limited to: 1) using cultured keratinocyte no later than passage 4 or 5 (for healthy appearing keratinocytes that can consistently reach confluency) for initially seeding microfluidic devices, in part for providing greater reproducibility of epidermal layers between Skin-chips; 2) using around 4-5 mg/ml collagen gels, e.g. for seeding fibroblasts into dermal regions of open top microfluidic devices, i.e. dermal gel; 3) adding a compound that can function to delay or inhibit gel contraction to collagen gels; 4) in some embodiments using an open top microfluidic device incorporating design changes including but not limited to having 6 anchors, each of which are larger in diameter than previous structural anchors used in much larger numbers in other embodiments of open top microfluidic devices; and 5) extending the period of ALI beyond 14 days.

As described herein, the present invention provides in one embodiment a method for seeding Skin-Chips where dermal fibroblast cells are seeded first (e.g. Day 0) within a gel matrix, followed by keratinocyte cells within 24 to 48 hours (e.g. Day 1), followed by the introduction of differentiation media for 24-72 hours, followed by exposure of the cells to ALI, in part for providing a differentiated skin chip for use faster than when using previous methods (e.g. providing a larger window of time for testing agents on the skin model). Prior methods often seeded dermal fibroblast cells 7 days after adding dermal gels, in part because lower concentrations of gels were used which took many days to solidify. Then, after the gel matrix solidified, the dermal cells were incorporated into and moved around inside of these low concentration gels. Further, these low concentrated gel matrixes would contact more than higher concentrations of gel matrixes.

In one embodiment, the present invention provides a method, comprising: a) providing i) a device (including but not limited to a microfluidic device) having a membrane, wherein said membrane has a first surface, ii) a population of dermal fibroblast cells, iii) a population of keratinocyte cells, iv) keratinocyte differentiation medium; and b) seeding said dermal fibroblast cells in a gel matrix, said gel matrix positioned on said first surface of said membrane; c) seeding said keratinocyte cells on top of said gel matrix after step b); d) culturing said keratinocyte cells in said keratinocyte differentiation medium under flow conditions; and e) culturing said cells under air-liquid-interface (ALI) conditions.

Keratinocyte cells often undergo rapid terminal differentiation in cell culture under maintenance conditions. It was discovered that using keratinocytes at passages later than 5 for seeding devices produced more inconsistent growth. Thus, in one embodiment, said keratinocytes cells, prior to seeding in said device at step c), were not passaged more than 5 times. In one embodiment, said keratinocytes cells, prior to seeding in said device at step c), were not passaged more than 4 times. In one embodiment, wherein prior to step d) said keratinocytes form a confluent layer. In one embodiment, wherein step e) is performed for up to 21 days. In one embodiment, wherein step e) is performed for up to 27 days. In one embodiment, wherein step c) is performed less than 72 hours after step b). In one embodiment, wherein step c) is performed 48 hours or less after step b). In one embodiment, wherein step c) is performed 24 hours or less after step b). In one embodiment, wherein step d) is performed for 48 hours or less, prior to step e). In one embodiment, wherein step d) is performed for 24 hours or less, prior to step e). In one embodiment, said device comprises a removable top, wherein said removable top is removed prior to step b). The removable top can be put back in place and then later removed for additional seeding. Typically, the top is maintained in place during cell culturing. In one embodiment, said culturing under air-liquid-interface conditions results in a epidermal layer positioned above a dermal layer. In one embodiment, said gel matrix is in contact with one or more structures that hold at least a portion of the gel in position for a time period (e.g. as compared to when the gel matrix is used without such structures). In one embodiment, said method further comprising the step of stretching the gel, the membrane or both. In one embodiment, said method further comprising f) exposing said epidermal layer to an agent. In one embodiment, said method further comprising f) wounding said epidermal layer.

In one embodiment, the present invention provides a method, comprising: a) providing i) a device (including but not limited to a microfluidic device) having a membrane, wherein said membrane has a first surface, ii) a population of dermal fibroblast cells, iii) a population of keratinocyte cells, iv) keratinocyte differentiation medium; and b) seeding said dermal fibroblast cells in a gel matrix, said gel matrix positioned on said first surface of said membrane and comprising collagen, said collagen in a concentration greater than 4 mg/ml (and preferably 5 mg/ml); c) seeding said keratinocyte cells on top of said gel matrix after step b); d) culturing said keratinocyte cells in said keratinocyte differentiation medium under flow conditions; and e) culturing said cells under an air-liquid-interface. In one embodiment, said collagen concentration is approximately 5 mg/ml. In one embodiment, said collagen comprises collagen I. In one embodiment, said keratinocytes cells, prior to seeding in said device at step c), were not passaged more than 4 times. In one embodiment, said method wherein prior to step d) said keratinocytes form a confluent layer (e.g. within 48 hours of seeding). In one embodiment, said method wherein step e) is performed for up to 21 days.

In one embodiment, said method wherein step e) is performed for up to 27 days. In one embodiment, said method wherein step c) is performed less than 72 hours after step b). In one embodiment, said method wherein step c) is performed 48 hours or less after step b). In one embodiment, said method wherein step c) is performed 24 hours or less after step b). In one embodiment, said method wherein step d) is performed for 48 hours or less, prior to step e). In one embodiment, said method wherein step d) is performed for 24 hours or less, prior to step e). In one embodiment, said device comprises a removable top, wherein said removable top is removed prior to step b). Typically, the top is maintained in place for culturing and removed in order to perform cell seeding. In one embodiment, said culturing under air-liquid-interface conditions results in a epidermal layer positioned above a dermal layer. In one embodiment, said gel matrix is in contact with one or more structures that hold at least a portion of the gel in position for a time period (as compared to the situation where no such structures are used). In one embodiment, said method, further comprising the step of stretching the gel, the membrane or both. In one embodiment, said method further comprising f) exposing said epidermal layer to an agent. In one embodiment, said method further comprising f) wounding said epidermal layer (as described herein).

In one embodiment, the present invention provides a composition comprising a gel matrix comprising a polymer, said polymer formed by the copolymerization of sucrose and epichlorohydrin (such as Ficoll™). However, other compounds that function to inhibit contraction of the gel are also contemplated (e.g. instead of Ficoll™) such as hyaluronic acid. In one embodiment, said gel matrix further comprises collagen. In one embodiment, said gel matrix further comprises cells. In one embodiment, said cells are fibroblasts. In one embodiment, said cells are keratinocytes. In one embodiment, said polymer formed by said copolymerization is a branched, hydrophilic polysaccharide which dissolves in aqueous solutions.

In one embodiment, the present invention provides a method, comprising seeding a population of cells in a gel matrix, said gel matrix comprising a polymer, said polymer formed by the copolymerization of sucrose and epichlorohydrin (Ficoll™). However, other compounds capable of inhibiting gel contraction are contemplated as well. In one embodiment, said gel matrix further comprises collagen. In one embodiment, said cells are fibroblasts. In one embodiment, said cells are keratinocytes. In one embodiment, said polymer formed by said copolymerization is a branched, hydrophilic polysaccharide which dissolves in aqueous solutions.

In one embodiment, the present invention provides a method, comprising: a) providing i) a device (including but not limited to a microfluidic device) having a membrane, wherein said membrane has a first surface, ii) a population of dermal fibroblast cells, iii) a population of keratinocyte cells, iv) keratinocyte differentiation medium; and b) seeding said dermal fibroblast cells in a gel matrix, said gel matrix positioned on said first surface of said membrane and comprising collagen and a polymer formed by the copolymerization of sucrose and epichlorohydrin (e.g. Ficoll™); c) seeding said keratinocyte cells on top of said gel matrix after step b); d) culturing said keratinocyte cells in said keratinocyte differentiation medium under flow conditions; and e) culturing said cells under an air-liquid-interface. In one embodiment, said polymer formed by said copolymerization is a branched, hydrophilic polysaccharide which dissolves in aqueous solutions. In one embodiment, said polymer inhibits the contraction of said gel matrix. In one embodiment, said polymer delays the contraction of said gel matrix for a period of time. In one embodiment, said polymer delays the contraction of said gel matrix for as much as three days, four days or even five days (when compared to conditions where no such polymer is added to the gel matrix). In one embodiment, said culturing under air-liquid-interface conditions results in a epidermal layer positioned above a dermal layer. In one embodiment, said method wherein at least a portion of the epidermal layer is embedded in said dermal layer (e.g. invaginations into the dermal layer). In one embodiment, said gel matrix is in contact with one or more structures that hold at least a portion of the gel in position for a time period (e.g. a period of days to weeks) when compared to conditions without such structures. In one embodiment, said method further comprising the step of stretching the gel, the membrane or both. In one embodiment, said method further comprising f) exposing said epidermal layer to an agent. In one embodiment, said method further comprising f) wounding said epidermal layer.

In one embodiment, the present invention provides a method, comprising: a) providing i) a device (including but not limited to a microfluidic device) having a membrane, wherein said membrane has a first surface, said first surface comprising a gel matrix and one or more structures that hold at least a portion of the gel in position for a time period (e.g. days to weeks), ii) a population of dermal fibroblast cells, iii) a population of keratinocyte cells, iv) keratinocyte differentiation medium; and b) seeding said dermal fibroblast cells into said gel matrix; c) seeding said keratinocyte cells on top of said gel matrix after step b); d) culturing said keratinocyte cells in said keratinocyte differentiation medium under flow conditions; and e) culturing said cells under air-liquid-interface conditions. In one embodiment, said keratinocytes cells, prior to seeding in said device at step c), were not passaged more than 4 times. In one embodiment of said method, prior to step d), said keratinocytes form a confluent layer (e.g. a layer without visible gaps). In one embodiment, said structures are posts surrounding said gel matrix (e.g. 4-8 posts, and more preferably 6 posts).

Surprisingly with the addition of Ficoll in the dermal gel, as demonstrated, herein, there was better keratinocyte (cells) (KC) attachment to the dermal region. In addition, Ficoll also appeared to induce more epidermal-dermal invaginations when compared to not using Ficoll, see FIG. 9 Skin-Chip without Ficoll. These invaginations are surprising and more vivo like (biopsies of healthy human skin have may such invaginations). By contrast, epithelium created in transwell devices produced epidermis having few if any observed invaginations.

In some embodiments, the use of Ficoll added to gels prior to placement within chip chambers, delays onset of spontaneous gel contractions for up to 5 days or more. While not intended to limit the invention in any manner, in one embodiment the Ficoll added to gels is a mixture of Ficoll particles 70 MW and 400 MW.

The present invention provides a method, comprising: a) providing a microfluidic device comprising at least one layer of living keratinocyte cells, and a wounding device for creating a wound; and b) wounding said cells with said wounding device whereby said at least one layer of living keratinocyte cells is disrupted. In one embodiment, said wounding device is selected from the group consisting of a solid needle; a hollow needle; a syringe needle, microneedles, a tattoo needle, a tattoo gun, a wire brush, a scalpel, a dermabrasion device, and a freezing solution spray device. In one embodiment, said microfluidic device comprises i) a chamber, said chamber comprising a circular lumen, said lumen comprising ii) a gel matrix comprising fibroblasts and said keratinocyte layer, said gel matrix positioned above iii) a porous membrane, said membrane positioned above one or more iv) fluidic channels. In one embodiment, the method further comprises a step between a) and c), said step comprising applying a test substance to said keratinocyte layer. In one embodiment, said test substance comprises a compound for enhancing would healing. In one embodiment, said test substance comprises a compound for preventing wound healing. In one embodiment, said test substance comprises a compound for inhibiting wounding. In one embodiment, said test substance is selected from group consisting of TiO₂ particles, pigment particles, metal particles, carbon black particles, and tattoo ink. In one embodiment, said test substance is selected from the group consisting of diluents, glycerin, propylene glycol, witch hazel, a steol, syntran, planterin, and alcohol. In one embodiment, said test substance is selected from the group consisting of a freezing compound, a cytotoxic compound, an irritant compound, a sensitizer compound, a corrosive compound, and a phototoxic compound. In one embodiment, said test substance is selected from the group consisting of fluorescent particles, cell tracker particles, and fluorescent microbeads. In one embodiment, said wounding device is used to apply said test substance. In one embodiment, microfluidic device further comprises a microfluidic channel and said test substance is introduced into said microfluidic channel. In one embodiment, fibroblasts are within the gel matrix and the keratinocyte layer is on top of the gel matrix. In one embodiment, keratinocytes comprise more than one layer on top of the gel matrix. In one embodiment, at least one layer is keratinized. In one embodiment, the method further comprises c) measuring the amount of time between when said wounding occurs and when said keratinocyte layer is no longer disrupted. In one embodiment, said keratinocytes are human foreskin keratinocytes. In one embodiment, said gel matrix comprises collagen. In one embodiment, said gel matrix is between 0.2 and 6 mm in thickness. In one embodiment, said microfluidic device further comprises endothelial cell. In one embodiment, said endothelial cells are selected from the group consisting of primary cells; primary cells as small vessel human dermal microvascular endothelial cells; human umbilical vein endothelial cells; and bone marrow-derived endothelial progenitor cells.

The present invention provides a method, introducing particles into cells, comprising: a) providing i) a microfluidic device comprising at least one layer of living keratinocyte cells, ii) a device for depositing pigment particles, and iii) a plurality of pigment particles in a pigment diluent solution; b) introducing said pigment particles into said cells with said device under conditions wherein at least a portion of said keratinocyte layer is disrupted. In one embodiment, said device is a tattoo device. In one embodiment, said after step b), said particles are disposed within the keratinocyte layer. In one embodiment, said after step b), said particles are disposed below the keratinocyte layer. In one embodiment, the method further comprises c) measuring the amount of time between when i) said pigment solution is deposited and ii) when said keratinocyte layer is no longer disrupted. In one embodiment, said pigment particle is selected from group consisting of TiO₂ particles, metal particles, pigment particles, carbon black particles, fluorescent particles, cell tracker particles, fluorescent microbeads and tattoo ink. In one embodiment, said diluent is selected from group consisting of tattoo ink diluent, alcohol, glycerin, propylene glycol, witch hazel, a steol, syntran, and planterin. In one embodiment, said microfluidic device further comprises i) a chamber, said chamber comprising a circular lumen, said lumen comprising ii) a gel matrix comprising fibroblasts and said at least one layer of keratinocytes, said gel matrix positioned above iii) a porous membrane, said membrane positioned above one or more iv) fluidic channels. In one embodiment, the method further comprises a step between a) and c), providing a test substance and applying said test substance to said keratinocytes. In one embodiment, said test substance is selected from group consisting of alcohol, lidocaine, and antibiotic compounds.

The present invention provides a method, comprising: a) providing a microfluidic device comprising at least one layer of keratinocytes, said keratinocytes comprising pigment particles; and b) treating said particles with a device or compound whereby said pigment particles are disrupted. In one embodiment, said microfluidic device further comprises i) a chamber, said chamber comprising a circular lumen, said lumen comprising ii) a gel matrix comprising fibroblasts and said at least one layer of keratinocytes, said gel matrix positioned above iii) a porous membrane, said membrane positioned above one or more iv) fluidic channels. In one embodiment, said particles are treated with a compound that causes a cellular response in said fibroblasts, keratinocytes or both. In one embodiment, the method further comprises detecting said cellular response. In one embodiment, the method further comprises detecting when said cellular response is no longer present. In one embodiment, the method further comprises c) measuring the amount of time between when said pigment particles are disrupted and when said pigment particles are no longer visible. In one embodiment, said treating is with a device selected from group consisting of a laser device and an intense pulsed light therapy (IPL) device. In one embodiment, said laser device is selected from group consisting of a Q-switched Nd: YAG laser, Q-switched Alexandrite laser and a Q-switched Ruby laser.

The present invention provides a method, comprising: a) providing a microfluidic device comprising at least one layer of living keratinocyte cells, and a test compound; b) applying said test compound to said layer of cells whereby said living keratinocyte cells are disrupted; and c) determining the length of recovery time of said disrupted layer of living keratinocyte cells. In one embodiment, said test compound is selected from the group consisting of citric acid, lactic acid and glycolic acid. In one embodiment, said disrupted living keratinocyte cells results in cell death. In one embodiment, said disrupted living keratinocyte cells results in reduced metabolism of said cells. In one embodiment, said method further comprises a second test compound, wherein administration of said second test compound reduces said length of recovery time. In one embodiment, said second test compound is an acid neutralizing solution. In one embodiment, said second test compound comprises a retinoid compound. In one embodiment, said microfluidic device comprising at least one layer of living keratinocyte cells was treated with a pre-treatment compound before applying said test compound.

### Definitions

The term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

“Channels” are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

As used herein, the phrases “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid reservoir. Two components may be coupled to each other even though they are not in direct contact with each other. For example, two components may be coupled to each other through an intermediate component (e.g. tubing or other conduit).

As used herein, the term “biopsy” refers to a sample of the tissue that is removed from a body.

As used herein, “full-thickness” in reference to an artificial skin, e.g. as provided in a full-thickness Skin-Chip, refers to a stratified epidermis including a basement membrane, e.g. identifiable by detection of extracellular matrix proteins including but not limited to collagen IV, Laminin 5, etc., stratum basal layer, Keratin 14, Stratum spinosum: Keratin 10, Stratum granulosum: Filaggrin, Stratum corneum: Involucrin, Loricrin.

### BRIEF DESCRIPTION OF THE DRAWINGS

Exemplary embodiments are illustrated in referenced figures. It is intended that the embodiments and figures disclosed herein to be considered illustrative rather than restrictive.

The file of this patent contains at least one drawing executed in color. Copies of this patent with color drawings will be provided by the Patent and Trademark Office upon request and payment of the necessary fee.

FIG. 1A-B shows an illustrative schematics for embodiments of wound healing, e.g. by tattoo needles or tattoo guns.

FIG. 1A illustrates epidermal and dermal injury, followed by epidermal and dermal healing. Tattoo pigments are retained within the dermal matrix and dermal fibroblasts. Pigments may also be well as transported downstream, with or without immune cells through the lymphatic and/or circulatory system thereby causing systemic effects.

FIG. 1B Illustrates how agents distribute in the body from the skin to downstream lymphatic and/or circulatory system fluidic connections thereby potentially causing systemic effects, e.g. effects upon lymph node cell activation (or suppression), effects upon liver, kidney, lung brain, etc. Numbers refer to exemplary sizes of cells/particles. Exemplary effects upon skin include but are not limited to: cytotoxicity; phototoxicity; sensitization/allergen; corrosivity/irritancy; carcinogenicity, etc. exemplary systemic effects leading to effects upon other organs include but are not limited to: cytotoxicity; carcinogenicity; effect on metabolism/organ function, etc. in some embodiments, downstream microfluidic devices simulating organs, such as lymph node-chips, liver-chips, kidney-chips, etc. are used for testing effluent from treated Skin-chips.

In some embodiments, organ chips are tested directly, i.e. individually. In some embodiments, organ chips are fluidically connected downstream of a treated Skin-Chip.

FIG. 2A-B illustrates one embodiment of a stretchable open top chip device.

FIG. 2A bottom structure with a spiral microchannel with an inlet well and an outlet well.

FIG. 2B A top view of a spiral microchannel configured with a circular vacuum chamber.

FIG. 3A illustrates an exploded view of one embodiment of a stretchable open top chip device (3000) demonstrating the layering of a fluidic top, top structure and bottom structure.

FIG. 3B illustrates a cut-away view of one embodiment of a stretchable open top chip device (3100) showing the regional placement of assay cells (e.g., epithelial cells, dermal cells and/or vascular cells), further demonstrating a lumen comprising an epithelial compartment and a stromal compartment in addition to a vascular compartment.

FIG. 3C illustrates a fully assembled view of one embodiment of a stretchable open top chip device.

FIG. 3D illustrate one embodiment of an exploded view of a stretchable open top chip device.

FIG. 4A-B shows one embodiment of an Open Top Chip.

FIG. 4A shows one embodiment of an assembled chip, showing the open-top chambers above, and separated by a membrane from, the lower channel fluidics.

FIG. 4B shows exemplary exploded view of an open top chip comprising two systems on one chip, wherein the membrane is highlighted in order to illustrate the relationship of the assembled components.

FIG. 5A-B shows exemplary schematic diagram of an open top chip comprising two adjacent and parallel microchannels.

FIG. 5A and FIG. 5B respectively illustrate an assembled isometric view and an exploded view of a tall channel stretchable open top chip device.

FIG. 6 illustrates a top assembled view of one embodiment of a stretchable open-top microfluidic chip comprising a fluidic cover and a single channel.

FIG. 7A-B illustrates a cross-sectional view of one embodiment of a stretchable open top microfluidic chip along plane A of FIG. 6. Shown is a top assembled view of one embodiment of a stretchable open-top microfluidic chip comprising a fluidic cover and a single channel. FIG. 7A Illustrates a fluidic cover in a closed position. FIG. 7B Illustrates a fluidic cover in an open position.

FIG. 8A-B illustrates a cross-sectional view of a one embodiment of a stretchable open top microfluidic chip along plane A of FIG. 7.

FIG. 8A Illustrates a fluidic cover in a closed position.

FIG. 8B Illustrates a fluidic cover in an open position.

FIG. 9 shows exemplary comparative images of skin in a biopsy (left panel) and in a Skin-chip (right panel), created without using Ficoll™, stained using H&E. An exemplary schematic of one embodiment of an open-top Skin Chip is illustrated below the microscopic images.

FIG. 10 illustrates an exemplary schematic of a microfluidic chip comprising skin, wounded by a tattoo needle, followed by healing, pigment uptake, clearance. wound healing cascade and pigment retention in Skin-Chip assessed using Tattoo pigments and fluorescent beads. Tattoo on Skin-Chip/Top View.

FIG. 11 shows exemplary dermal cells (fibroblasts) uptake ink and fluorescent beads in plate culture (upper panels) and in a Skin-chip, lower panels. Fibroblasts contribute to tattoo permanence by engulfing foreign particles. Fibroblast actively uptake and retain tattoo pigments and fluorescent particles of all size ranges.

FIG. 12 shows exemplary immune cell activation within wounded region as an in vitro immune response: Phagocytosis of Foreign Particles. Schematic, left panel, illustrating ink particles in a dermal region (light pink) through an epidermal tattoo wound (clear) in the epidermis (dark pink). Microscopic image middle showing part of a dermal region in a Skin-Chip where blue arrows point to CD80+ dark stained immune cells, e.g. a MV4-11 macrophage cell line (e.g. (ATCC® CRL-9591™) added to the dermal region, that cluster within tattooed region via inflammatory response (CD80+showing an activated pro-inflammatory phenotype—M1).

FIG. 13 shows exemplary immune cell (macrophage) activation within a tattooed area of a Skin-Chip. A range of fluorescent particles are taken up by cells. Size range vs. color?

FIG. 14A shows an exemplary full-thickness healthy Skin-Chip used for recreating human in vivo skin function, e.g. wound healing.

FIG. 14B shows exemplary microscopic images of H&E stained cross sections of one embodiment of a Skin-Chip showing wound healing following puncture wounds. A full-thickness Skin-Chip is able to heal through epidermal and dermal remodeling of wound site. Day 0—tattoo injury. 4 Days post-tattoo wound closure.

FIG. 15 shows exemplary Tattoo on Skin-Chip: pigment shedding through the epidermis. Healthy 2 days post-tattoo 6 days post-tattoo epidermal and dermal remodeling during wound healing show trapped pigment within epidermis, which become isolated and pushed upwards as the epidermal cells go through their cycle of stratification towards to upper most layer of the skin (normal skin cycle=14 days).

FIG. 16A-B shows exemplary epidermis and dermis remodeling during wound healing.

FIG. 16A shows exemplary micrographs of epidermis and dermis 2 Days Post-Tattoo. Blue arrow points to pigment.

FIG. 16B shows exemplary immunofluorescent micrographs demonstrating expression of key wound healing proteins Keratin 17 (pink) and Fibronectin (green). Nuclei colored blue. Epidermal and dermal wound closure observed by keratinocyte migration from the wound edge and dermal contraction.

FIG. 17 shows exemplary schematic illustrations of a conventional transwell device, left, having skin (dermis and epidermis)-orange, media-pink, separated by a nonstretchable porous membrane-blue dotted line, fluid was changed using a pipette; and a hybrid transwell chip device for mimicking conditions in a microfluidic chip, however with the absences of stretching and fluid flow, with a surface area smaller than a conventional transwell, right. Skin (dermis and epidermis)-orange, media-pink, separated by a stretchable porous membrane-blue dotted line. Fluid was changed using a pipette. In some embodiments, a microfluidic chip was used as a HTW, however again there was no stretch or continuous fluid flow, microchannels may be briefly flushed using microfluidic systems to provide fresh media.

FIG. 18 shows an exemplary microscopic image demonstrating full-thickness skin that forms within a static chip platform, inset shows a higher magnification. Immunofluorescent image demonstrates exemplary biomarkers for keratinocyte layers, e.g. K14 (green) and loricrin (red) and ECM, i.e. Collagen IV.

FIG. 19 shows exemplary Toxicity in the Skin-Chip: TiO₂ Tattooed on Static Skin-Chip Model. Metabolic activity (Presto Blue). * p value<0.001. Tattooed on D4 at ALI, N=4 per condition Hybrid Transwells.

Dose-dependent response 24 h post-tattoo observed by a decrease in viability with increasing TiO₂ concentration. Skin recovery observed over 7 days, except at highest dose leading to tissue necrosis.

FIG. 20 shows exemplary wound repair biomarkers MMP-9 and TGF-B after tattooing with TiO2, Blue and Red pigments. Hybrid Transwells.

MMP-9 was expressed after wounding during keratinocyte migration towards the wound edge, and by dermal fibroblasts through ECM degradation and invasion. Presence of tattoo pigments impedes production of MMP-9 involved in wound repair, slowing down the repair process. TGF-B inflammatory cytokine controls procollagen expression. Tattooed on D4 at ALI N=3 per condition Hybrid Transwells.

FIG. 21 shows exemplary Toxicity of Blue15 Tattooed on Static Skin Model. Metabolic Activity (Presto Blue) of Blue15 Pigment. Tattooed on D4 at ALI. N=4 per condition Hybrid Transwells. Control; Wounded; Percent viability from Control (%). Blue15 pigment does not cause observable changes in tissue viability over 6 days post-tattoo.

FIG. 22 shows exemplary Metabolic Activity (Presto Blue) Red122 Pigment. Concentration-dependent response 24 h post-tattoo observed by a decrease in viability with increasing Red122 concentration. Skin recovery observed over 8 days post-tattoo Tattooed on D4 at ALI N=4 per condition Hybrid Transwells.

FIG. 23 shows exemplary schematic timelines for providing full-thickness epidermal layers in Hybrid Transwells, along with an exemplary read out points. Lower timeline shows exemplary compound testing, e.g. permeability compounds Testosterone and Caffeine. Tissue robustness evaluated by determining the ET50 value following topical exposure to 1% TritonX-100. Static Skin model resulted in ET50 value around 11 h, compared to skin equivalent models on the market (e.g. MatTek—7 h).

FIG. 24A-C-shows exemplary micrographs comparing FIG. 24A Skin chip (static) D18 in culture (D14 at ALI), FIG. 24B EpiDerm200 from MatTek. FIG. 24C shows that ET50 for one embodiment of a Skin-Chip is better at ET50=11 hours vs. around 7 hours with EpiDerm200 from MatTek.

FIG. 25 shows an exemplary Caffeine Permeability. D14 ALI N=3 per condition Hybrid Transwells.

FIG. 26 shows an exemplary Skin Irritation—Release of Associated Cytokines. Topical exposure to a known skin irritant, Triton X-100, shows increase in expression of inflammatory cytokine IL-1alpha. IL-18, expressed following skin sensitization, shows no significant increase after exposure to Triton X-100.

D11 ALI. N=3 per condition Hybrid Transwells.

FIG. 27 shows an exemplary Safety Assessment of Red Tattoo Pigment—Phototoxicity Assay (Viability). Red tattoo pigments known to cause some level of toxicity following sun exposure.

Static Skin models were exposed to minimal dose of UVA which does not cause tissue damage (5 J/cm²).
Samples tattooed with 35% w/v of Red122 pigment showed some phototoxicity with a decrease in viability to 85% from healthy.
D12 ALI (D8 post-tattoo) N=4 per condition. Hybrid Transwells.

FIG. 28 shows an exemplary Skin tattooed with 35% w/v of Red pigment showed an increased secretion of inflammatory cytokine, but no effect as a skin sensitizer. Following UVA exposure, tattooed samples showed increase in both cytokines, indicating that Red is both phototoxic and a skin sensitizer under UVA. UVA on D11 ALI (D7 post-tattoo) N=3 per condition. Hybrid Transwells.

FIG. 29 shows an exemplary Phototoxicity to TiO₂ and Blue15—Cytokines Release. * P value<0.0001. UVA on D18 ALI (D14 post-tattoo). N=3 per condition. Hybrid Transwells.

FIG. 30 shows one embodiment of an examplary improved open top device having a 6-pillar chamber. Pillars may be from 4 to 12, however in part due to mechanical difficulties during pipetting gels and subsequent problems of gel contraction, a 6-pillar design provided optimal conditions. Pillar Dimensions: 750 um×1000 um at the base (tapers up); 1.5 mm tall Chamber height 3 mm with a 400 um×400 μm Bottom channel. shows an exemplary schematic illustration of methods for providing a full-thickness Skin-Chip comprising new design features, compatible with a biological incubator and Zoe.

FIG. 31 shows one embodiment of an examplary improved open top device having Open-Top Chip design changes to both basal and apical compartments.
Addition of gel anchoring pillars within the open-top
Modification of bottom channel from spiral configuration

Resulting from these improvements:
Gel stability (mitigate contraction and delamination)
Minimize bubble formation within bottom channel

FIG. 32 shows one embodiment of an improved open top device. Lower panel shows a photographic image of an improved Skin-Chip comprising new design features resting on a gloved finger (purple).

FIG. 33 shows exemplary embodiments of shorter timelines for use with microfluidic Skin-chips, e.g. using a cell culture device (e.g., Zoe). showing an exemplary bright filed image of keratinocytes sultures prior to seeding in Skin-Chips.

FIG. 34 A-D shows exemplary microscopic imges of cells after seeding. FIG. 34A) Keratinocyte seeding (D0), FIG. 34B) Slight epidermal contraction leading to holes, significantly more noticeable when cultured on stiff gels (bovine collagen type I≥5 mg/mL) (D1), FIG. 34C) Differentiation medium (D3), FIG. 34D) Air-liquid interface ALI (D4).

FIG. 35 shows exemplary microscopic representative image of a confluent epidermal monolayer on top of a dermal layer (D3 post-keratinocyte seeding).

FIG. 36 shows exemplary microscopic images of H&E stained cross sections of fixed, e.g. formalin fixed, epidermal and dermal regions formed after D3 ALI, D7 ALI and D11 ALI in microfluidic Skin-Chips, wherein Ficoll™ was added to the dermal gel, as described herein. Arrows point to surprising epidermal-dermal invaginations, where the epidermis appears to be embedded (integrated) within the dermal region. Thse types of invaginations were not observed in cross-sections of skin from devices without Ficoll™, see for one example, FIG. 9.

Additional observations: ALI was maintained over 21 days, up to at least 27 days; however after around Day 14 ALI a flush cycle was needed to clear flooding every 3-4 days due to some gel contraction around pillars (leading to slight flooding). Bubble occurrences in the bottom channel was minimal, e.g. 2/12 chips once about every 7 days.

FIG. 37 shows exemplary schematic embodiments for testing biodistribution of tattoo pigments or other particulate test compounds between the skin—liver and kidney.

FIG. 37 shows exemplary Assessing Tattoo Pigment Safety using Organ-Chips: Biodistribution of Tattoo Pigment. TiO₂ topical treatment of burn wounds.

FIG. 38 shows exemplary schematic embodiments for testing biodistribution of tattoo pigments or other particulate test compounds between the skin—lymph node—liver—kidney and other organs, such as brain and intestine.

### S-1 Chip

FIG. 39A-C illustrates embodiments of an exemplary S1 microfluidic device which may find use with the present invention.

FIG. 39A Illustrates a perspective view of a microfluidic device with microfluidic channels in accordance with an embodiment.

FIG. 39B Illustrates an exploded view of the device 200 in accordance with an embodiment, showing a microfluidic channel in a top piece 207 and a microfluidic channel in a bottom piece, separated by a membrane 208.

FIG. 39C shows cells in relation to device parts in a closed top chip, e.g. upper microchannel (1-blue); lower microchannel (2-red) and optional vacuum chamber (6). 1. Options include a liquid microchannel; air-liquid microchannel (upper); 2. Vascular channel (lower); 3. parenchymal cells, including but not limited to epithelial cells/tissue (e.g. liver, kidney, lung), other types of cells, reticular cells (e.g. lymph node), neuronal cells, pericytes astrocytes (e.g. brain); 4. Simulated capillaries (e.g. endothelial cells matching or compatible with the cells in the upper chamber); 5. Membrane, stretchable; and 6. Vacuum Channels. Arrows represent direction of fluid flow.

### Liver-Chip

FIG. 40A shows an exemplary schematic embodiment of a Liver-Chip for assessing pigment toxicity in the Liver-Chip. Left panel, J Clin Invest. 2007; 117(3):539-548.

FIG. 40B shows exemplary schematic embodiments of a Liver-Chip as a microfluidic device which may find use with the present invention.

FIG. 41 shows one exemplary schematic embodiment of a quad Liver-Chip 1-8.

FIG. 42 shows exemplary readouts for assessing pigment toxicity in the Liver-Chip. Readouts include but are not limited to albumin secretion and transporter studies, e.g. CLF as a BSEP transporter substrate for showing bile acid accumulation.

FIG. 43 shows an exemplary timeline and experimental variables.

FIG. 44 shows exemplary Liver Damage at Day 6 Post Exposure to TiO₂: LDH Leakage & Albumin Secretion.

Liver Hepatocytes left LDH Leakage chart, Endothelial Cells (LSEC), LDH Leakage in Liver Endothelial Cells (LSEC) shown in the right LDH chart. Injury to liver function is indicated by increases in albumin Secretion.
No significant cell damage (LDH leakage) observed following exposure to TiO₂.
No significant change in cell function (albumin secretion) following exposure to TiO₂.
Other functional changes or mechanistic routes may be detectable through additional assays

FIG. 45 shows one exemplary Hepatocyte Cell Morphology (upper 6 panels) and nonparenchymal cell (NPC) Morphology (lower 6 panels) after 4 Days of Treatment with a range of concentrations of Blue15 comparing to staurosporine (5 microM) as a positive control showing hepatocyte damage. Black arrow points to gaps in cell coverage indicated a loss of cells.

FIG. 46 shows one exemplary embodiment of a Liver-Chip demonstrating albumin secreted over time measured in effluent from the top channel. A range of concentrations of Blue15 was tested comparing to staurosporine (5 microM) as a positive control showing a loss of secreted albumin.

FIG. 47 shows one exemplary Hepatocyte Cell Morphology (upper 6 panels) and nonparenchymal cell (NPC) Morphology (lower 6 panels) after 4 Days of Treatment with a range of concentrations of Red122 comparing to staurosporine (5 microM) as a positive control showing hepatocyte damage. Red arrow points to gaps in cell coverage indicated a loss of cells.

FIG. 48 shows one exemplary embodiment of a Liver-Chip demonstrating albumin secreted over time measured in effluent from the top channel. A range of concentrations of Red122 was tested comparing to staurosporine (5 microM) as a positive control showing a loss of secreted albumin.

FIG. 49 shows one exemplary embodiment of a Liver-Chip demonstrating effects of Red122 & Blue15 on Liver Function via ATP Synthesis as measured from the effluent collected from the bottom channels. A range of concentrations of Red122 (left) & Blue15 (right) were tested comparing to staurosporine (5 microM) as a positive control showing a loss of ATP synthesis.

S-1 Chip with a Double Membrane

FIG. 50 illustrates one embodiment of a double membrane microfluidic device that may find use with the present invention. In one embodiment, such a device may be used as a Lymph Node-Chip.

FIG. 50A illustrates a perspective view of an organ mimic device in accordance with an embodiment that contains three parallel microchannels separated by two porous membranes.

FIG. 50B illustrates a perspective view of an organ mimic device in accordance with an embodiment.

FIG. 50C illustrates a device containing three channels as described in FIG. 50A.

### Lymph Node-Chip

FIG. 51 shows one exemplary embodiment of a Lymph Node-Chip for assessing pigment toxicity, as one embodiment of a microfluidic device that may find use with the present invention. Micrograph shows a close up image of Jurkat cells and microfabricated traps that comprise the Lymph-Node Organ Chip.

### Renal-Kidney Proximal Tubule-Chip

FIG. 52A shows an exemplary embodiment of a Renal-Kidney Proximal Tubule-Chip, as one embodiment of a microfluidic device, for assessing pigment toxicity using exemplary biomarkers as shown.

FIG. 52B shows exemplary embodiments of a Renal-Kidney Proximal Tubule-Chip for assessing pigment toxicity and demonstrating types of exemplary readouts of toxicity. Gentamicin treatment is used for inducing damage as a positive control compared to controls without Gentamicin. Such readouts include but not limited to biomarkers, morphology differences and physiological differences, such as demonstrated by changes in observed morphology, LDH activity, Caspase activity, NAG activity and Reactive Oxygen Species (ROS) activity.

FIG. 53 shows exemplary embodiments of methods for providing Renal-Kidney Proximal Tubule-Chip experimental timelines for using Renal-Kidney Proximal Tubule-Chip when assessing compound toxicity, e.g. pigment toxicity, dye treatment, tattoo inks, in addition to ink diluent or other nonpigment compounds used or found in tattoo inks, toxic compounds used or found in tattoo inks, cosmetic compounds.

FIG. 54 shows exemplary Kidney Proximal Tubule-Chip at Day 6 Post Exposure to TiO₂ (0.003%, 0.05% and 0.24% TD): Kidney Epithelial Cells Morphology. Severe morphological changes are observed following 30 microM Cisplatin treatment (positive control). No significant morphological change observed with TiO₂ exposure. Blue arrows point to examples of cell detachment in Cisplatin treated chips. Pink arrows point to examples of pigment aggregates in TiO₂ treated chips.

FIG. 55 shows exemplary microscopic images of Kidney Proximal Tubule-Chip morphology on Day 6 Post Exposure to TiO₂. An exemplary microscopic image of endothelial cells after they were treated with 0.24% TD TiO2 nanoparticles. Nanoparticles (black) can be seen internalized in the endothelial cells surrounding the nucleus (oval and circular clear areas).

FIG. 56 shows exemplary Kidney Proximal Tubule-Chip: Assessment of Toxicity via Morphological Score showing a poor cell morphology rating on Day 6 Post Exposure to TiO₂. Observed trend in decline of endothelial morphological quality with increasing concentration of TiO₂. The epithelial layer was not evaluated due to pigments covering the cell monolayer. Morphological Score provides a rating of the quality of cell morphology assessed via morphological scoring. High score correlates with poor cell morphology.

FIG. 57 shows exemplary Kidney Proximal Tubule-Chip epithelial cell damage at Day 6 Post Exposure to TiO2: LDH Leakage & NAG Activity.

No significant cell damage (LDH leakage) observed following exposure to TiO₂. NAG activity showed no toxicity at lower dose, but some effect at a higher concentration of 0.24% TD. Additional assays may detect other mechanistic levels of kidney toxicity.

FIG. 58 shows exemplary Red122 and Blue15 effects on morphology and growth.

FIG. 59 shows exemplary Red122 and Blue15

FIG. 60 shows exemplary Red122 and Blue15: Effect on Kidney Toxicity via LDH Release and Caspase induction.

FIG. 61 shows exemplary Kidney Proximal Tubule-Chip pigment ROS.

FIG. 62 shows exemplary Kidney Proximal Tubule-Chip pigment caspase and ALP.

FIG. 63 illustrates exemplary schematics of some embodiments of a BBB-chip/Brain-chip, as embodiments of a microfluidic device that may find use with the present invention.

FIG. 64A shows exemplary evaluation of a compound's toxicity to epidermal cells in a Skin-Chip (microfluidic OT-chip), after a 15-minute exposure, by DRAQ7™ staining. Citric acid (0.6M), lactic acid and glycolic acid were each tested on a Skin-Chip. At the concentrations added, each of the three acids showed statistically significant death of skin cells in the chip with glycolic acid showing the highest cell death, dead cells/mm². Insert shows highly florescent dead cells.

FIG. 64B shows exemplary evaluation of a compound's effect upon metabolic activity measured by PrestoBlue™, as a percentage of duplicate control-untreated skin-chips (microfluidic OT-chip). On Day 0 (D0), each of the three acids shows a reduction in activity compared to control (100%). However glycolic acid shows a statistically significant reduction in activity compared to the activity of citric acid (0.6M) and lactic acid. By Day 1 (D1), remaining cells treated with each one of the three acids shows a higher metabolic activity as they are recovering, than on D0, while citric acid metabolic activity recovery was statistically significantly higher than lactic acid and glycolic acid exposed cells.

FIG. 65 shows exemplary 4 days post-tattoo wound closure.

### DESCRIPTION OF INVENTION

The present invention relates to devices including microfluidic devices, e.g. Skin on-Chip (Skin-Chip), for simulating a physiological response to an agent or injury, including tattoo injury. In particular, a Skin-Chip is intended for use in replicating the interaction of tattoo ink with skin on a cellular level, including but not limited to mechanisms of wound healing following a tattoo gun and/or tattoo needle induced skin injury; ink particle effects such as pigment retention, pigment distribution and pigment clearance; inflammatory response to foreign particles, i.e. tattoo ink, etc. Further, effects of tattoo inks on simulated microfluidic skin is extended to determine effects of systemic ink exposure upon other organs through use of organ chips, e.g. liver-chips, kidney-chips, Lymph node-chips, etc. In some embodiments, safer ink formulations, e.g. less toxic ink particles, less toxic ink diluents, etc., are contemplated for development and use over currently available tattoo inks and diluents. Further contemplated is using a Tattooed Skin-Chip for developing rapid and non-toxic methods of removal of Tattoos in human skin.

### I. Organs-On-Chip: Human Emulation for Tattoo Pigment Safety: Simulating Tattooing on Skin-Chips.

Obtaining a skin tattoo in vivo includes wounding of skin as tattoo ink is injected, wound healing, pigment uptake in skin, and sooner or later involves pigment clearance from the skin. Following epidermal healing from this wounding, tattoo pigments are retained within the dermal matrix and dermal fibroblasts, as well as transported downstream via immune cells and through the circulatory system. Moreover, because many people decide to remove a tattoo, pigment removal induced damage of skin may also be involved. As described herein, the inventors have a goal for understanding tattoo permanence and pigment clearance using a microfluidic Skin-Chip.

Furthermore, because of the lack of regulation of the tattooing process and ink formulations, there is a need for determining ink effects in skin including: movement of inks through the skin and beyond; impact of internal inks on human health as tattoo safety; including bioavailability and systemic exposure of tattoo inks.

TABLE 1A
Skin Tattoos: Through the Skin and
Beyond, Impact on Human Health.

| Exemplary Treatments | Exemplary Readouts |
|---|---|
| Injury | Viability (metabolic activity assay) |
| (puncture wound) | Morphology (histology - H&E, immunostaining) |
| | Chemokines/cytokines expression |
| | Omics data (transcriptomic, proteomics, |
| | metabolomics) |
| Irritation/Corrosion | |
| Phototoxicity/ | |
| Photosensitivity | |

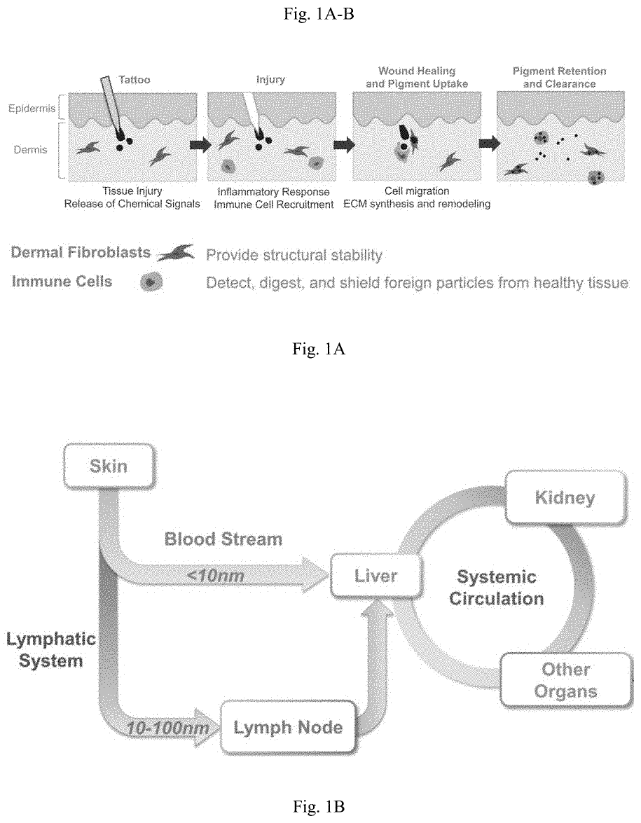

FIG. 1A-B shows an illustrative schematics for embodiments of wound healing, e.g. by tattoo needles, tattoo gun, etc.

FIG. 1A illustrates epidermal and dermal injury, followed by epidermal and dermal healing. Tattoo pigments are retained within the dermal matrix and dermal fibroblasts. Pigments may also be well as transported downstream, with or without immune cells through the lymphatic and/or circulatory system thereby causing systemic effects.

FIG. 1B illustrates how agents distribute in the body from the skin to downstream lymphatic and/or circulatory system fluidic connections thereby potentially causing systemic effects, e.g. effects upon lymph node cell activation (or suppression), effects upon liver, kidney, lung brain, etc. Numbers refer to exemplary sizes of cells/particles. Exemplary effects upon skin include but are not limited to: cytotoxicity; phototoxicity; sensitization/allergen; corrosivity/irritancy; carcinogenicity, etc. Exemplary systemic effects leading to effects upon other organs include but are not limited to: cytotoxicity; carcinogenicity; effect on metabolism/organ function, etc. In some embodiments, downstream microfludic devices simulating organs, such as lymph node-chips, liver-chips, kidney-chips, intestine chips, etc. are used for testing effluent from treated Skin-chips. In some embodiments, organ chips are fluidically connected downstream of a treated Skin-Chip. In some embodiments, organ chips are tested directly, i.e. individually, as one example for testing systemic effects of pigments.

A. Skin-Chip: Recreating Native Human Skin In Vitro.

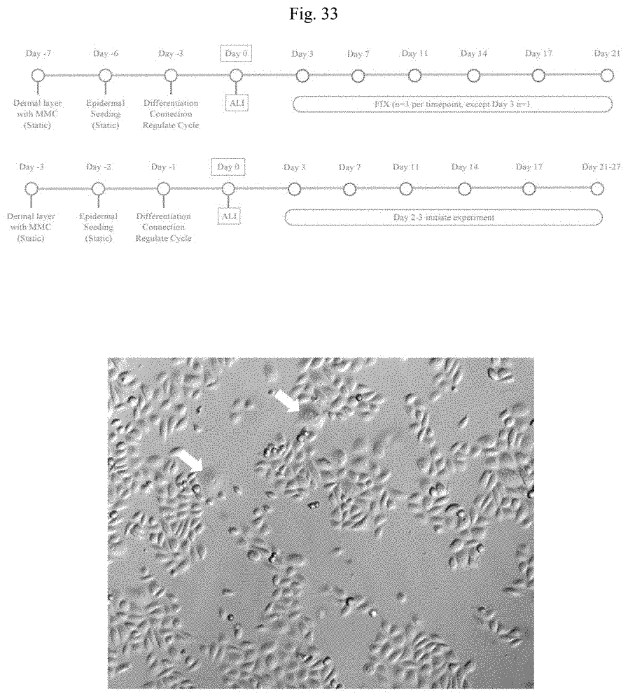

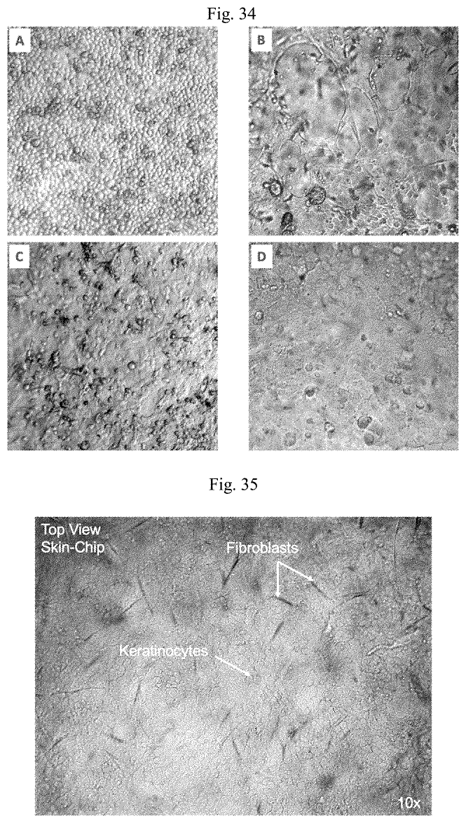

In a microfludic skin-chip, parenchymal cells include skin epithelial cells and dermal cells, while stromal areas may include fibroblasts, melanocytes, cells of the sweat glands, cells of the hair root, and any combinations thereof, for creating an in vitro skin mimic. One embodiment of a procedure for the preparation, seeding, and maintenance of a Skin-Chip Model is described herein. See, FIG. 33.

In some embodiments, this protocol applies to the preparation of Open-Top stretchable Skin-Chips, by seeding 2 cell types: dermal fibroblasts and epidermal keratinocytes. In some embodiments, Skin-Chips comprise additional cells such as fibroblasts, melanocytes, cells of the sweat glands, cells of the hair root, and any combinations thereof. In some embodiments, Skin-Chips comprise additional cells such as white blood cells, e.g. immune cells. White blood cells include but are not limited to resident white blood cells isolated from skin biopsies, circulating immune cells isolated from blood, white blood cells as cell lines including but not limited to macrophage-like cells derived from cancer patients.

Keratinization or cornification of the upper epithelial layer occurs through media composition (introduction of growth factors and Ca2+) as well as switching the skin-Chip from a liquid-liquid flow system to air-liquid flow system. In some embodiments, cornification is induced by culturing under an air-liquid interface without continuous flow.

In some embodiments, a microfluidic device used for a Skin-chip is an open-top chip with a stretchable PDMS membrane, e.g. an open-top stretchable chip. In some embodiments, an open-top stretchable Skin-chip as described herein, has a spiral shaped vascular channel. In some embodiments, an open-top stretchable Skin-chip as described herein, has a spiral shaped vascular channel with known types of structural anchors for providing gel support.

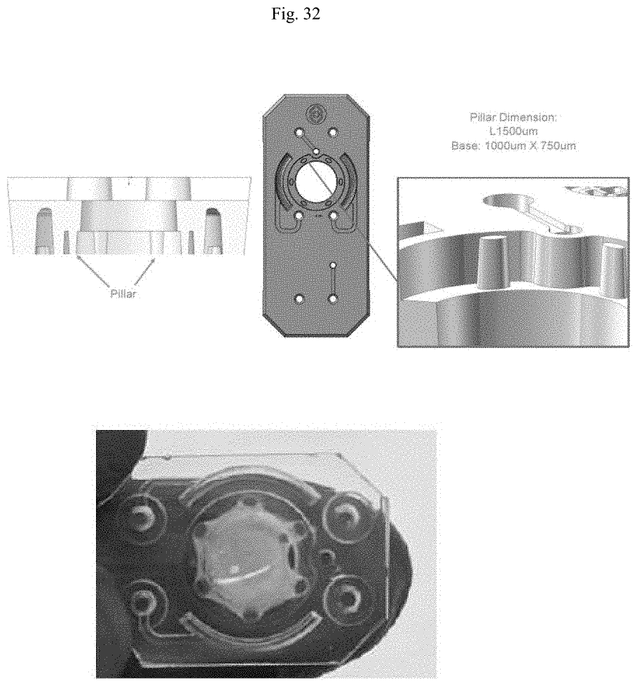

In some embodiments, an open-top stretchable Skin-chip as described herein, has design improvements contemplated for overcoming problems with gel shrinkage when culturing under an ALI. In one embodiment, an improvement is on anchor designs, e.g. six large anchors as pillars, and/or a serpentine shaped vascular channel. Thus, in one emboidment, an improved open top (OT) microfluidic device has a 6-(large) pillar chamber. In one emboidment, an improved open top (OT) microfluidic device has a 400 um×400 um bottom channel. In one emboidment, an improved open top (OT) microfluidic device has both a 6-pillar chamber, for gel anchoring, and a a 400 um×400 um bottom channel with a serpentine shape instead of a spiral shape. An exemplary large pillar is 750 um×1000 um at the base, then tapers up; and 1.5 mm tall. See, FIG. 32.

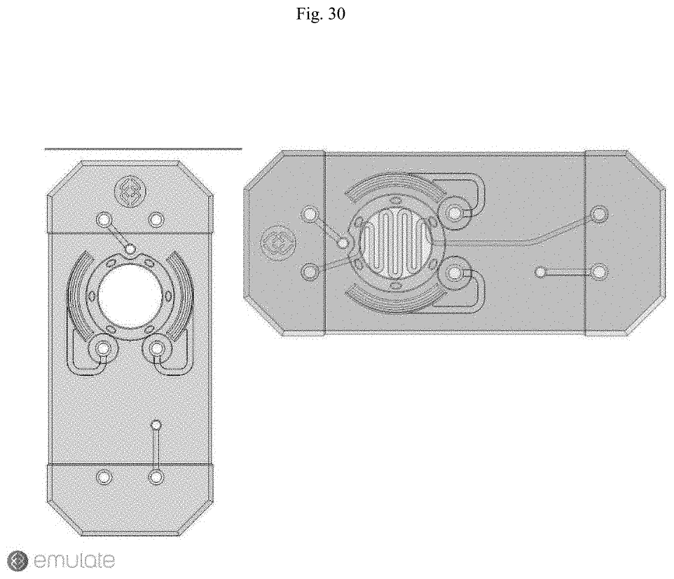

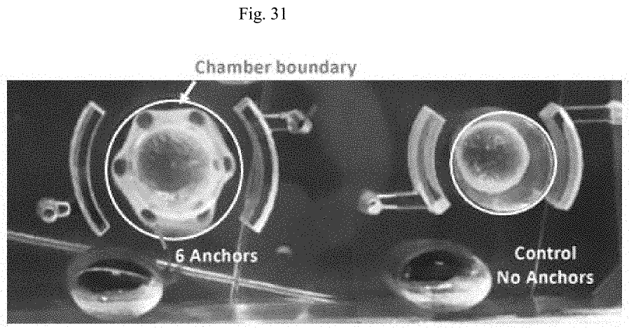

Thus, design changes for an improved OT chip may apply to both basal and apical compartments, e.g. addition of large gel anchoring pillars within the open top chamber, altering the shape of the bottom chanel from spiral configuration to a speretine configuration. See, FIGS. 30, 31 and 32. Contemplated improvement results over previous microfludic open top chip designs include but are not limited to better gel stability (mitigates contraction and delamination issues) and minimizes bubble formation within bottom channel. See, FIG. 32. One exemplary timeline contemplated for use with an improved microfluidic OT chip. See, FIG. 33.

In some embodiments, a device used for a skin-chip is an open-top chip with a (stretchable) PDMS membrane, also referred to as a Hybrid (H) Skin-Chip:Transwell.

In some embodiments, this protocol applies to the preparation of Hybrid (H) Skin-Chips:Transwells, referred to herein as “TW” or “HTW” or “HSK-TW” or “transwells” or “Skin-Chips”. Such that in one embodiment, a HSK-TW refers to a device having a stretchable membrane and chamber for a dermal fibroblast gel overlaid with keratinocytes, as described herein.

In one embodiment, a conventional Transwell culture system, which does not have a stretchable membrane or fluid flow, comprises a 6.5 mm transwell with 0.4 μM Pore Polyester Membrane Insert, Sterile (Corning, Ca #: 3470).

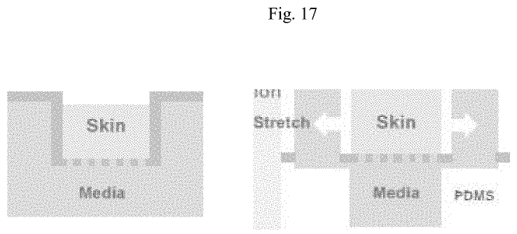

TABLE 1B
In Vitro Skin Models: Test Platforms. See, FIG. 17.

| System | Description | Purpose |
|---|---|---|
| Conventional | Transwell device | Conventional platform for |
| | without a capability | culturing skin, e.g. an |
| | for membrane | epidermal layer formed by |
| | stretching | keratinocytes and dermal cells, |
| | (nonstretchable | pieces of skin biopsies, etc. |
| | membrne) nor | |
| | continuous fluid | |
| | flow. | |
| Hybrid (H) | (Static) PDMS Chip | Platform for culturing skin, |
| Transwell | with a stretchable | e.g. forming and maintaining a |
| (HTW) | membrane without | full-thickness epidermal layer |
| | the use of stretch or | formed by keratinocytes and |
| | continuous fluid | dermal cells. |
| | flow. | Provides a test platform |
| | | with same device |
| | | material properties as a |
| | | microfluidic Open-Top |
| | | chip. |
| Open-Top | Dynamic PDMS | Provides dynamic culture |
| Chip | Chip with a | condition using |
| | stretchable | stretch and under continuous |
| | membrane but | fluid flow. |
| | without the use of | |
| | stretch and with the | |
| | use of continuous | |
| | fluid flow in both | |
| | channels until ALI- | |
| | then the upper | |
| | channel does not | |
| | have continuous | |
| | flow. | |

In some embodiments, microfluidic Skin-Chips and HSK-TW devices are used for understanding tattoo permanence and pigment clearance: Following epidermal healing, tattoo pigments are retained within the dermal matrix and dermal fibroblasts, as well as transported downstream via immune cells and the circulatory system.

Wound healing cascade and pigment retention in Skin-Chip assessed using Tattoo pigments and Fluorescent beads, e.g. Tattoo on Skin-Chip.

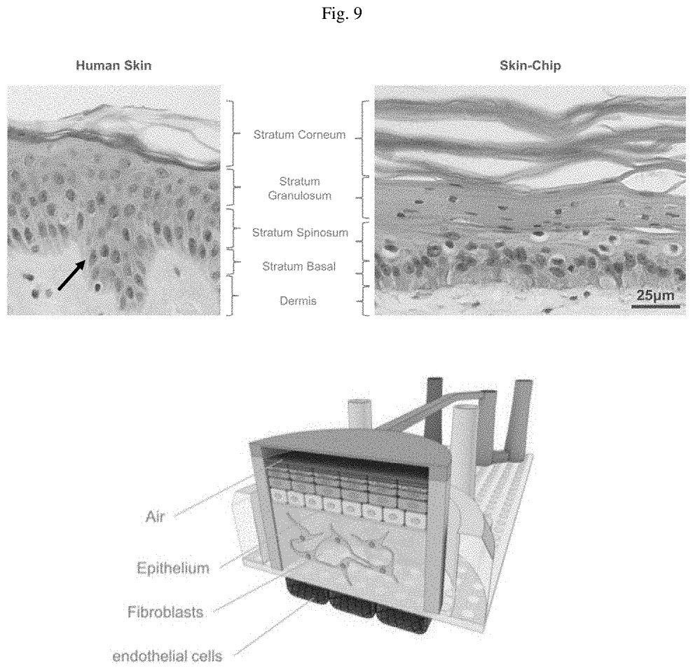

FIG. 9 shows exemplary comparative images of skin in a biopsy (left panel) and in a Skin-chip (right panel), created without using Ficoll™, stained using H&E. An exemplary schematic of one embodiment of an open-top Skin Chip is illustrated below the microscopic images.

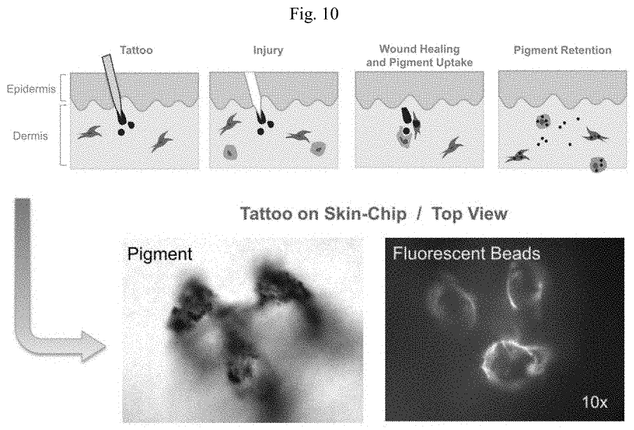

FIG. 10 illustrates an exemplary schematic of a microfluidic chip comprising skin, wounded by a tattoo needle, followed by healing, pigment uptake, clearance. wound healing cascade and pigment retention in Skin-Chip assessed using Tattoo pigments and fluorescent beads. Tattoo on Skin-Chip/Top View.

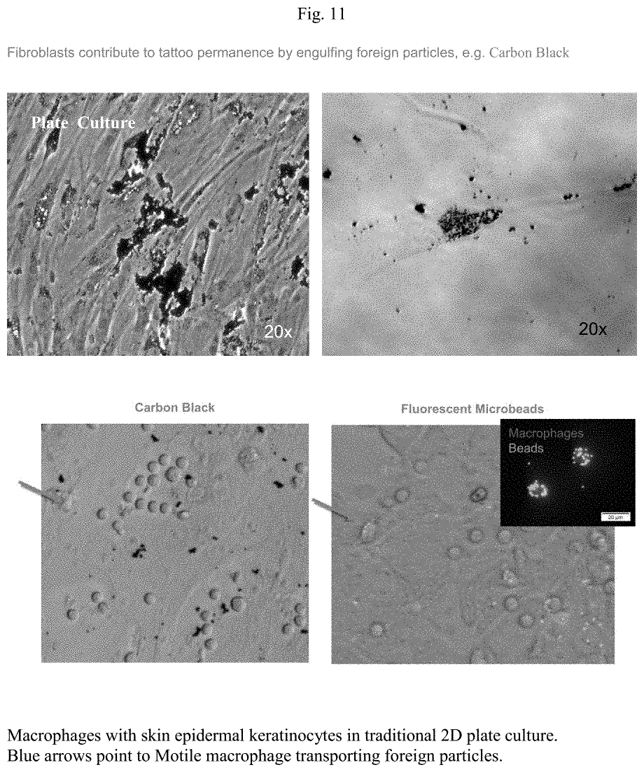

FIG. 11 shows exemplary dermal cells (fibroblasts) uptake ink and fluorescent beads in plate culture (upper panels) and in a Skin-chip, lower panels. Fibroblasts contribute to tattoo permanence by engulfing foreign particles. Fibroblast actively uptake and retain tattoo pigments and fluorescent particles of all size ranges.

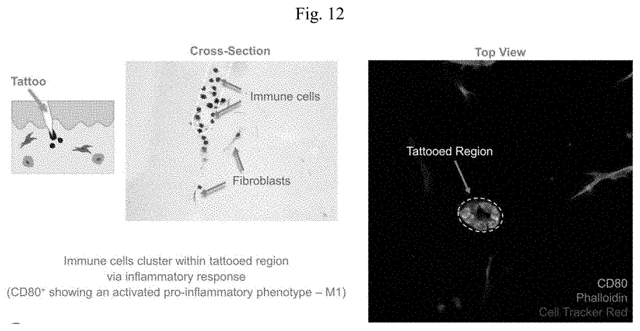

FIG. 12 shows exemplary immune cell activation within wounded region as an in vitro immune response: Phagocytosis of Foreign Particles. Schematic, left panel, illustrating ink particles in a dermal region (light pink) through an epidermal tattoo wound (clear) in the epidermis (dark pink). Microscopic image middle showing part of a dermal region in a Skin-Chip where blue arrows point to CD80+ dark stained immune cells, e.g. a MV4-11 macrophage cell line (e.g. (ATCC® CRL-9591™) added to the dermal region, that cluster within tattooed region via inflammatory response (CD80+showing an activated pro-inflammatory phenotype—M1). MV4-11 macrophage cell line was derived from a human having a biphenotypic B myelomonocytic leukemia disease where cells appear to be lymphoblasts in culture. Blue arrows below the wound point to elongated fibroblasts. A top view of the skin chip, i.e. looking down, showing stained cells in and in relation to a wounded tattoo region. CD80 (blue), Phalloidin (pink) and Cell Tracker (red) stained cells, red. Immunofluorescently stained CD80+ cells are clustered within the wounded tattoo region in the right panel.

The Skin-Chip demonstrates for the first time wound healing capabilities in an in vitro human skin model, recapitulating physiological response to tattoo injury through a cascade of immune response, cell activation and migration, and ECM remodeling.

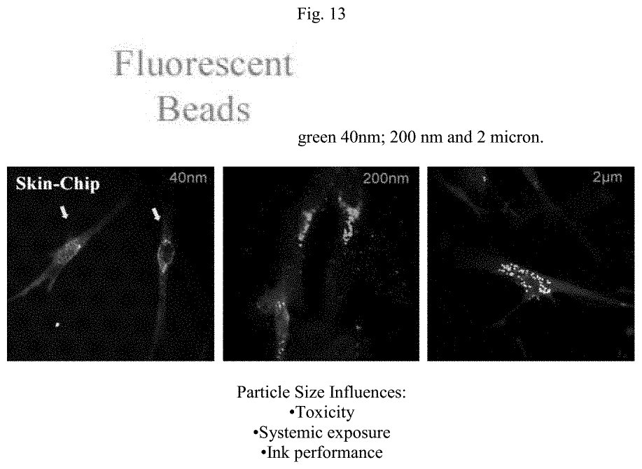

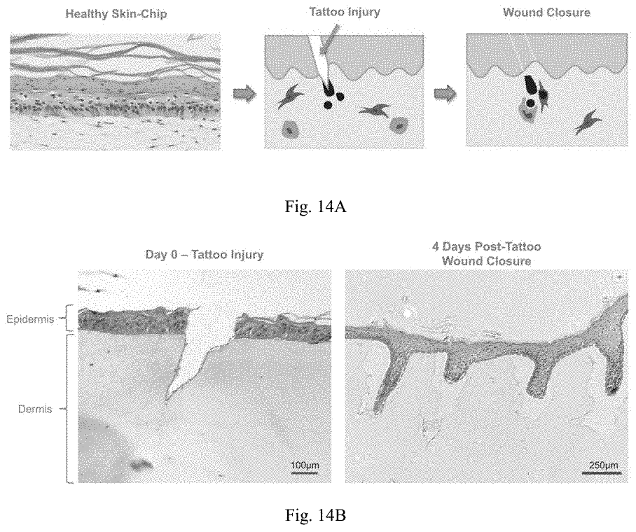

FIG. 13 shows exemplary immune cell (macrophage) activation within a tattooed area of a Skin-Chip. A range of fluorescent particles are taken up by cells. Size range vs. color?

FIG. 14A shows an exemplary full-thickness healthy Skin-Chip used for recreating human in vivo skin function, e.g. wound healing.

FIG. 14B shows exemplary microscopic images of H&E stained cross sections of one embodiment of a Skin-Chip showing wound healing following puncture wounds. A full-thickness Skin-Chip is able to heal through epidermal and dermal remodeling of wound site. Day 0—tattoo injury. 4 Days post-tattoo wound closure.

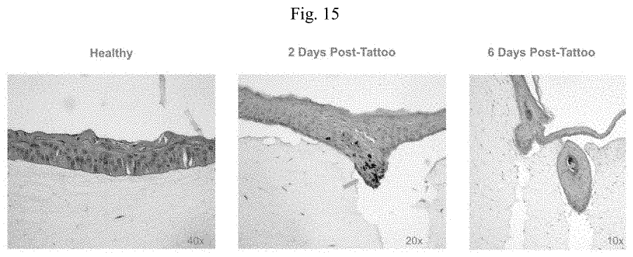

FIG. 15 shows exemplary Tattoo on Skin-Chip: pigment shedding through the epidermis. Healthy 2 days post-tattoo 6 days post-tattoo epidermal and dermal remodeling during wound healing show trapped pigment within epidermis, which become isolated and pushed upwards as the epidermal cells go through their cycle of stratification towards to upper most layer of the skin (normal skin cycle=14 days).

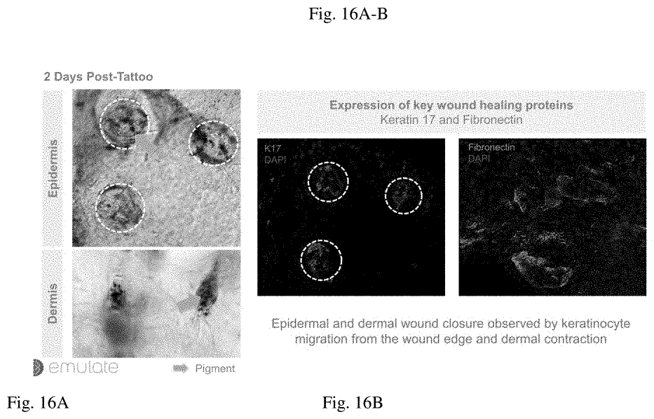

FIG. 16A-B-shows exemplary epidermis and dermis remodeling during wound healing.

FIG. 16A shows exemplary micrographs of epidermis and dermis 2 Days Post-Tattoo. Blue arrow points to pigment.

FIG. 16B shows exemplary immunofluorescent micrographs demonstrating expression of key wound healing proteins Keratin 17 (pink) and Fibronectin (green). Nuclei colored blue. Epidermal and dermal wound closure observed by keratinocyte migration from the wound edge and dermal contraction.

FIG. 17 shows exemplary schematic illustrations of a conventional transwell device, left, having skin (dermis and epidermis)-orange, media-pink, separated by a nonstretchable porous membrane-blue dotted line, fluid was changed using a pipette; and a hybrid transwell chip device for mimicking conditions in a microfluidic chip, however with the absences of stretching and fluid flow, with a surface area smaller than a conventional transwell, right. Skin (dermis and epidermis)-orange, media-pink, separated by a stretchable porous membrane-blue dotted line. Fluid was changed using a pipette. In some embodiments, a microfluidic chip was used as a HTW, however again there was no stretch or continuous fluid flow, microchannels may be briefly flushed using microfluidic systems to provide fresh media.

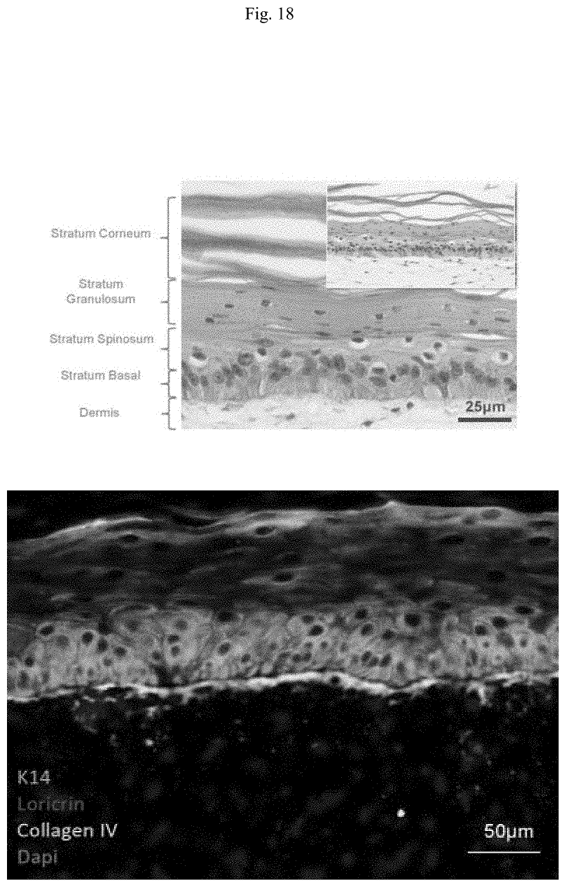

FIG. 18 shows an exemplary microscopic image demonstrating full-thickness skin that forms within a static chip platform, inset shows a higher magnification. Immunofluorescent image demonstrates exemplary biomarkers for keratinocyte layers, e.g. K14 (green) and loricrin (red) and ECM, i.e. Collagen IV.

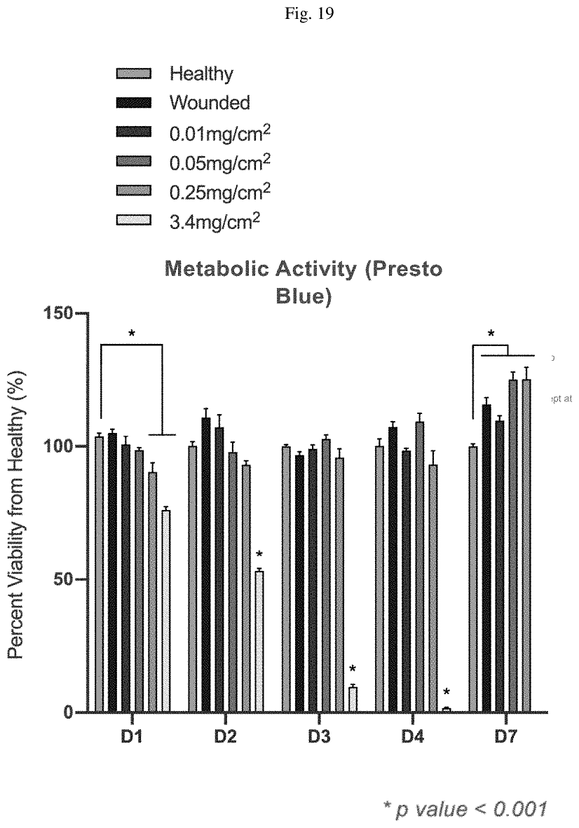

FIG. 19 shows exemplary Toxicity in the Skin-Chip: TiO₂ Tattooed on Static Skin-Chip Model. Metabolic activity (Presto Blue). * p value<0.001. Tattooed on D4 at ALI, N=4 per condition Hybrid Transwells.

Dose-dependent response 24 h post-tattoo observed by a decrease in viability with increasing TiO₂ concentration. Skin recovery observed over 7 days, except at highest dose leading to tissue necrosis.

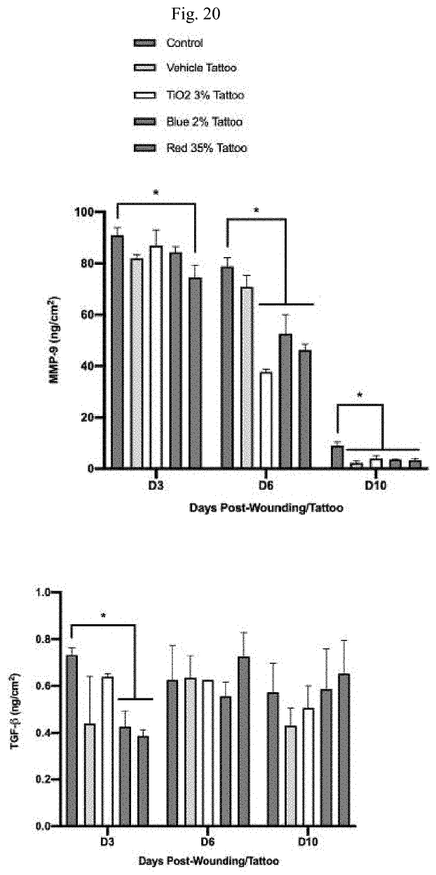

FIG. 20 shows exemplary wound repair biomarkers MMP-9 and TGF-B after tattooing with TiO2, Blue and Red pigments. Hybrid Transwells.

MMP-9 was expressed after wounding during keratinocyte migration towards the wound edge, and by dermal fibroblasts through ECM degradation and invasion. Presence of tattoo pigments impedes production of MMP-9 involved in wound repair, slowing down the repair process. TGF-B inflammatory cytokine controls procollagen expression. Need to assess TGF-B expression at later time point post-tattoo (D14, D17). Tattooed on D4 at ALI N=3 per condition Hybrid Transwells.

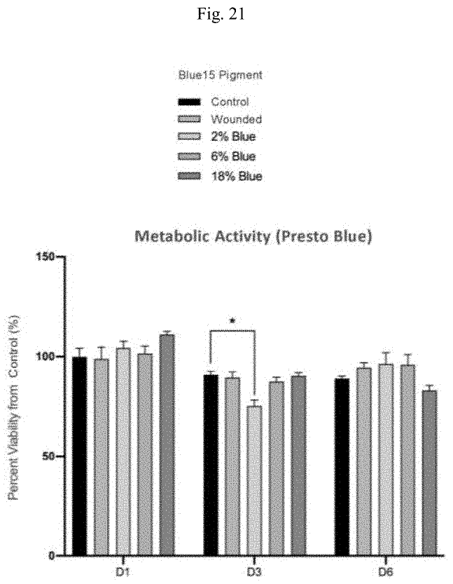

FIG. 21 shows exemplary Toxicity of Blue15 Tattooed on Static Skin Model. Metabolic Activity (Presto Blue) of Blue15 Pigment. Tattooed on D4 at ALI. N=4 per condition Hybrid Transwells. Control; Wounded; Percent viability from Control (%). Blue15 pigment does not cause observable changes in tissue viability over 6 days post-tattoo.

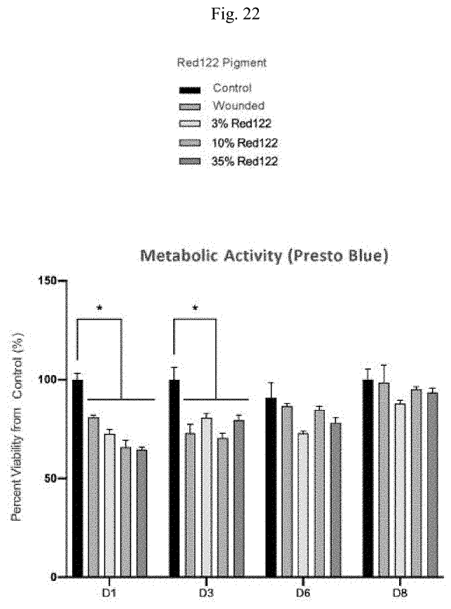

FIG. 22 shows exemplary Metabolic Activity (Presto Blue) Red122 Pigment. Concentration-dependent response 24 h post-tattoo observed by a decrease in viability with increasing Red122 concentration. Skin recovery observed over 8 days post-tattoo Tattooed on D4 at ALI N=4 per condition Hybrid Transwells.

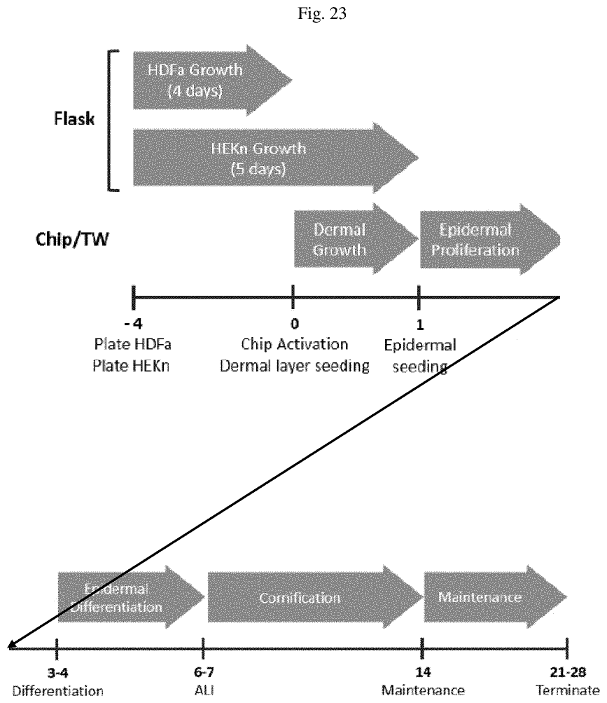

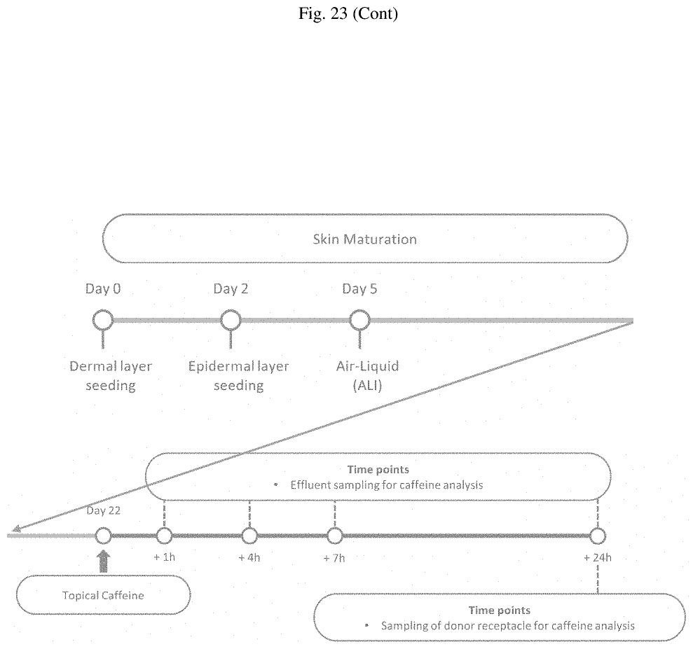

FIG. 23 shows exemplary schematic timelines for providing full-thickness epidermal layers in Hybrid Transwells, along with an exemplary read out points. Lower timeline shows exemplary compound testing, e.g. permeability compounds Testosterone and Caffeine.

As used herein, “full-thickness” in reference to an artificial skin, e.g. as provided in a full-thickness Skin-Chip, refers to a stratified epidermis including a bassement membrane, e.g. identifiable by detection of extracellular matrix proteins including but not limited to collagen IV, Laminin 5, etc., stratum basal layer, Keratin 14, Stratum spinosum: Keratin 10, Stratum granulosum: Filaggrin, Stratum corneum: Involucrin, Loricrin

Tissue robustness evaluated by determining the ET50 value following topical exposure to 1% TritonX-100. Static Skin model resulted in ET50 value around 11 h, compared to skin equivalent models on the market (e.g. MatTek—7 h).

### Tissue Quality and Robustness: Exposure to Irritant (TritonX-100).

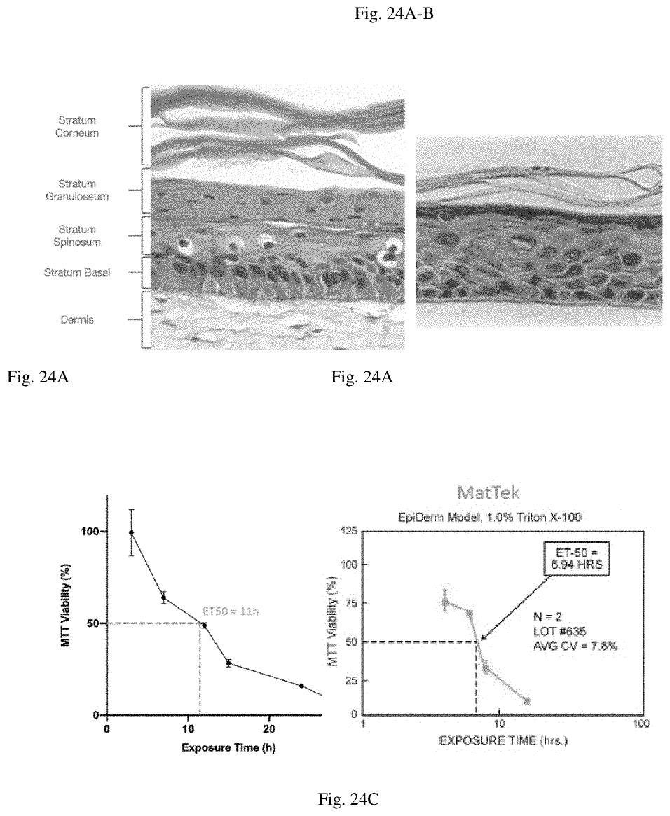

FIG. 24 A-C shows exemplary micrographs comparing FIG. 23A Skin chip (static) D18 in culture (D14 at ALI), FIG. 23A EpiDerm200 from MatTek. FIG. 23A shows that ET50 for one embodiment of a Skin-Chip is better at ET50=11 hours vs. around 7 hours with EpiDerm200 from MatTek.

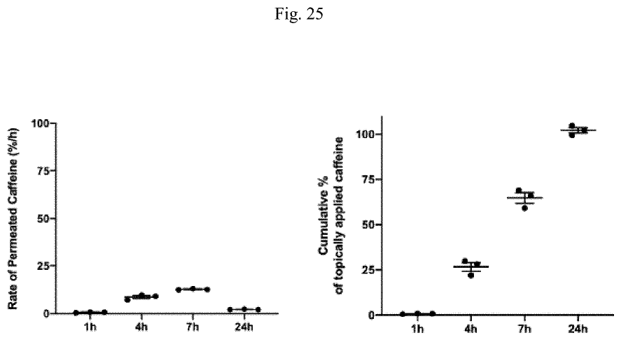

FIG. 25 shows an exemplary Caffeine Permeability. D14 ALI N=3 per condition Hybrid Transwells.

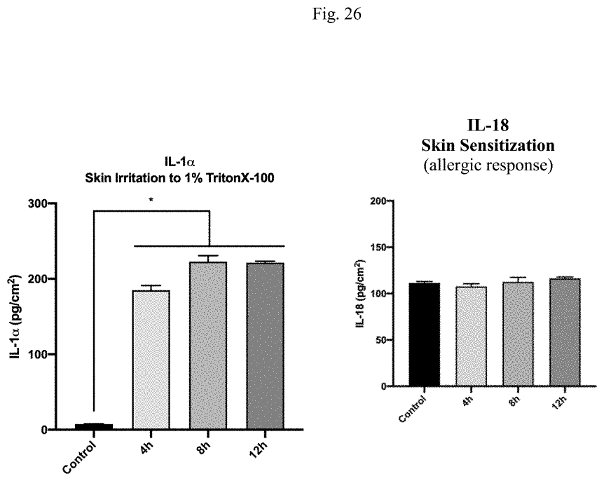

FIG. 26 shows an exemplary Skin Irritation—Release of Associated Cytokines. Topical exposure to a known skin irritant, Triton X-100, shows increase in expression of inflammatory cytokine IL-1alpha. IL-18, expressed following skin sensitization, shows no significant increase after exposure to Triton X-100.

D11 ALI. N=3 per condition Hybrid Transwells.

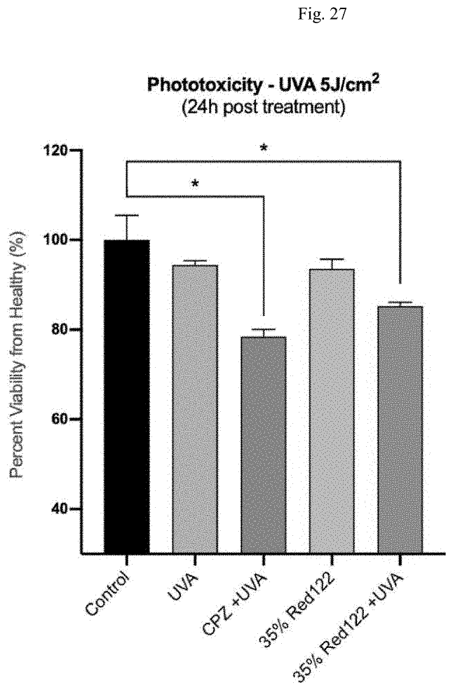

FIG. 27 shows an exemplary Safety Assessment of Red Tattoo Pigment—Phototoxicity Assay (Viability). Red tattoo pigments known to cause some level of toxicity following sun exposure. Static Skin models were exposed to minimal dose of UVA which does not cause tissue damage (5 J/cm²). Samples tattooed with 35% w/v of Red122 pigment showed some phototoxicity with a decrease in viability to 85% from healthy. D12 ALI (D8 post-tattoo) N=4 per condition. Hybrid Transwells.

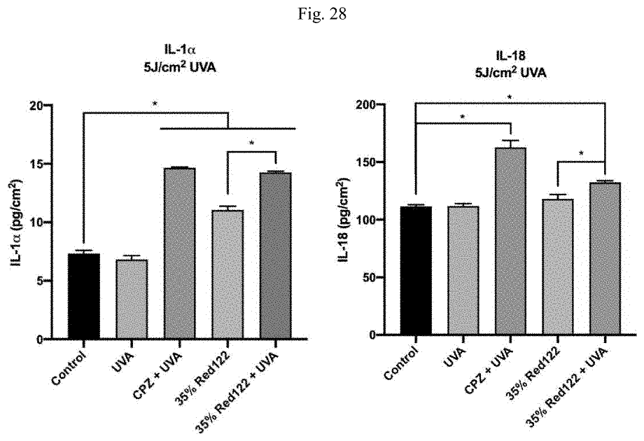

FIG. 28 shows an exemplary Skin tattooed with 35% w/v of Red pigment showed an increased secretion of inflammatory cytokine, but no effect as a skin sensitizer. Following UVA exposure, tattooed samples showed increase in both cytokines, indicating that Red is both phototoxic and a skin sensitizer under UVA. UVA on D11 ALI (D7 post-tattoo) N=3 per condition. Hybrid Transwells.

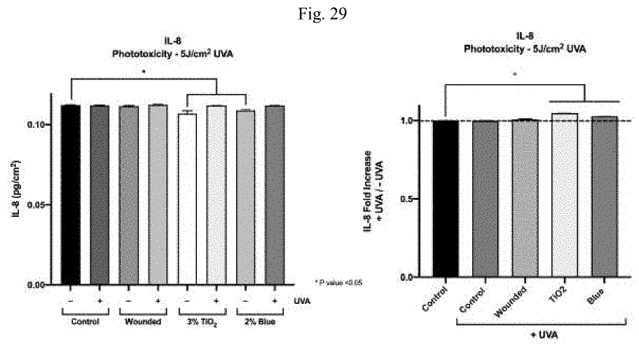

FIG. 29 shows an exemplary Phototoxicity to TiO₂ and Blue15—Cytokines Release. * P value<0.0001. UVA on D18 ALI (D14 post-tattoo). N=3 per condition. Hybrid Transwells.

FIG. 30 shows one embodiment of an examplary improved open top device having a 6-pillar chamber. Pillars may be from 4 to 12, however in part due to mechanical difficulties during pipetting gels and subsequent problems of gel contraction, a 6-pillar design provided optimal conditions. Pillar Dimensions: 750 um×1000 um at the base (tapers up); 1.5 mm tall Chamber height 3 mm with a 400 um×400 um Bottom channel. shows an exemplary schematic illustration of methods for providing a full-thickness Skin-Chip comprising new design features, compatible with a biological incubator and Zoe.

FIG. 31 shows one embodiment of an examplary improved open top device having Open-Top Chip design changes to both basal and apical compartments.
Addition of gel anchoring pillars within the open-top
Modification of bottom channel from spiral configuration

Resulting from these improvements:
Gel stability (mitigate contraction and delamination)
Minimize bubble formation within bottom channel

FIG. 32 shows one embodiment of an improved open top device. Lower panel shows a photographic image of an improved Skin-Chip comprising new design features resting on a gloved finger (purple).

FIG. 33 shows exemplary embodiments of shorter timelines for use with microfluidic Skin-chips, e.g. using a cell culture device (e.g., Zoe). showing an exemplary bright filed image of keratinocytes sultures prior to seeding in Skin-Chips. An optimal kertinocyte cell population should be non-differentiated keratinocytes forming packed islands, with minimal number of differentiated keratinocytes characterized by a rounded morphology and double in size

| Experimental Parameters | Protocol |
|---|---|
| Regulate Cycle | Performed once during initial L/L connection |
| Introducing ALI | 200 uL/hr for 5 min |
| Flow Rate | 60 uL/hr |

FIG. 34A-D shows exemplary microscopic imges of cells after seeding. FIG. 34A) Keratinocyte seeding (D0), FIG. 34B) Slight epidermal contraction leading to holes, significantly more noticeable when cultured on stiff gels (bovine collagen type I 5 mg/mL) (D1), FIG. 34C) Differentiation medium (D3), FIG. 34D) Air-liquid interface ALI (D4).

FIG. 35 shows exemplary microscopic representative image of a confluent epidermal monolayer on top of a dermal layer (D3 post-keratinocyte seeding).

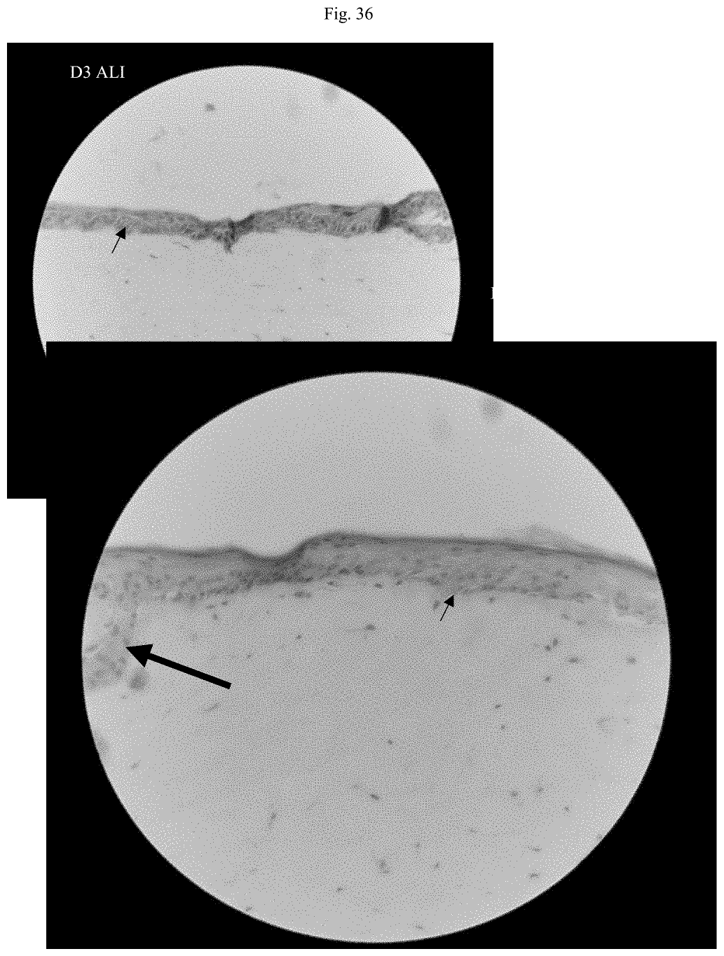

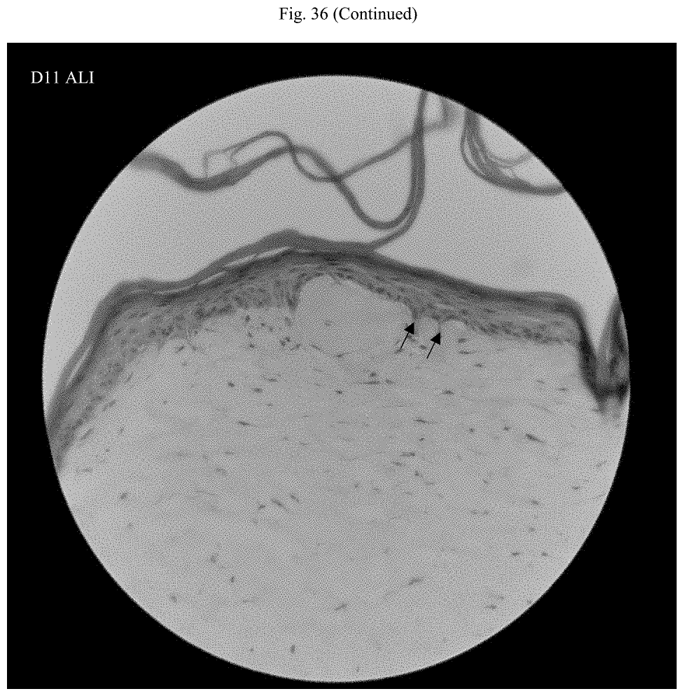

FIG. 36 shows exemplary microscopic images of H&E stained cross sections of fixed, e.g. formalin fixed, epidermal and dermal regions formed after D3 ALI, D7 ALI and D11 ALI in microfluidic Skin-Chips, wherein Ficoll™ was added to the dermal gel, as described herein. Arrows point to surprising epidermal-dermal invaginations, where the epidermis appears to be embedded (integrated) within the dermal region. Thse types of invaginations were not observed in cross-sections of skin from devices without Ficoll™, see for one example, FIG. 9.

Incorporation of improved design features reduced gel contraction, however gel matrix contraction around pillars (leading to slight flooding) occurred from around day 14 after beginning ALL.

Surprisingly there was better KC attachment to the dermal region, and appeared to have more convolutions (e.g. invaginations) when compared to not using Ficoll. As used herein, “epidermis” refers to an epithelial tissue layer of skin, comprising in vivo, hair follicles, sebaceous glands, sweat glands, etc, which are types of numerous epithelial invaginations of epidermis into dermal regions, see, for example Human skin FIG. 9.

Additional observations: ALI was maintained over 21 days, up to at least 27 days; however after around Day 14 ALI a flush cycle was needed to clear flooding every 3-4 days due to some gel contraction around pillars (leading to slight flooding). Bubble occurrences in the bottom channel was minimal, e.g. 2/12 chips once every 7 days.

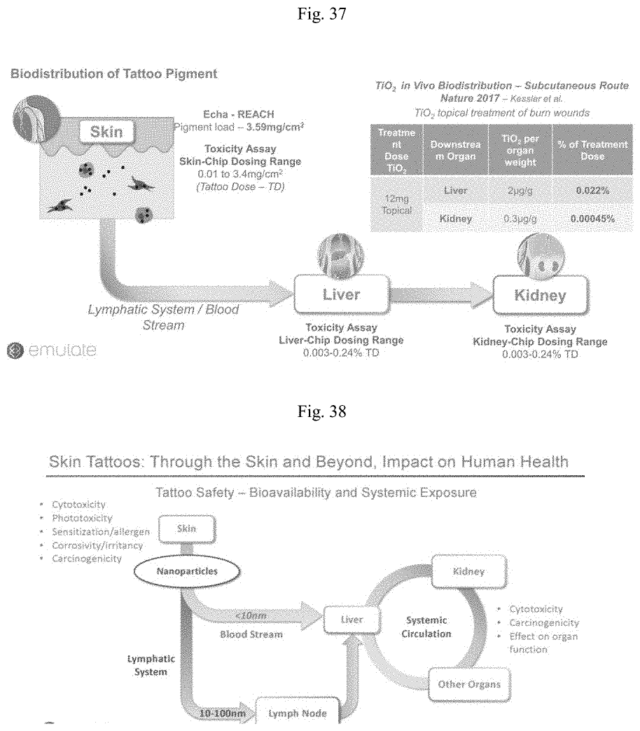

FIG. 37 shows exemplary schematic embodiments for testing biodistribution of tattoo pigments or other particulate test compounds between the skin—liver and kidney. FIG. 18 shows exemplary Assessing Tattoo Pigment Safety using Organ-Chips: Biodistribution of Tattoo Pigment. TiO₂ topical treatment of burn wounds.

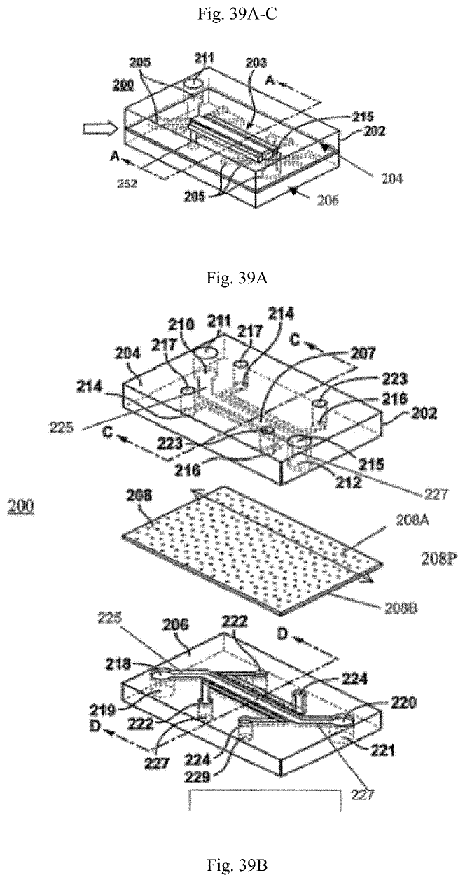

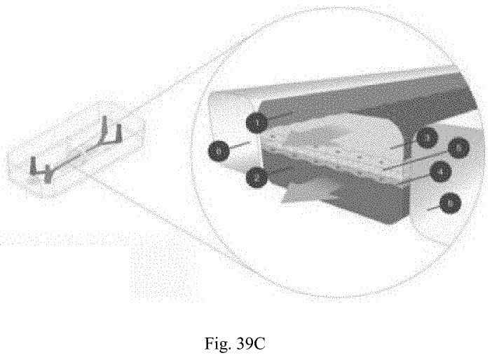

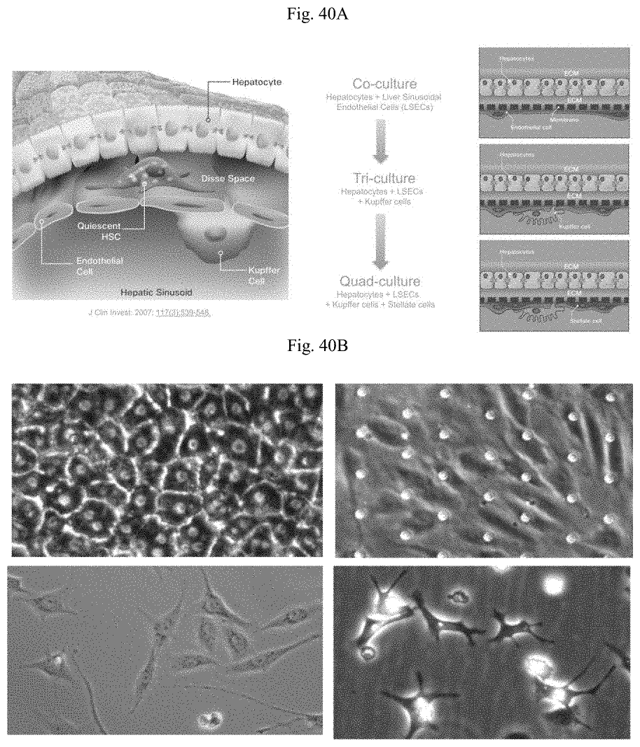

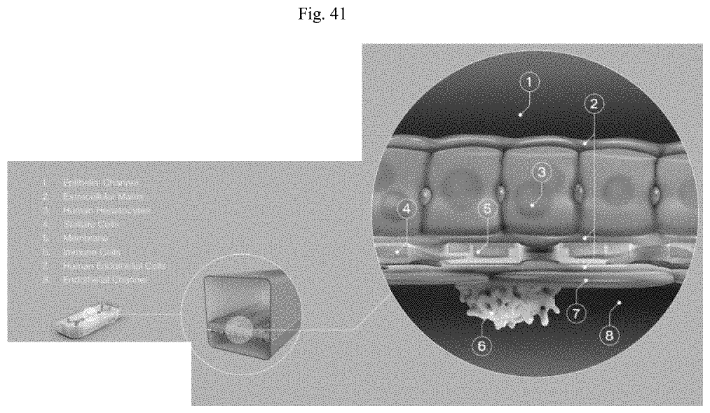

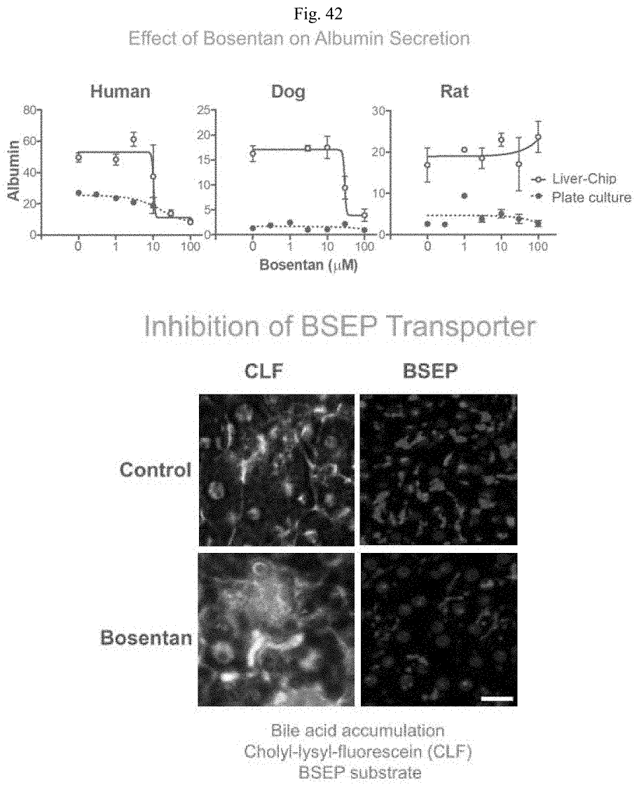

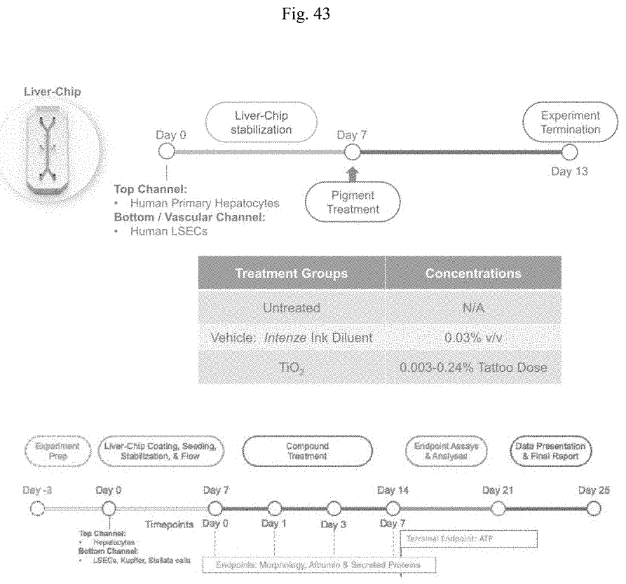

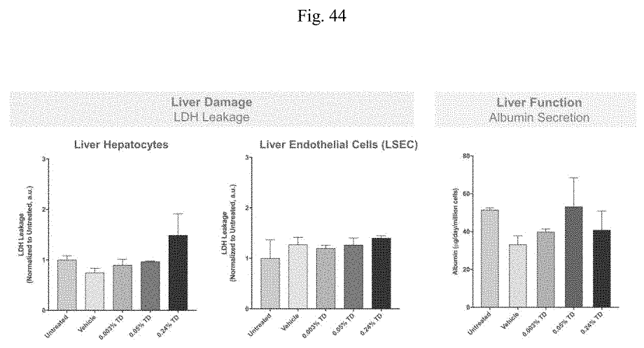

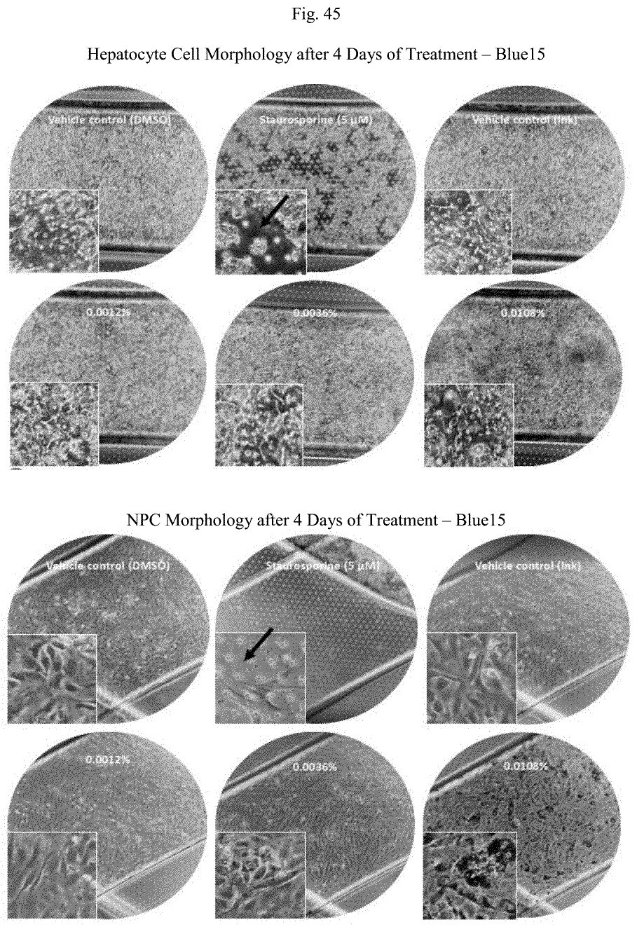

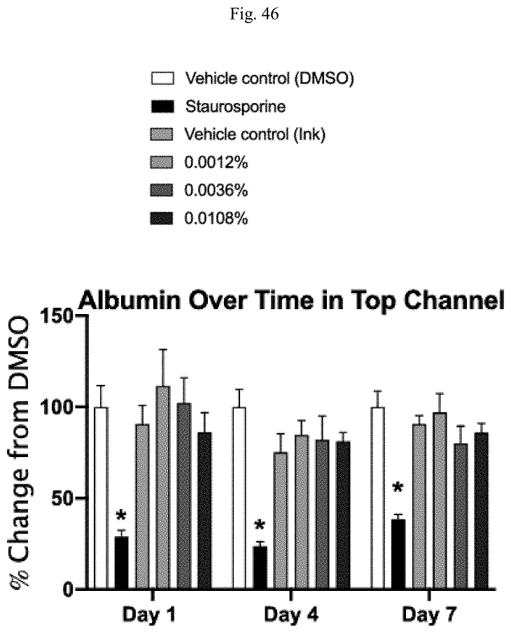

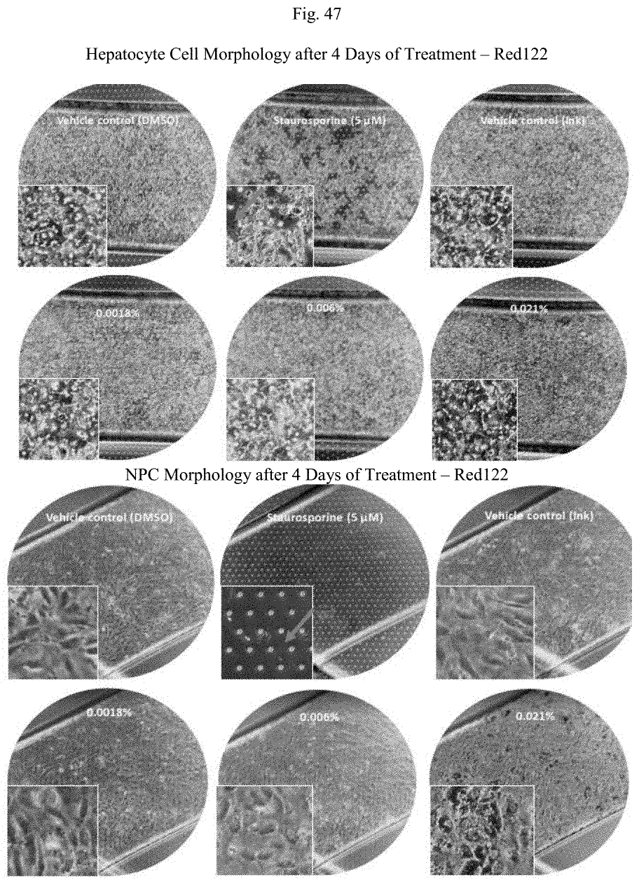

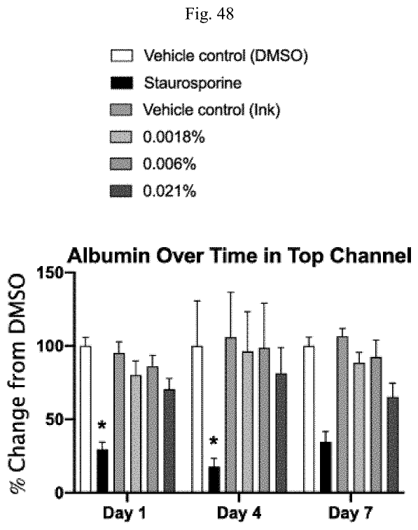

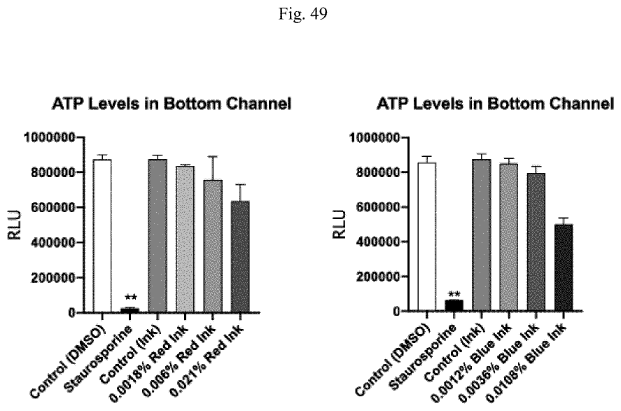

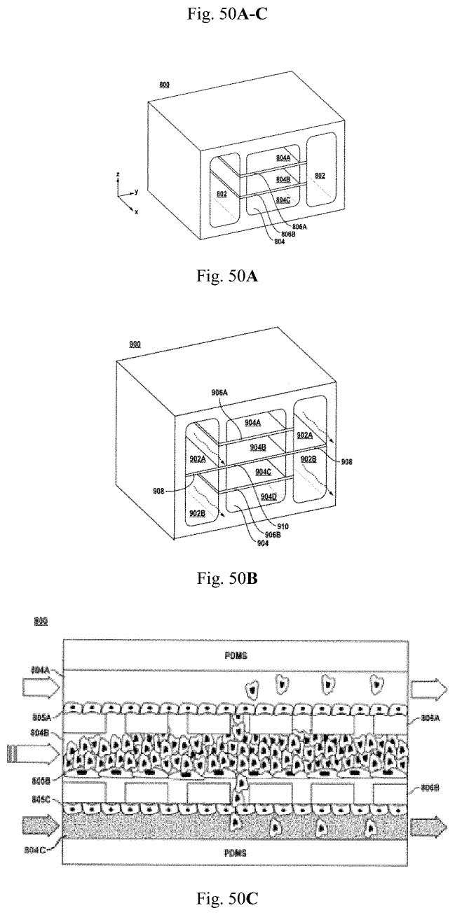

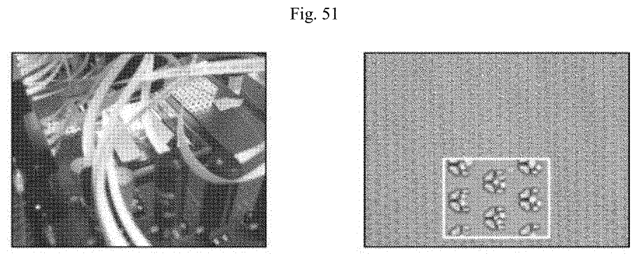

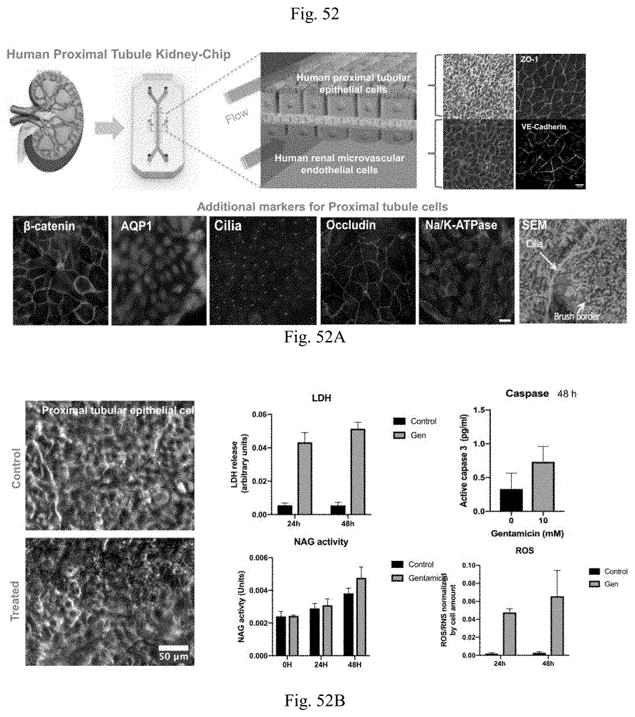

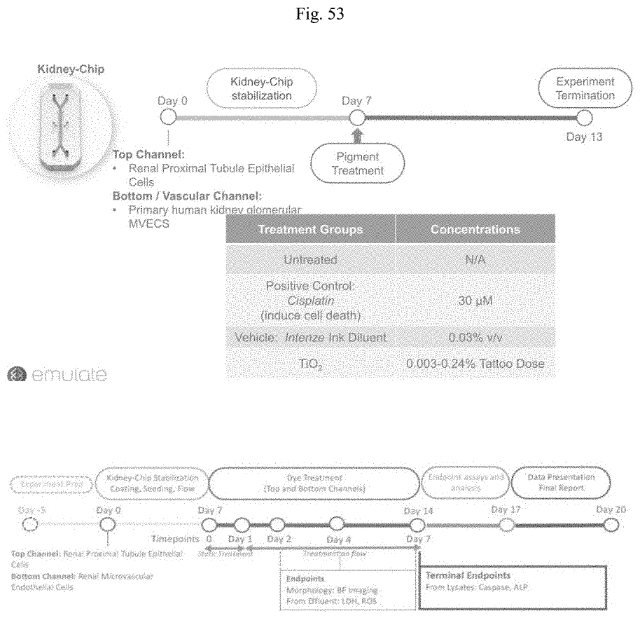

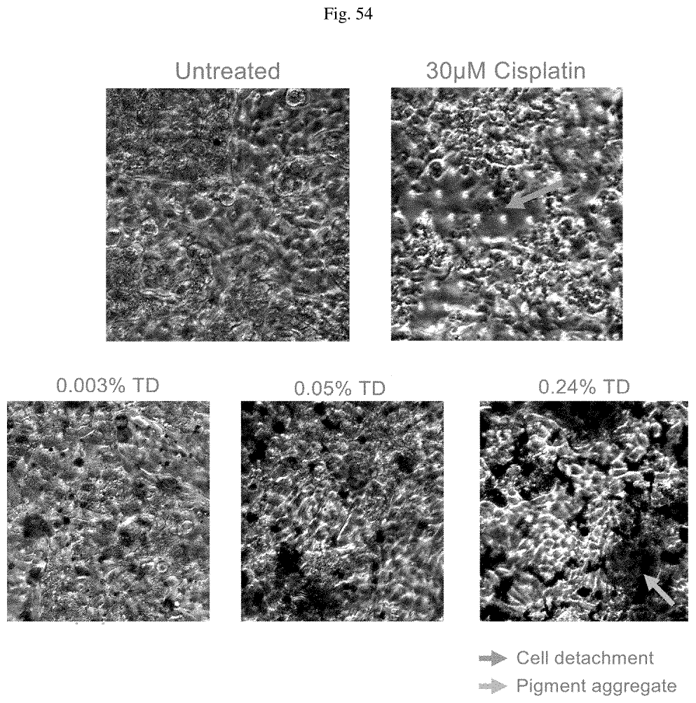

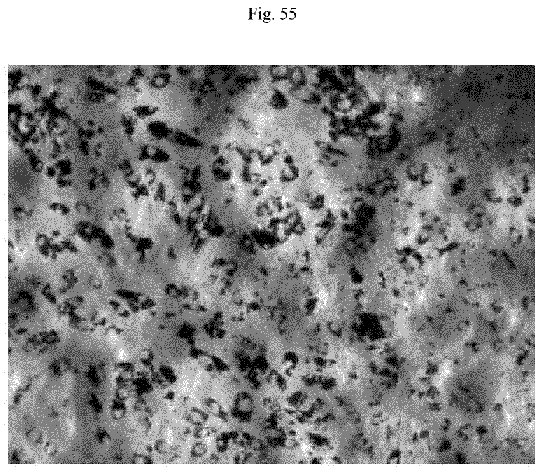

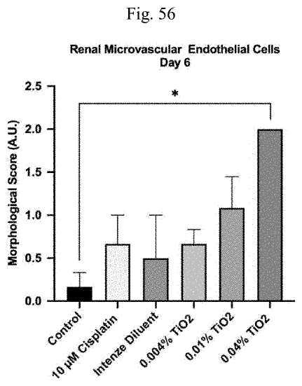

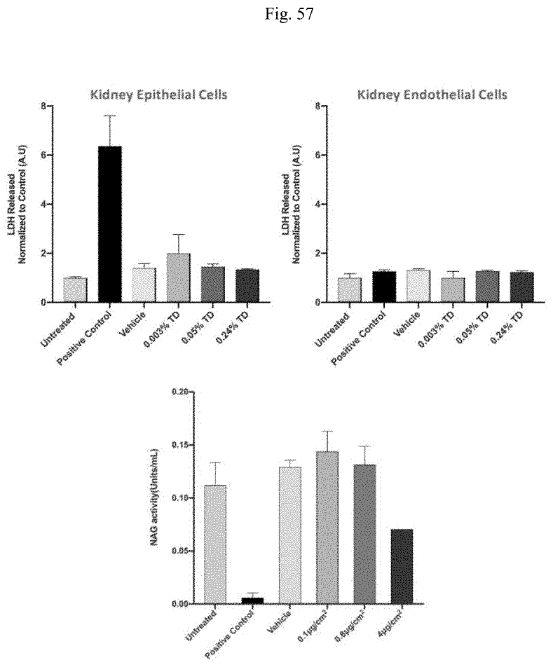

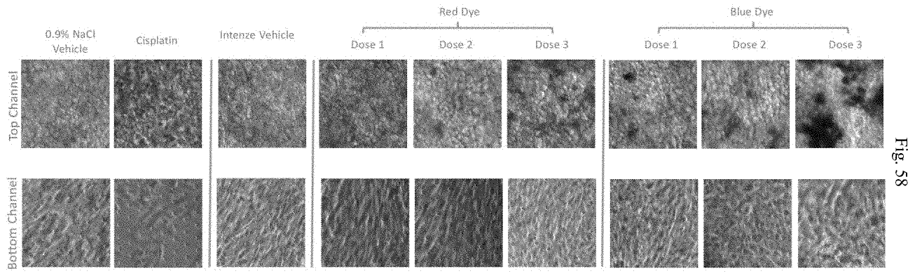

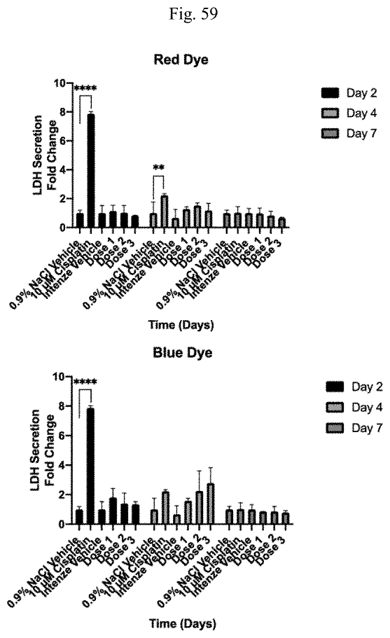

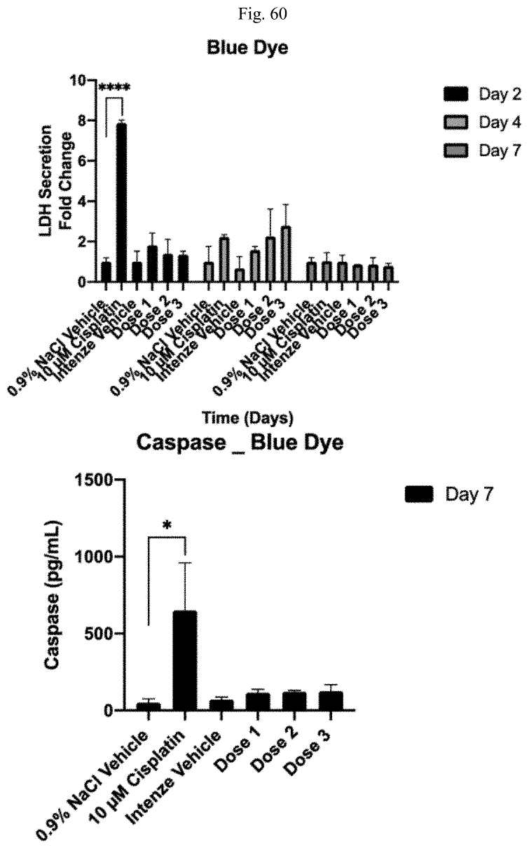

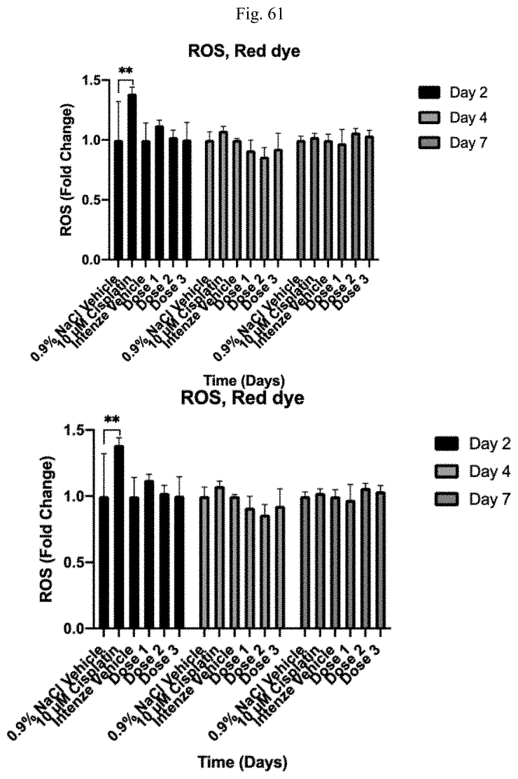

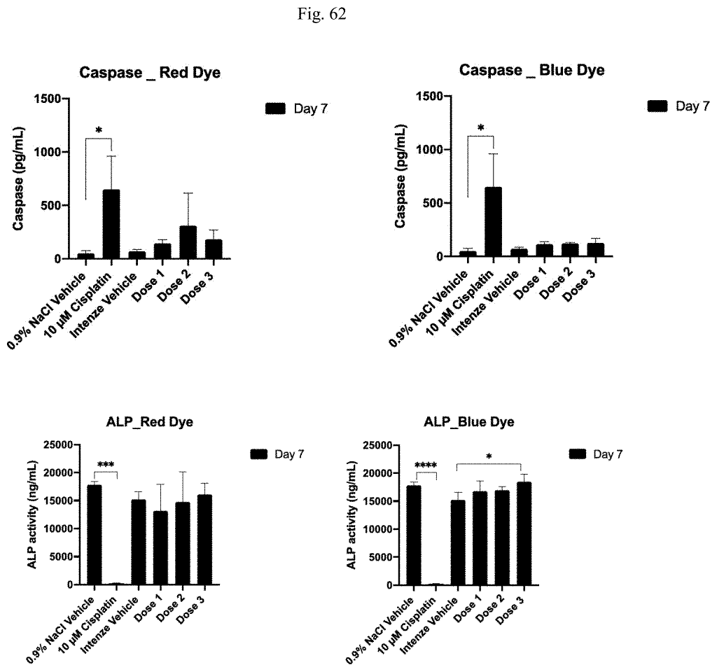

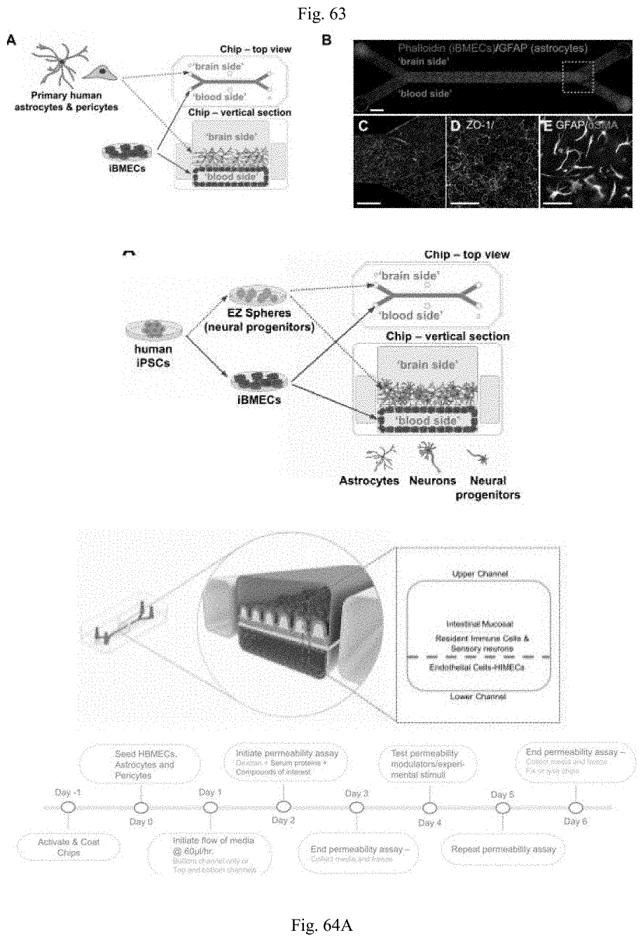

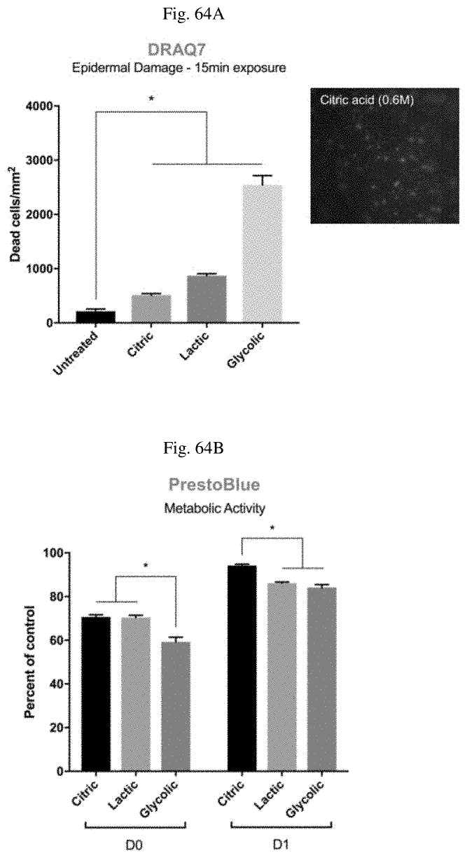

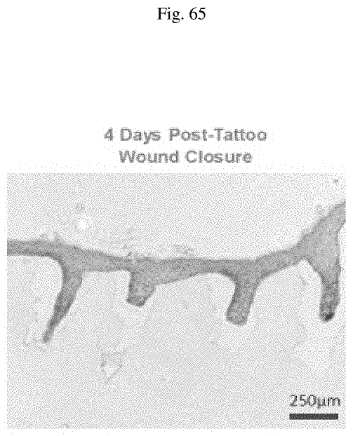

FIG. 38 shows exemplary schematic embodiments for testing biodistribution of tattoo pigments or other particulate test compounds between the skin—lymph node—liver—kidney and other organs, such as brain and intestine.

### Example 1—Chip Activation and Coating (See Additional Method Embodiments Herein)

Day 0: Chip Activation and Coating (this is a highly time-sensitive step). Collagen solutions must be prepared fresh and stored on ice until use.
Chip activation: Prepare 1 mg/mL ER1 in ER2, protect from light; Rinse chip with ER2 (do not aspirate immediately, leave ER2 in chip for 5-10 min); Fill chip with ER1 and expose to UV light for 10 min.; Aspirate ER1, fill chip with fresh ER1 and expose to UV light for 10 min; then Rinse 3 times with ER2 and aspirate chip dry

### Example 2—Dermal (Fibroblast) Cell Culture

basement membrane (BM) papillary and reticular dermis separated by a vascular plexus, the rete subpapill are; dermal papilla region of the follicle and along its shaft.

A. Dermal Fibroblast Cells.

Dermal cell (dermal) proliferation medium—ThermoFisher provides per request: +1% Pen/Strep; +5% FBS; +50 μg/mL Sodium Ascorbate (Sigma, Ca #A4034-100G).

Dermal gel preparation: Harvest fibroblasts with Tryple-E for 6 min at 37 C; Centrifuge for 5 min at 200 g. Perform cell count and resuspend fibroblasts in dermal proliferation medium at 2×106 cells/mL.

Fibroblasts growth medium—DMEM high glucose with Glutamax (ThermoFisher, Ca #10569)+10% FBS. Refresh medium every 2 days.

Fibroblasts—expansion in 1×T25 (250,000 cells). Cell number sufficient for n=18 TW or chips.

Fibroblasts may be trypsinized using 0.05% trypsin/EDTA (Corning 25-052 CL) according to protocol described above. One can then re-suspend the fibroblast pellet in the predetermined amount of 10×DMEM or variants. This is mixed with the necessary amount of reconstitution buffer. (Note: best results are obtained when fibroblasts are collected in active growth phase, which occurs when fibroblast are between 50 and 70% confluence).

100 μl ECM+fibroblast are added to each well and this is incubated (37° C. for 2 Hours). Thereafter, 100 μl of E medium is added to the top of each collagen gel. 100 μl of E medium+RM TG* is then added to the bottom of each collagen gel. This is incubated (37° C. for 12-16 Hours).

A variety of collagen containing matrices are contemplated for use in making an artificial derma and ECM to embed fibroblasts: Tropoelastin: Collagen I: Collagen III: Dermatan sulfate (1 mg:3 mg:3 mg:0.5 mg); Col I (3 mg/ml)/Elastin (3 mg/ml); Col I (3 mg/ml)/Elastin (1 mg/ml); Col I (10 mg/ml)/MaxGEL; Col I (3 mg/ml)/Elastin (3 mg/ml) 1:1 MaxGel; Col I (3 mg/ml)/Elastin (3 mg/ml)/Col III (3 mg/ml) 1:1:1; MaxGel; Col I (10 mg/ml)/Elastin (10 mg/ml); etc.

B. Preparing the Dermal Layer.

Dermal gel casting: 90 ul gel solution per chip, 180 ul gel solution per TW. 500 ul of bovine collagen I . . . ; 275 ul DMEM (5% FBS) containing a mixture of Ficoll MW70 and Ficoll MW 400 (37.5 mg/mL and 25 mg/mL respectively); 100 ul HDFa cells (human dermal fibroblasts) in suspension in fibroblast growth media (at 2×10\hat{ }6 cells/mL). Final collagen concentration of 5 mg/mL. All reagents should be kept on ice. Incubate overnight in Dermal growth medium. Additionally: 10×MEM to 10× Recon Buffer to Collagen volumes should be kept as 1:1:8.

Example volumes for mixing a collagen stock solution of 10 mg/mL, final gel volume of 1 mL, as one example: 62.5 ul 10×MEM (Sigma, Ca #M4655); 62.5 ul 10× Recon Buffer (made in-house—Antonio); 2.2 g sodium bicarbonate in 75 ml of 0.067M NaOH; Add 4.76 g HEPES; Can be stored for up to 1 year at −20° C.; 500 ul of bovine collagen I (Advanced BioMatrix, FibriCol® Type I Collagen, Cat #5133-20ML) at 10 mg/ml; 275 ul DMEM (5% FBS) containing mixture of Ficoll 70 and Ficoll 400 (37.5 mg/mL and 25 mg/mL respectively); 100 ul HDFa suspension (at 2×106 cells/mL).

In general, when beginning, pipette tips are cooled by putting into refrigerator for 15-30 min (Pipettes need to be cold when working with rat-tail type I collagen in order to avoid coagulation). Both the pipette tips and the ECM matrix should stay in an icebox or other cooler during the procedure.

In order to calculate the final volume of rat-tail type I collagen mixture needed, one calculates the number of dermal equivalent cultures that are needed. This calculation is based on 12 well+3 extra (those are needed to compensate for the ECM matrix that adheres to the surface of pipette). Where 2×10⁴ neonatal or adult Human Foreskin Fibroblast per raft are employed and 12+3 rafts are prepared, one needs 15×2×10⁴=30×10⁴ fibroblasts (or 300,000 fibroblasts). To impede fibroblasts proliferation, one can irradiate the fibroblast with 70Gy.

To make 150 μl/raft×(12+3) rafts=2.25 ml. 10% 10×DMEM or variants*=0.225 ml or 225 μl. 10% reconstruction buffer⁺=0.225 ml or 225 μl. 80% ECM matrix=1.8 ml or 1800 μl. (1.8 ml ECM matrix×2.4×10 1N NaOH (1M))=43.2 μl 1M NaOH (1M) (NaOH makes ECM matrix to coagulate). This is put into incubator 37° C. for 2-4 Hours.

In one embodiment, place the liquid gel containing fibroblast cells and other desired cell types intended for the dermal/stromal area, directly into a device for gelling in place. In one embodiment, place the liquid gel containing fibroblast cells and other desired cell types intended for the dermal/stromal area into a mold for providing a preformed fibroblast-gel plug to place into a device.

C. Keratinocyte Cells (Keratinocytes).

Epidermal proliferation medium—ThermoFisher (DMEM/F12 3:1).

### +1% Pen/Strep

+0.3% chelated FBS (chelation using Chelex 100 Sodium form and 200-400 dry mesh size, Biorad 142-1253)
+50 μg/mL Sodium Ascorbate (Sigma, Ca #A4034-100G)
+0.628 ng/mL Progesterone (Sigma, Ca #P7556-100MG
+10 ng/mL hrEGF (ThermoFisher, Ca #PHG0311L).
Epidermal differentiation medium—ThermoFisher (same as epidermal proliferation medium, DMEM/F12 3:1)

### +1% Pen/Strep

### +0.3% FBS

+50 μg/mL Sodium Ascorbate (Sigma, Ca #A4034-100G)
+0.628 ng/mL Progesterone (Sigma, Ca #P7556-100MG
+115 μg/mL CaCl2) (Sigma, Ca #C5670)
Cornification medium—ThermoFisher (DMEM/F12 1×)+1% Pen/Strep; +2% FBS; +50 μg/mL; and Sodium Ascorbate (Sigma, Ca #A4034-100G).
Maintenance medium—Thermo Fisher (DMEM/F12 1×)+1% Pen/Strep+1% FBS.
Keratinocytes—expansion in 1×T75 (500,000 cells per flask). Cell number sufficient for n=18 TW or chips.
Keratinocyte This example describes the preparation of keratinocytes, and in particular human foreskin keratinocytes (HFKs). In some embodiments, human primary epidermal keratinocytes, are commercially obtained, e.g. neonatal from foreskin (ATCC, Ca #PCS-200-010). For example, keratinocyte cells passaged up to P4 (passage 4) after initiation of primary keratinocyte cultures, are used for seeding Skin chips.

Keratinocyte growth medium—Dermal cell basal medium (ATCC, Ca #PCS-200-030)+Keratinocyte growth kit (ATCC #PCS-200-040).

In some embodiments, Day 1: Epidermal Seeding comprises replacing medium on chip or transwells with epidermal growth medium. Harvest keratinocytes with Tryple-E, incubate at 37 C for 8-10 min (gently tap plate to help detach the cells); centrifuge for 5 min at 150 g. Aspirate media from gel surface and let dry in incubator. Perform cell count and resuspend keratinocytes in epidermal proliferation medium at 5×106 cells/mL. Seed keratinocytes on chips and TW (30 ul of cell suspension on TW, 25 ul of cell suspension on chip). Incubate for 2 h to allow for cell attachment. Rinse twice with epidermal growth media to remove unattached keratinocytes. Refresh media and leave overnight.

### Exemplary Timelines for

Day 0: seed dermal fibroblast cells in a dermal gel. See, herein. In one embodiment, the liquid gel containing fibroblast cells, and other desired cell types intended for the dermal/stromal area, is flowed directly into a device for solidifying in place as a dermal gel. In one embodiment, the liquid gel containing fibroblast cells, and other desired cell types intended for the dermal/stromal area, is flowed into a mold for providing a preformed solidified dermal gel as a gel plug to place into a device.
Day 1: seed epidermal (keratinocyte) cells by placing the keratinocytes on top of the dermal gel. The protocol shows a microscopic image of a confluent epidermal monolayer on top of a dermal layer.

### Day 3-4: HEKn Differentiation

Aspirate and replace media with epidermal differentiation medium Note: Switch to differentiation medium can be done earlier depending on epidermal monolayer confluence—monitor daily and assess quality based on reference images B and C. Day 6-7: ALI

Aspirate dry gel surface and replace basal medium with cornification medium, exposition the smkin to Air-Liquid Interface (ALI)

Note: Switch to ALI can be done 1 or 2 days after differentiation medium; assess based on quality of epidermal monolayer. Day 10-11: Day 4 at ALI.

Physical injury (wound healing) assay can be performed at this stage, over a period of 7 days.
Day 14: Maintenance media; Switch to maintenance medium. Topical treatment and other challenges can be performed at this stage, over a period of 7 days.

An aliquot of Lonza Gold KGM media (Lonza 192060) is placed in a 50 ml tube (i.e. with 1 cryovial of HFK cells, one needs 12 ml for the flask, 10 ml for the washing step and 1 to 5 ml to break the pellet for a total of about 25 ml).

The medium is warmed by putting it into the water bath for 5-10 min. and then transferred inside the sterile hood. The 15 and 50 ml conical tubes are prepared as needed, along with flasks. These are filled with the appropriate amount of Lonza medium.

To thaw the HFKs, a cryovial is removed from the liquid nitrogen container and transferred into the basket containing dry ice. The cryovial is placed into the water bath until the freezing medium inside it is completely melted. The cryovial is sprayed with ethanol and brought to the sterile hood. The cryovial is opened in the hood and the contents are collected from the cryovial (freezing medium+ cells) using a 1000 μl pipette. The contents are transferred into the 15 ml conical tube containing Lonza Gold KGM medium previously warmed. This conical tube is closed and then tilted to mix. Thereafter, it is centrifuged at 1000 rpm for 5 minutes. The conical tube is sprayed with ethanol and returned to the sterile hood. It is opened and the supernatant is withdrawn, leaving the cell pellet. The pellet is re-suspended using fresh pre-warmed Lonza Gold KGM and the mixture is transferred to a flask (or flasks), which were previously filled with Lonza Gold KGM medium. The flasks are gently agitated to make sure that the medium covers the entire bottom surface. The flasks are then transferred to the incubator. The keratinocytes are fed with new media approximately every other day (about every 36 hours).

To thaw the fibroblasts, a cryovial is removed from the liquid nitrogen tank and transferred into the basket containing dry ice. The cryovial is placed into the water bath until the freezing medium inside it is completely melted. The cryovial is sprayed with ethanol and brought to the sterile hood. The cryovial is opened in the hood and the contents are collected from the cryovial (freezing medium+ cells) using a 1000 μl pipette. Tee contents are transferred into the 15 ml conical tube containing Lonza FGM-2 medium previously warmed. This conical tube is closed and then tilted to mix. Thereafter, it is centrifuged at 1200 rpm for 5 minutes. The conical tube is sprayed with ethanol and returned to the sterile hood. It is opened and the supernatant is withdrawn, leaving the cell pellet. The pellet is re-suspended using fresh pre-warmed Lonza FGM-2 and the mixture is transferred to a flask (or flasks) that were previously filled with Lonza FGM-2 medium. The flasks are gently agitated to make sure that the medium covers the entire bottom surface. The flasks are then transferred to the incubator. The fibroblasts are fed with new media approximately every other day (about every 36 hours).

For detaching the HFKs by trypsinization, the protocol is as follows. First, an aliquot Lonza Gold KGM (Lonza 192060), Lonza reagent subculture reagent CC-5034 and E-medium (or variants) 10% FBS medium is placed in 15 ml and 50 ml tubes. It is convenient to us 4 mls of Lonza reagent subculture reagent CC-5034 per T75 flask and to add 8 mls of 10% FBS medium to the flask (which corresponds to 2 ml for each ml of reagent Lonza reagent subculture reagent CC-5034). The media and enzymes are warmed by putting it into the water bath for 5-10 min. The flask containing HFK (typically when the cells are between 50 and 70% confluence) is removed from the incubator, sterilized on the outside with ethanol, and transferred into the hood. The flask is opened and the the Lonza Gold KGM medium is aspirated, being careful to not scratch the bottom flask surface where the cells are attached. Fresh pre-warmed Lonza Gold KGM medium (e.g. 5 mls) is then added to wash the cells. This media is also aspirated carefully. Then, 4 ml of 0.05% trypsin/EDTA (Corning 25-052 CL) is added to the flask and the flask is returned to the incubator. The detaching cells can be monitored using the microscope if desired. As a rule of thumb, keratinocytes should detach in about 2-3 minutes. Longer exposure to Lonza subculture reagent CC-5034 (or 0.05 EDTA trypsin Invitrogen 25200-056) could damage keratinocytes irreversibly. When the cells detach completely, the outside of the flask is sterilized and brought to the hood. The flask is opened and 8 ml of 10% FBS E-medium (or variants) is added to the flask (2 ml for each ml of 0.05 EDTA trypsin Corning 25-052-CL). Thereafter, the contents of the flask are conveniently transferred to a 15 ml conical tube. The tube is closed and centrifuged at 1000 rpm for 5 min. The tube is then sterilized with ethanol, returned to the hood and opened. The supernatant is gently aspirated, being careful not to disturb the cell pellet. After the supernatant is removed, the pellet is re-suspended using fresh pre-warmed Lonza Gold KGM medium. The mixture is then transferred to the flask/flasks, which were previously filled with Lonza Gold KGM medium. The flasks are gently agitated to make sure that the medium covers the entire bottom surface, and they are returned to the incubator. Feeding is as stated above.

For detaching the fibroblasts by trypsinization, the protocol is as follows. An aliquot of Lonza FGM-2 medium (Lonza CC-3132), Lonza reagent subculture reagent CC-5034 and 10% FBS medium is added in 15 ml and 50 ml tubes. It is convenient to use 4 ml Lonza reagent subculture reagent CC-5034 per T75 flask and 8 ml of 10% FBS medium to the flask (which corresponds 2 ml for each ml of reagent Lonza reagent subculture reagent CC-5034). The media and enzymes are warmed by putting them into the water bath for 5-10 min. The flask containing fibroblasts (typically when the cells are between 50 and 70% confluence) is removed from the incubator, sterilized on the outside with ethanol, and transferred into the hood. The flask is opened and the media is aspirated gently, being careful to not scratch the bottom flask surface containing the cell layer. 5 ml of fresh PBS is added to wash the cells (this can be done twice). The PBS is aspirated carefully, and 4 ml of 0.05% trypsin/EDTA (Lonza CC-5012) is added and the flask is returned to the incubator. The detaching cells can be monitored using the microscope if desired. As a rule of thumb, fibroblasts should detach in about 2-3 minutes. Longer exposure could damage the cells irreversibly. When the cells detach completely, the outside of the flask is sterilized and brought to the hood. The flask is opened and 8 ml of Trypsin Neutralizing Solution (CC-5002) [2 ml for each ml of 0.05% trypsin/EDTA (Lonza CC-5002)] is added. The flask contents are transferred to a 15 ml conical tube and this tube is centrifuged at 1000 rpm for 5 min. The tube is sterilized with ethanol and returned to the hood. The supernatant is aspirated, being careful not to disturb the cell pellet. Then, the pellet is re-suspended using fresh pre-warmed Lonza FGM-2 medium and the contents are transferred to the flask/flasks, which were previously filled with Lonza FGM-2 medium. The flasks are gently agitated to make sure that the medium covers the entire bottom surface and then returned to the incubator. Feeding is as indicated above.

B. Skin-Chip Wound Model.

In some embodiments, an open top skin chip as a microfluidic device is used for in vitro simulation of in vivo wound healing. As described herein, for one example, the epidermal cell layer is disrupted. As another example both the epidermal cell layer and dermal layer is disrupted, i.e. wounded, by several means, including by projectiles shot from a tattoo gun, insertion of a tattoo needle, etc. In some embodiments, wounding by a tattoo needle may simulate wounding by an injection needle, such as for insulin injections, vaccine injections, or a microneedle array, e.g. TB tine tests, etc.

As shown herein, a wound created by a tattoo needle healed by day 4 on a skin-chip, visualized by routine histology. Furthermore, wound healing data following a simulated tattoo injury from a needle puncture wound, in one embodiment of a Skin-Chip, showed that wounding induced skin cells to migrate, proliferate and close the wound site. The epidermal cells were observed to undergo stratification to recreate a healthy skin epidermal phenotype. Results of skin wounding shown herein, in FIGS. 10, 12, 14, for examples, were obtained using Skin-chips without endothelial cells. In some embodiments, Skin-chips for use in simulating would healing are without endothelial cells on-chip. In some embodiments, Skin-chips for use in simulating would healing comprise endothelial cells on-chip, preferably located in the spiral channel of an open top device.

Thus, in one embodiment of a skin-chip, skin cells were able to initiate, maintain and turn off a cascade of events involved in wound healing. This wound-healing cascade includes an initial inflammatory response by cells including but not limited to: skin cells and stromal cells (i.e. gel compartment of an open top device), e.g. fibroblasts, macrophages, etc., followed by tissue remodeling. Thus in some embodiments, macrophages are added to a gel compartment of a Skin-Chip. In some embodiments, macrophages are not histocompatibility matched to the other cells on a Skin-Chip.

Therefore, in some embodiments, compounds may be tested to accelerate wound healing of skin. In some embodiments, a compound may be tested in order to determine whether it would delay wound healing of skin.

In some embodiments, skin cells such as keratinocytes, fibroblasts, macrophages, PBMCs, and immune cells, etc., on the same Skin-chip are histocompatibility matched, e.g. derived from the same individual. For example, a shave biopsy or punch biopsy may be obtained from the skin of a person along with a blood sample from the same person for providing skin cells for placement in an open top chip, as part of the intact biopsy or cells derived from the biopsy, including skin cells, i.e. keratinocytes, dermal cells, etc., Langerhans cells, subdermal tissue, hair follicles, melanocytes, and lamina propria, along with the option of adding white blood cells isolated from the blood sample. In some embodiments, a skin biopsy is cultured on top of or with the lumen of an open top chip that may be used for providing a tattoo as described below.

A histocompatibile Skin-chip may be used for testing an individual's response to wounding, in part for identifying an adverse reaction to: a tattoo, tattoo ink formulation, and substances such as alcohol, lidocaine, etc., or response to a substance, such as a sensitization compound, an irritant compound. Thus, a histocompatibile Skin-chip may be used in personalized medicine for use in testing an individual's response, as described herein, in part due to the variability of responses of particles, solutions, substances and compounds, etc., as described herein, because responses in vivo varying from one person to another, i.e. between individuals.

C. Substance/Pigment Testing on Skin-Chips: Safety Testing.

In some embodiments of Skin-Chip experiments, pigments are applied using a tattoo needle inserted into the skin layer on-chip. However, in other embodiments, substances for skin testing may be added to a Skin-Chip by flowing a compound (i.e. substance, including a formulation) through top and/or bottom channels. Further, in other embodiments for other organ-chips, such as kidney, liver, etc., a substance for testing may be added by flowing the substance through top and/or bottom channels.

Exemplary doses of a compound will be specific to each assay, with a treatment period ranging between 3 min up to 48 h, or more. In some embodiments, analysis of effects, such as toxic, phototoxic, etc., will be evaluated by a metabolic activity assay on-chip (e.g. Presto Blue) to evaluate compounds having known ET50 and IC50 values.

In some embodiments, a Skin-Chip will be assessed for recovery after initial damage, e.g. after contact to the epithelial cells in the upper channel, and skin metabolism related to turn over of epithelial cells, in particular. Thus, readouts include assessing recovery after damage. Readouts include but are not limited to Invitrogen PrestoBlue™. Invitrogen PrestoBlue™ Cell Viability Reagent, e.g. Fisher Scientific, Catalog number: A1326, is an exemplary metabolic Assay Reagent. When added to cells, the PrestoBlue® reagent is modified by the reducing environment of a viable cell and turns red in color, becoming highly fluorescent. This color change can be detected using fluorescence or absorbance measurements. Additional examples of viability endpoints include MTT ((3-(4,5-Dimethylthiazol-2-yl)-2,5-diphenyltetrazolium bromide)) assay referring to a colorimetric assay for assessing cell metabolic activity, Neutral Red stain, a live-dead assay, Omics data (e.g. transcriptomic, proteomics, metabolomics); Oxidative Stress (e.g. ROS), etc.

In some embodiments, a live-dead staining is used for determining cell viability. As one example, DRAQ7™ refers to a far-red fluorescent dye that significantly stains nuclei in dead and/or permeabilized cells but not live cells.

1. Systemic Skin Testing.

In some embodiments, an open top skin-chip is used for measuring systemic exposure by flowing a substance, or effluent from a treated Organ-Chip, through the vascular (bottom) spiral channel. In some embodiments, said substance is a drug. In some embodiments, said substance induces a skin reaction in the cells located in the upper part of the open top chip. In some embodiments, a skin reaction is a simulation of a rash observed in vivo in human skin. Such testing is relevant to drug work so is contemplated for using a Skin-Chip for testing for a skin reaction as an indication of a toxic reaction to a drug. Skin often shows signs of a toxicity reaction (like a rash) before other organs do. In some embodiments, drug testing in a Skin-Chip may be used for individualized medicine as described herein.

2. Topical Skin Testing.

As demonstrated herein, one embodiment of a Skin-Chip demonstrates utility for toxicity and safety studies. In fact, a Skin-Chip demonstrates model robustness for topical skin testing including but not limited to for skin barrier tests; skin irritation test; skin photoxicity and photosensitization tests. Topical test compounds, such as cytotoxic, irritant, sensitizer, corrosive, phototoxic compounds are traditionally evaluated through topical application on skin. Therefore, in preferred embodiments, compounds intended for topical application may be applied on top of the cells in the upper chamber of a skin-Chip, i.e. applied through the upper channel or directly onto the skin through the open-top.

MatTek

In some embodiments, topical test compounds, such as positive controls, demonstrated tissue robustness and overall robustness of embodiments of Skin Chips for use in testing other types of topical test compounds. In one embodiment, tissue robustness was evelauated determining the ET50 value following topical exposure to a test irritant, 1% TritonX-100. Irritation potential is calculated in terms of the “ET50” value: the time taken, in minutes, for a test compound to reduce the viability of the skin model to 50% compared with negative controls. In one test, for providing comparative values, a static skin model described herein resulted in a ET50 value around 11 h, compared to skin equivalent models on the market (e.g. MatTek—7 hours). Thus, at least one commercially obtained and tested skin equivalent model became permeable faster than one embodiment of a static skin model described herein.

FIG. 36 shows an exemplary D17 ALI condition. Transwells

TABLE 2A

| | Treatment | | # of | time |
|---|---|---|---|---|
| Model | Group | Concentration | Replicates | points |
| Full- | Caffeine | 3 ug per sample | 3 per time | Effluent |
| thickness | (10 ug/cm²) | (300 ug/mL in | point | sampling at all |
| skin | | PBS - dose | | time points |
| | | 10 ul) | | remaining |
| | | | | donor |
| | | | | receptacle |
| | | | | sampling at |
| | | | | 24 h |
| | | | | time point |

TABLE 2B

Cumulate % of topically applied caffeine

| Model | 1 h | 4 h | 7 h | 24 h |
|---|---|---|---|---|
| Human Skin | 2 | 4 | 6 | 15 +/− 8 |
| Epiderm | 70 | 90 | 92 | 93 +/− 2 |
| (MatTek) | | | | |
| Episkin | 27 | 51 | 56 | 71 +/− 16 |
| (L'Oreal)* | | | | |
| Emulate | 0.6 | 27 | 65 | 102 +/− 2 |

*Dreher et al., L'Oreal Research, 2002

### Skin Irritation—Release of Associated Cytokines.

Topical exposure to a known skin irritant, Triton X-100, shows increase in expression of inflammatory cytokine IL-1B and IL-18, expressed following skin sensitization, shows no significant increase after exposure to Triton X-100, as expected.

### Phototoxicity Assay (Viability).

Safety Assessment of Red122 Pigment—Phototoxicity Assay (Viability) Red tattoo pigments known to cause some level of toxicity following sun exposure.
Static Skin models were exposed to minimal dose of UVA which does not cause tissue damage (5 J/cm2).
Samples tattooed with 35% w/v of Red122 pigment showed some phototoxicity with a decrease in viability to 85% from healthy.
Phototoxicity and Photosensitization to Red122—Cytokines Release Skin tattooed with 35% w/v of Red pigment showed an increased secretion of inflammatory cytokine, but no effect as a skin sensitizer. Following UVA exposure, tattooed samples showed an increase in both cytokines, indicating that Red is both phototoxic and a skin sensitizer under UVA. Chlorpromazine (CPZ), known to be induce phototoxicity and photosensitization, was used as a positive control.

### Phototoxicity to TiO₂ and Blue15—Cytokines Release.

IL-8 is an inflammatory cytokine associated with UVA-induced phototoxicity.

Tattooed samples (no UVA) showed lower baseline expression of IL-8, associated with impaired wound healing caused by to the presence of tattoo pigments.
Skin tattooed with 3% TiO2 and 2% Blue15 showed an upregulation of IL-8 following UVA exposure, in contrast to control and wounded samples which showed no change in IL-8 expression following UVA exposure.

### Future Applications.

Incorporation of dermal filler

Inflammatory properties of dermal fillers

Longevity/degradation of Hyaluronan-based dermal fillers

Young vs Aged cells

Immune cells (resident—Dendritic cells; model in development)

Effect of mechanical forces: stretch on filler degradation and inflammatory response.
Readouts/Methodology: Needle injection (through dermis only or through full-thickness model). Mixed within dermal matrix prior to keratinocyte seeding. Viability (e.g. MTT, Presto Blue, Neutral Red).

Chemokine and cytokine release (effluent); Oxidative Stress (e.g. ROS); Histology; Hyaluronan ELISA; Imaging (fluorescently labeled cross-linkable HA); Oxidative stress (ROS); Hyaluronidase/HAase Elisa (HYAL-1, 2); Histology; Omics data; Barrier function; Collagen levels; Activation markers and cytokines.

Model Platforms: Hybrid static Skin-Chip; Dynamic Skin-Chip; Hybrid static Skin-Chip²⁰¹⁹|Cfidt.

Exemplary positive controls for producing effects include but are not limited to: irritation by TritonX-100, at percentages ranging from 0.128% w/v-1% or more.

Triton-X 0.128% w/v exposure over 24 h; corrosion by Hydrochloric acid (HCL) at 14.4% wt exposure over 3 min; sensitizer by 2-Mercaptobenzothiazole. In some embodiments, quantification of such effects are by measuring IL1-alpha production, e.g. effluent.

In some embodiments, a liquid (solution) comprising a compound for topical skin testing is topically at a volume of 30 ul. In some embodiments, a solid form of a compound to be tested may be dispersed in solution or applied on top of the epidermal layer. In yet other embodiments, a compound may be applied through top-channel exposure, e.g. for topical absorption of compounds, such as a drug. In other embodiments, a compound may be applied through bottom-channel exposure, e.g. to test a skin reaction from systemic exposure, such as a drug, in part because skin often shows signs of toxicity or allergic reactions (e.g. rash) before other organs show signs of toxic effects.

In some embodiments, an open top skin-chip is used for measuring topical absorption of compounds, including identifying adverse effects when a substance is applied through the top-channel. In one embodiment, a substance is a drug, a lotion, a solution, etc. An adverse reaction, as one example, is inflammation, such as observed in vivo as a rash, etc., as described above. In some embodiments, substances are tested as described herein related to tattoos, e.g. creating a tattoo, removing a tattoo, and adverse effects over time following exposure to a compound.

As one example, alpha hydroxy acid (AHA) compounds (substances), including but not limited to citric acid, lactic acid and glycolic acid, are commonly used for topical treatments of skin, e.g. dry skin, sun damaged skin, acne, wrinkled skin, etc., including in many types of cosmetics. Alpha hydroxy acids may also be used for deliberately inducing skin peeling, e.g. facial peels. Facial peels in particular often contain 10% to 70% glycolic acid in one application. Depending upon the concentration of AHAs, length of time in contact with the skin, and sensitivity of the skin, facial peels may cause moderate to severe skin irritation, redness, and burning. Facial peels left on the skin for periods longer than recommended can cause severe burns to the skin, in part removing the top layer of live skin cells. AHAs in general may cause mild skin irritation, redness, swelling, itching, and skin discoloration.

Thus, a Skin-Chip was treated with one of citric acid, lactic acid and glycolic acid, in order to determine each acid's effect on skin cells in a Skin-Chip, both initially and during a one day exemplary recovery period.

FIG. 64A shows exemplary evaluation of a compound's toxicity to epidermal cells in a Skin-Chip after a 15-minute exposure, by DRAQ7™ staining. Citric acid (0.6M), lactic acid and glycolic acid were each tested on a Skin-Chip. At the concentrations added, each of the three acids showed statistically significant death of skin cells in the chip with glycolic acid showing the highest cell death, dead cells/mm². Insert shows highly florescent dead cells.

FIG. 64B shows exemplary evaluation of a compound's effect upon metabolic activity measured by PrestoBlue™, as a percentage of duplicate control-untreated skin-chips. On Day 0 (D0), each of the three acids shows a reduction in activity compared to control (100%). However glycolic acid shows a statistically significant reduction in activity compared to the activity of citric acid (0.6M) and lactic acid. By Day 1 (D1), remaining cells treated with each one of the three acids shows a higher metabolic activity as they are recovering, than on D0, while citric acid metabolic activity recovery was statistically significantly higher than lactic acid and glycolic acid exposed cells.

D. Tattooed Skin Chips.

In some embodiments, an open top skin-chip as one embodiment of a microfluidic device is used for in vitro simulation of creating a tattoo, observing the tattoo over time, or removing a tattoo in vivo, e.g. in one or more locations, including tattooing on top of the epidermis, within the epidermis and dermis and within the dermis, etc. In some embodiments, a tattoo on a skin-chip is created by opening the top of a mature skin-chip, i.e. a skin-chip comprising skin cells having a keratinized layer of epidermis on top of a maturing epithelial layer where dividing epithelial cells are located on top of a dermal layer, in variations such as with or without a stromal area, with or without an endothelial layer in the lower channel, e.g. spiral lower channel, then using a tattoo gun and/or tattoo needle as a means for applying a substance onto or into skin cells. In some embodiments, a substance may be a fluid, e.g. a fluid used in a tattoo ink, a diluent used for a tattoo ink, a diluent used for a pigment, etc. In some embodiments, a substance may be a pigment and/or contain a pigment, for example a tattoo ink formulation, a pigment used in a tattoo ink formulation, etc. In some embodiments, a substance may contain a potentially toxic compound found in tattoo ink.

In some embodiments, a dilute is a mixture of substances, such as shown in Table 3. In some embodiments, a dilute is Witch Hazel. In some embodiments, a dilute is an alcohol.

1. Tattoo Dose (TD).

In one embodiment, for testing compounds, such as tattoo inks, tattoo pigments, and metals found in tattoo inks, On-Chip, in vitro doses relevant to in vivo exposures were formulated using ECHA (European Chemicals Society)—REACH information. ECHA refers to the European chemical agency that releases REACH standards. REACH (Registration, Evaluation, Authorisation and Restriction of Chemicals) refers to a regulation of the European Union including suggestions for regulation. REACH suggests limits on exposure to chemicals, including chemicals contained in consumer goods sold in Europe.

In fact, ECHA's Committee for Socio-economic Analysis (SEAC) published an opinion suggesting concentration limits on substances including carcinogenic, mutagenic and reprotoxic (CMR) substances, skin sensitizers or irritants, substances corrosive or damaging to the eye, metals as well as other substances regulated in cosmetic products. Their goal is to make inks for tattooing safer and protect people from serious health problems or effects of substances contacting the skin. Thus, Echa Response to comments document (RCOM) (ECHA/RAC/RES-O-0000001412-86-240/F, dated 29 Nov. 2018 and last modified Jan. 2, 2019), lists some substances used in tattoo inks and permanent make-up and proposes concentration limits for exposures, including but not limited to: Cadmium (0.00002% w/w), Cobalt (0.0025% w/w), Lead (0.00007% w/w), etc.

Furthermore, specifically related to tattoo inks and permanent make-up (PMU), substances used in coloring belong to several groups. Up to 60% w/w of inks but typically around 25% are coloured molecules, including colourants, referring to colored pigments (e.g. metal salts, plastics, vegetable dyes, typically poorly soluble in water), lake pigments (e.g. a soluble dye precipitated with an inert binder, usually a metallic salt), dyes (organic molecules that are typically soluble in water, e.g. colorant that is dissolved and suspended in liquid). Other groups include impurities, and other auxiliary ingredients, including solvents, stabilisers, “wetting agents”, pH-regulators, emollients and thickeners.

ECHA reported that an average estimate pigment load per tattoo is 3.59 mg/cm2 (75th percentile). A range of pigment exposure from a tattoo is 0.60-9.42 mg/cm². See, Table 9 in ECHA, ANNEX XV RESTRICTION REPORT, VERSION NUMBER: 1.2, October 2017. Thus, in some exemplary embodiments, a dose of ink may be chosen from a range of 0.01 to 3.4 mg/cm2 of ink, such as from a tattoo ink sample, or a potentially toxic or known toxic compound used in ink, e.g. TiO2.

For exemplary skin tattoo experiments, Skin-chips were tattooed with concentrations of 0.01-3.4 mg/cm² of TiO2 as a “Tattoo Dose.” For systemic chip experiments, doses range from “0.003-0.24% TD” for the Liver- and Kidney-Chips. However, the concentrations used herein, are specific to TiO2, unlike the ECHA concentrations for pigment load that is general to tattoo pigments. Thus, values used herein are for biodistribution into the liver-chips and kidney-chips are specific to TiO2, which was selected as an exemplary and nonlimiting agent (i.e. substance, compound).

As one example of biodistribution, “TiO2 In Vivo Biodistribution—Subcutaneous Route”, Kessler et al, Nature 2017, implied systemic movement of TiO2 after a 12 mg topical treatment of skin, TiO2 topical treatment of burn wounds, resulted in 2 ug/g per organ weight in liver (0.022% of treatment dose) and 0.3 mg/g in kidney (0.00045% of treatment dose) (see, FIG. 18, table on the right).

However, it is not meant to limit doses of agents/substances to the examples provided herein. Moreover, while the biodistribution data, shown in FIG. 18, may be known for TiO2, there is little information or data known for the majority of substances found in tattoo inks or other types of compounds applied to the skin. Therefore, a Skin-Chip is contemplated for use in order to test how much of the ‘agent’ applied to the Skin-Chip, either topically or systemically, would make it into blood (i.e. a vascular channel's output/effluent). Once this amount is established, it will be used to determine test exposure amounts applied directly to Liver-Chips, Kidney-Chips, etc.

However, the amount of agent released by a Skin-Chip effluent as an example, may or may not reflect amounts that would actually reach other organs via blood in vivo. In particular, because it is contemplated that no one-to-one relationship exists between applied amounts to the skin for providing an exposure to another organ in vivo, extrapolations of in vitro measurements may be needed in order to simulate in vivo amounts reaching an organ. In vivo amounts vary in part, since the agent's concentration in blood either downstream and/or recirculated, is a function of the applied amount reaching the blood (e.g. by skin absorption), in addition to the rate of clearance (e.g. through the liver or kidneys) and the agent's volume of distribution in the body (which relates to other factors including but not limited to the degree of ability of a chemical compound to dissolve in fats, i.e. a lipophilic compound).

2. In Vivo Simulation of an In Vitro Immune Response: Phagocytosis of Foreign Particles.

Carbon Black particles, fluorescent microbeads, etc., may be used to assess potential particle size effect of biological response, i.e. cellular uptake by dermal fibroblasts.

Thus, in some embodiments, a Skin-Chip Replicates Physiological Response to Tattoo Cellular Uptake of Foreign Particles and Immune-Response. Fibroblasts contribute to tattoo permanence by engulfing foreign particles. Fibroblasts actively uptake and retain tattoo pigments and fluorescent particles of all size ranges that were tested. In some embodiments, motile macrophages are transporting foreign particles; Carbon Black and Fluorescent Microbeads. Macrophages phagocytosed Beads.

In some embodiments, macrophages with skin epidermal keratinocytes in traditional plate culture were used for pre-chip experiments.

In some embodiments, Immune Cell (Macrophage) Activation was observed Within Tattooed Area. Immune cells cluster within tattooed region via inflammatory response (CD80+showing an activated pro-inflammatory phenotype—M1).

In some embodiments, Skin-Chip Replicates Physiological Response to Tattoo Injury: Skin Wound Healing, including as described above. The Skin-Chip demonstrates for the first time wound healing capabilities in an in vitro human skin model, recapitulating physiological response to tattoo injury through a cascade of immune response, cell activation and migration, and ECM remodeling. Skin-Chip is able to heal through epidermal and dermal remodeling of wound site.

In some embodiments, Tattoo on Skin-Chip: Pigment Shedding through the Epidermis. Epidermal and dermal remodeling during wound healing show trapped pigment within epidermis, which become isolated and pushed upwards as the epidermal cells go through their cycle of stratification towards to upper most layer of the skin (normal skin cycle=14 days). Epidermal and dermal wound closure observed by keratinocyte migration from the wound edge and dermal contraction.

In some embodiments, a skin-chip was used for safety testing of metal containing particles, e.g. TiO2 particles. Levels and descriptions are provided for TiO2 in Vivo Biodistribution—Subcutaneous Route Nature 2017, Kessler et al.

Modeling Systemic Exposure Assessment of White Pigment (TiO2) Toxicity on the Skin.

Toxicity in the Skin-Chip: TiO2 Tattooed on Skin-Chip. Dose-dependent response 24 h post-tattoo observed by a decrease in viability with increasing TiO2 concentration. Skin recovery observed over 7 days, except at highest dose leading to tissue necrosis.

Assessment of Tattoo Pigment Toxicity (TiO2, Blue15, Red122) on the Skin, Liver, and Kidney-Chip.

### II. Chips for Determining Systemic Effects: Systemic Safety Testing.

In vivo, ‘agents’ can go from the skin into the body and other organs both by means of blood and lymph. As illustrated in FIG. 1B, agents applied to the skin may distribute in numerous areas within the body. Thus, in some embodiments, Skin-Chips and other Organ-Chips may be used for modeling systemic distribution of substances applied to the skin. In some embodiments, Liver-Chip and Kidney-Chip exposure to a substance is through flow. In some embodiments, a test agent, includes but is not limited to an ink, pigments, etc., and may be used for testing other types of agents, such as found in cosmetics, hair dyes, etc. In some embodiments, a test agent is administered into the vascular channel, as a simulated exposure physiologically through the blood. In some embodiments, a test agent is administered into the top channel as a simulated exposure physiologically through the epithelial cells, e.g. for the Kidney-Chip. In some embodiments, a test agent is administered into both the vascular channel and the epithelial channel.

Thus, in some embodiments, Organ-Chips may be used for Modeling Systemic Exposure Assessment of White Pigment (TiO2) Toxicity on the Liver-Chip, and Kidney-Chip, etc. In some embodiments, a closed top microfluidic S1 chip device, e.g. liver-chip, lymphoid-chip, kidney-chip, BBB-chip, brain-chip, lung-chip, etc., as embodiments of a microfluidic device is used for testing toxicity of tattoo pigment particles. In some embodiments, a double membrane chip e.g. brain-chip, innervated brain-chip, lymphoid-chip, etc., is used for testing toxicity of tattoo pigment particles. In some embodiments, pigment particles are added directly to a microfluidic device for testing toxicity of a type of particles, e.g. a metal particle, i.e. Tio2, etc.

Pigment with a low concentration of 0.03% of pigment diluent is flowed through the chip (same concentrations both top and bottom channels simultaneously). No macrophages were included in this study. In future applications, we also plan to explore potential toxicity of pigment diluent formulations (without pigments). There was no static period for the pigment to settle, but due to the low viscosity and surfactant concentration of the final diluent concentrations, pigments settled quickly even under flow.

In some embodiments, test agents include pigments, pigment diluent, (i.e. one or more of substances found in an ink formulation), etc. Exemplary diluent substances are provided in Table 3. Diluent used herein was provided from Intenze and consists of the following ingredients, See Table 3.

TABLE 3
Substances Contemplated for Testing in microfluidic devices.

| | | | Provided by |
|---|---|---|---|
| Reagent | Use or Function | Concentration | Intenze |
| Glycerin | — | 3-5% | Yes |
| Propylene | — | 3-5% | Yes |
| glycol | | | |
| Witch | Astringent | 25% | No |
| Hazel | (contraction); Reduce | | |
| | skin inflammation | | |
| Steol | Surfactant | 0% in formulation | Yes |
| | (widely used in some | provided by | |
| | tattoo formulations) | Intenze due to | |
| | | adverse results in | |
| | | animal testing | |
| | | (Planterin is used | |
| | | instead in Intenze | |
| | | formulation) | |
| Syntran | Acrylate polymer to | — | Yes |
| | stabilize pigment (color | | |
| | ink only; not present in | | |
| | black ink) | | |
| Planterin | Surfactant | 0.01-0.04% | Yes |
| | (added at the end) | | |

Assessment of Tattoo Pigment Toxicity (TiO2, Blue15, Red122) on the Liver-Chip. Both top and bottom channels were treated

Treatment Groups Concentrations Endpoints

A. Skin-Chips:

During the development of the present inventions, several embodiments of Skin-Chips were provided for use as an in vitro repair model of full-thickness epidermis using both static chip devices and microfluidic chip devices. These full-thickness Skin-Chip devices were used to demonstrate full-thickness epidermal skin wounding followed by skin repair, in part assessed by tattoo pigment uptake by skin cells (dermal and epidermal). In some embodiments, added immune cells (e.g. MV4-11 macrophage-like cancer cell line) showed spontaneous activation following tattoo ink deposition or needle injury.

Assays to evaluate skin irritation, phototoxicity, and photosensitization with and without the presence of tattoo pigments in the skin were optimized using exemplary chips.

Viability: Within the first day following tattoo injury, skin viability shows a declining trend with increasing pigment concentrations. However, tissue recovery was observed over 7 days post-tattoo for all pigments tested.

FIG. 65 shows exemplary 4 days post-tattoo wound closure.

Phototoxicity and Photosensitization: Red122 shows both cytotoxicity and sensitization following UVA exposure (observed by expression of cytokines IL-1Beta and IL-18); TiO₂ and Blue15 show UVA-induced upregulation of cytotoxic marker IL-8

Wound repair: Presence of tattoo pigments shows impaired wound healing via reduced release of ECM remodeling enzyme MMP-9 and pro-inflammatory cytokine IL-8.

Barrier quality: Exposure to TritonX-100 shows improved barrier quality compared to competition such that the epidermal barrier resisted effects of this exemplary irritant (ET50 of 11 h vs 7 h MatTek), and shows that model can be utilized to assess skin irritancy via quantification of irritant-associated cytokine release (IL-1Beta).

In some contemplated embodiments, CBD (Hemp extract) permeation through the skin will be evaluated to assess, in part, potential systemic toxicity and functional change to the liver and kidney. Two exemplary formulations may be tested: Formulation #1 nano-emulsified CBD distillate and Formulation #2 MCT/CBD oil dilution (medium chain triglyceride).
Assess degradation rate of Hyaluronic Acid fillers within the skin over up to 1 month in culture
Pilot test will include two fillers: Filler #1 degradation profile within 1 week, Filler #2 degradation profile within 1 month
Filler degradation will be assessed using the following approaches:

Emulate
Histological staining (Alcian blue) Merz
MicroCT of skin samples
effluent analysis for detection of degraded HA (turbidity assay and Carbazole assay)
SOW draft: https://app.box.com/s/j3lwu70 h5blq7tlamhs9ei0tlwbwrlfn
Next steps: interested in effect of stretch on degradation profile

Next Steps and Other Applications
Ongoing optimization of Zoe-compatible Open-Top Chip V2
Successfully maintained chips over 2 weeks in culture at ALI without gel delamination or contraction
Repeat assessment of barrier quality (Triton-X MTT assay, caffeine permeability) and histological

characterization (immunofluorescence, H&E) to compare with transwell model
Include readouts of skin metabolism (glucose consumption and lactate production)

B. Liver Chips.

In some embodiments, a Liver-Chip microfluidic device is a closed-top S1 device as described herein. In one embodiment, a Liver-Chip is contemplated as an Open-Top Chip, as described herein.

Embodiments of Liver-Chips include: Co-culture (Hepatocytes+LSECs); Tri-culture (Hepatocytes+LSECs+Kupffer cells); Quad-culture (Hepatocytes+LSECs+Kupffer cells+Stellate cells); Human Liver-Chip; Rat Liver-Chip; Dog Liver-Chip; etc.

Readouts include but are not limited to: albumin secretion, Effect of Bosentan on Albumin Secretion; Inhibition of BSEP Transporter; Bile acid accumulation Cholyl-lysyl-fluorescein (CLF); BSEP substrate etc.

Demonstrated species differences in the hepatotoxicity at in vivo relevant doses of Bosentan were identified. For one example, albumin data shows dose response effects for dog and human, but not for rat, which correlates with in vivo data: human>dog>rat.

Day 6 Post Exposure to TiO2: LDH Leakage & Albumin Secretion. No significant cell damage (LDH leakage) observed following exposure to TiO2. No significant change in cell function (albumin secretion) following exposure to TiO2.

### Liver-Chip

FIG. 40A shows an exemplary schematic embodiment of a Liver-Chip for assessing pigment toxicity in the Liver-Chip. Left panel, J Clin Invest. 2007; 117(3):539-548.

FIG. 40B-shows exemplary schematic embodiments of a Liver-Chip as a microfluidic device which may find use with the present invention.

FIG. 41 shows one exemplary schematic embodiment of a quad Liver-Chip 1-8.

FIG. 42 shows exemplary readouts for assessing pigment toxicity in the Liver-Chip. Readouts include but are not limited to albumin secretion and transporter studies, e.g. CLF as a BSEP transporter substrate for showing bile acid accumulation.

FIG. 43 shows an exemplary timeline and experimental variables.

FIG. 44 shows exemplary Liver Damage at Day 6 Post Exposure to TiO₂: LDH Leakage & Albumin Secretion.

Liver Hepatocytes left LDH Leakage chart, Endothelial Cells (LSEC), LDH Leakage in Liver Endothelial Cells (LSEC) shown in the right LDH chart. Injury to liver function is indicated by increases in albumin Secretion.
No significant cell damage (LDH leakage) observed following exposure to TiO₂.
No significant change in cell function (albumin secretion) following exposure to TiO₂.
Other functional changes or mechanistic routes may be detectable through additional assays

FIG. 45 shows one exemplary Hepatocyte Cell Morphology (upper 6 panels) and nonparenchymal cell (NPC) Morphology (lower 6 panels) after 4 Days of Treatment with a range of concentrations of Blue15 comparing to staurosporine (5 microM) as a positive control showing hepatocyte damage. Black arrow points to gaps in cell coverage indicated a loss of cells.

FIG. 46 shows one exemplary embodiment of a Liver-Chip demonstrating albumin secreted over time measured in effluent from the top channel. A range of concentrations of Blue15 was tested comparing to staurosporine (5 microM) as a positive control showing a loss of secreted albumin.

FIG. 47 shows one exemplary Hepatocyte Cell Morphology (upper 6 panels) and nonparenchymal cell (NPC) Morphology (lower 6 panels) after 4 Days of Treatment with a range of concentrations of Red122 comparing to staurosporine (5 microM) as a positive control showing hepatocyte damage. Red arrow points to gaps in cell coverage indicated a loss of cells.

FIG. 48 shows one exemplary embodiment of a Liver-Chip demonstrating albumin secreted over time measured in effluent from the top channel. A range of concentrations of Red122 was tested comparing to staurosporine (5 microM) as a positive control showing a loss of secreted albumin.

FIG. 49 shows one exemplary embodiment of a Liver-Chip demonstrating effects of Red122 & Blue15 on Liver Function via ATP Synthesis as measured from the effluent collected from the bottom channels. A range of concentrations of Red122 (left) & Blue15 (right) were tested comparing to staurosporine (5 microM) as a positive control showing a loss of ATP synthesis.

Pigment uptake: All cell types (hepatocytes, stellate cells, Kupffer cells) showed uptake of tattoo pigments.

Cell morphology: Pigments did not have any significant effect on cell morphology.

Cytotoxicity: No significant cell toxicity observed via LDH leakage.

Cell functionality: No significant change in hepatocyte function observed via albumin production and ATP levels.

C. Kidney-Chips.

In some embodiments, a Kidney-Chip microfluidic device is a closed-top S1 device as described herein. In the kidney-chip, parenchymal cells can include cells of collecting tubules, the proximal and distal tubular cells, and any combinations thereof.

Readouts include but are not limited to: Gentamicin-induced toxicity after exposure to 10 mM of gentamicin for 48 hours. Microscopic analysis of the proximal tubular epithelium shows structural damage coupled with significant increase in LDH, ROS, and NAG in medium effluent and increase active caspase-3 in cells lysates.

Day 6 Post Exposure to TiO2: Kidney Epithelial Cells Morphology.
Severe morphological change coupled with cell detachment observed following Cisplatin treatment (positive control).
No significant morphological change observed with TiO2 exposure.

Day 6 Post Exposure to TiO2: LDH Leakage & NAG Activity
No significant cell damage (LDH leakage) observed following exposure to TiO2.
NAG activity showed no toxicity at lower dose, but some effect at the higher concentration of 0.24% TD (Tattoo Dose).
No significant cell damage (LDH leakage) observed following exposure to TiO2.
   NAG activity showed no toxicity at lower dose, but some effect at the higher concentration of 0.24% TD.

Assessment of Tattoo Pigment Toxicity (TiO2, Blue15, Red1122) on the Kidney-Chip.

TABLE 4

| Treatment Groups | Concentrations |
|---|---|
| Untreated | N/A |
| Cisplatin | 30 micro M |
| (induce cell death) | |
| Vehicle: Intenze Ink Diluent | 0.03 v/v |
| TiO2 | 0.003-0.24% Tattoo Dose |

| | | | |
|---|---|---|---|
| | 0.06% | | |
| | | 0.1% | DMSO |
| | | 10-30 [symbol] | |
| | 0.003-0.24% | (0.004-0.04%) | 0.0012-0.0108% |
| | Dose 0.06% | | Endpoints• |
| | | | Morphology |
| (bright-field)• | function | and | cytotoxicity |
| Kidney | | | |
| (ALP, | NAG | activity, | LDH, |
| ROS, | Caspase) | | |
| | Blue15Red122 | | |
| | 0.0018-0.021% | | |

### Tattooed

Channel: Renal Proximal Tubule Epithelial Cells

Bottom Channel: Renal Microvascular Endothelial Cells

### Endpoints

Morphology: BF Imaging From Effluent: LDH, ROS

Terminal Endpoints From Lysates: Caspase, ALP

Day 6 Post Exposure to TiO2: Kidney Endothelial Cells

Day 6 Post Exposure to TiO2: LDH Leakage & NAG Activity

Morphology: Red Dye, Day 7 Post-Dosing

Significant morphological damage was observed in the Cisplatin treated group, however, no significant differences were noted

between the dye treatments. Clusters of dye particles were noted at the highest dose of blue and red dye tested.

Red122 and Blue15: Effect on Kidney Toxicity Via LDH Release
Lactate dehydrogenase (LDH) is an enzyme present in all cells and released upon cell membrane damage.
Quantification of kidney damage via LDH release

No significant changes in LDH were observed between the different doses of Red and Blue tested

Red122 and Blue15: Effect on Kidney Toxicity Via Caspase
Caspase is a critical mediator of programmed cell death (apoptosis).
Quantification of caspase expression correlates with cell death.

No significant differences were noted in caspase activity between the different doses of red and blue tested

Red122 and Blue15: Effect on Kidney Toxicity via ROS Activity

Reactive Oxygen Species (ROS) release was quantified as a measure of kidney toxicity.

Accumulation of ROS leads to DNA, RNA, and proteins damage, as well as cell death.

No significant differences were observed in the ROS activity were observed between the different groups

Red122 and Blue15: Effect on Kidney Function Via ALP Activity

Alkaline phosphatase (ALP) is an enzyme that helps break down proteins and is associated to both liver and kidney function.

Decrease in ALP activity is associated with impaired kidney function.

No significant differences were observed in the ALP activity for different doses of red and blue dye tested.

D. Kidney-Chips:

Pigment uptake: Renal microvascular endothelial cells showed uptake of tattoo pigments.

Cell morphology: Of all 3 pigments, only TiO2 showed morphological change in kidney endothelial cells, with a declining trend in the quality of cell morphology with increasing pigment concentration.

Cytotoxicity: No significant changes were observed between the different concentrations of all 3 pigments for all the endpoints analyzed (LDH, Caspase, ROS).

Cell functionality: No significant changes in kidney function and metabolism observed for all pigments measured by ALP and NAG activity.

### Renal-Kidney Proximal Tubule-Chip

FIG. 52A shows an exemplary embodiment of a Renal-Kidney Proximal Tubule-Chip, as one embodiment of a microfluidic device, for assessing pigment toxicity using exemplary biomarkers as shown.

FIG. 52B shows exemplary embodiments of a Renal-Kidney Proximal Tubule-Chip for assessing pigment toxicity and demonstrating types of exemplary readouts of toxicity. Gentamicin treatment is used for inducing damage as a positive control compared to controls without Gentamicin. Such readouts include but not limited to biomarkers, morphology differences and physiological differences, such as demonstrated by changes in observed morphology, LDH activity, Caspase activity, NAG activity and Reactive Oxygen Species (ROS) activity.

FIG. 53 shows exemplary embodiments of methods for providing Renal-Kidney Proximal Tubule-Chip experimental timelines for using Renal-Kidney Proximal Tubule-Chip when assessing compound toxicity, e.g. pigment toxicity, dye treatment, tattoo inks, in addition to ink diluent or other nonpigment compounds used or found in tattoo inks, toxic compounds used or found in tattoo inks, cosmetic compounds.

FIG. 54 shows exemplary Kidney Proximal Tubule-Chip at Day 6 Post Exposure to TiO₂ (0.003%, 0.05% and 0.24% TD): Kidney Epithelial Cells Morphology. Severe morphological changes are observed following 30 microM Cisplatin treatment (positive control). No significant morphological change observed with TiO₂ exposure. Blue arrows point to examples of cell detachment in Cisplatin treated chips. Pink arrows point to examples of pigment aggregates in TiO₂ treated chips.

FIG. 55 shows exemplary microscopic images of Kidney Proximal Tubule-Chip morphology on Day 6 Post Exposure to TiO₂. An exemplary microscopic image of endothelial cells after they were treated with 0.24% TD TiO2 nanoparticles. Nanoparticles (black) can be seen internalized in the endothelial cells surrounding the nucleus (oval and circular clear areas).

FIG. 56 shows exemplary Kidney Proximal Tubule-Chip: Assessment of Toxicity via Morphological Score showing a poor cell morphology rating on Day 6 Post Exposure to TiO₂. Observed trend in decline of endothelial morphological quality with increasing concentration of TiO₂. The epithelial layer was not evaluated due to pigments covering the cell monolayer. Morphological Score provides a rating of the quality of cell morphology assessed via morphological scoring. High score correlates with poor cell morphology.

FIG. 57 shows exemplary Kidney Proximal Tubule-Chip epithelial cell damage at Day 6 Post Exposure to TiO2: LDH Leakage & NAG Activity.

No significant cell damage (LDH leakage) observed following exposure to TiO₂. NAG activity showed no toxicity at lower dose, but some effect at a higher concentration of 0.24% TD. Additional assays may detect other mechanistic levels of kidney toxicity.

FIG. 58 shows exemplary Red122 and Blue15 effects on morphology and growth.

FIG. 59 shows exemplary Red122 and Blue15

FIG. 60 shows exemplary Red122 and Blue15: Effect on Kidney Toxicity via LDH Release and Caspase induction.

FIG. 61 shows exemplary Kidney Proximal Tubule-Chip pigment ROS.

FIG. 62 shows exemplary Kidney Proximal Tubule-Chip pigment caspase and ALP.

Brief Summary of Pigment Safety Testing on Skin-Chips, Liver-Chips, and Kidney-Chips.

In some embodiments, Intenze inks are used as exemplary pigments for safety testing. In some embodiments, TiO2, Red122, Blue 15 are used as exemplary pigments for safety testing. Physiological viability of skin cells was measured (e.g. Presto Blue). Physiological viability in metabolic/functional assays for the kidney and liver-chip (e.g. Presto Blue). Toxicity assessment of at least 3 pigments (e.g. TiO2, Red122, Blue 15) injected into the skin-chips, or added to kidney-chips, and liver-chips.

### Exemplary Kidney-Chip Protocol.

Exemplary Materials are briefly described as follows.

ECM-coating. Sulfo-sanpah (Covachem, #13414), ER1 (0.5 mg/ml) in ER2 50 mM HEPES buffer; Collagen IV (BD Corning, 50 μg/mL in Dulbecco's phosphate-buffered saline (DPBS); and Matrigel (BD Corning, reduced growth factor, 100 ug/ml DPBS).
Cells. Top channel. Human Proximal Tubular Epithelial Cells (Lonza, RPTEC #CC-2553); and Bottom channel. Primary Human Glomerular microvascular Endothelial cells (Cell Systems. ACBRI 128), expand to P7 (e.g. passage 7).
Media. Renal Epithelial Growth Medium (REGM™ Lonza, CC-3190) for Proximal tubule cells or REGM2 (from PromoCell, Cat #C-26130); and Kidney endothelial cell medium (Cell Systems, 4ZO-500).
Chip. High shear chip; under flow shear; and Tall channel closed top-chip.
Experimental reagents. Collagen IV coated 6 well plate; and Corning BioCoat Collagen IV multiwall plates, Corning #354428.

One brief exemplary timeline is described as: Day −2: Chip coating; Day −1: Seeding endothelial cells; Day 0: Seeding proximal tubule epithelial cells; Day 0-7: Maintain chips; Day 7: Start Experiment (Study), e.g. 72 hours; and Day 10: End 72 hour Experiment (Study). Exemplary readouts include but are not limited to: Phase contrast microscopic images; immunofluorescent images; barrier function (in particular for kidney-chips, etc.); and Troponin I release (in particular for heart-chips, i.e. cardiac-chips).

A more detailed exemplary timeline, e.g. (proximal-tubule) Kidney-chip is described below.

### Day 0: Chip Activation and Coating

1. Wash the top and bottom channels with 200 μl of 70% ethanol each channel.
2. Aspirate the fluid from both channels.
3. Wash both channels with 200 μL of sterile water each channel.
4. Aspirate the fluid from both channels.
5. Wash both channels with 200 μl of ER2 buffer each.
6. Add working solution of ER1 (0.5 mg/ml final concentration, 5 mg ER1/10 ml ER2) to top (50 ul) and bottom (20 μl) channels.
7. Activate the channel with UV light for 20 min.
8. Gently aspirate ER1 from the channels.
9. Wash both channels with 200 μl of ER2 each.
10. Wash both channels with 200 μl of PBS each.
11. Aspirate PBS from both channels gently.
12. Add ECM in PBS (Collagen IV (50 μg/ml)+Matrigel (100 ug/ml)) to top (50 μl) and bottom (20 μl) channels of a standard S-1 closed top chip. In one contemplated embodiment, a high shear chip may be used with 15 μl each for top and bottom channels.
13. Incubate the chip at 37° C. overnight.
   Next day, gently wash the channel with endothelial media.

### Day 1: Endothelial Cell Seeding

1. Expand kidney Glomerular endothelial cells for 2-3 days.
   Add 5 ml of attachment factor to T75 flask and leave at least 5 seconds.
   Aspirate the attachment factor and add 20-30 ml of fresh growth media to flask and incubate at 37° C. until media is at 37° C.
   Thaw a frozen vial of cells at 37° C. in a water bath and immediately transfer the cells into a conical tube containing 14 ml of cold growth media.
   Centrifuge the cells at 900×g for 10 min at 4° C.
   Gently aspirate the supernatant.
   Resuspend the cells in 2 ml growth media and add the cells into T-75 flask.
   Culture the cells at 37° C. for 2-3 days.
2. On day of cell seeding, trypsinize the cells and spin at 900×g for 10 min at 4° C.
3. Count the cells and make 5×10\hat{ }6 cells/ml density for a tall channel chip and seed 20 μl for bottom channel. For a high shear chip, dilute cells at 10×10\hat{ }6 cells/ml then add 10 ul of cells into bottom channel. Final cell concentration is 100,000 cells/chip.
4. Flip the chip and incubate for 90 minutes (min) at 37° C. in an incubator.
5. Add media on top of the inlet and outlet port, gravity washing and feeding.
6. Incubate for 1 day.
7. Prior to proximal cell seeding, stop flow using tips for bottom channel.

### Day 2: Proximal Tubular Cell Seeding

1. Expand Human Primary Proximal Tubular cells in 6-well plate (Collagen IV coated) for 3-4 days.
   Coat 6 well plates with Collagen IV (50 μg/ml)/Matrigel (100 μg/ml) for at least 2 h at 37° C., or use a Col IV coated plate (e.g. Corning #354428).
   Wash with Dulbecco's phosphate-buffered saline (DPBS) and seed Renal Proximal tubular cells at 180,000 cells per well (20,000 cells/cm²).
   Culture for 3-4 days.
2. Trypsinize the cells and count
3. Make 2×10\hat{ }6 cells/ml for tall channel chip and seed 40 μl into top channel. For high shear chip, make 8×10\hat{ }6 cells/ml density and seed 10 μl of cells into top channel. Final cell concentration is 80,000 cells per chip.
4. Incubate for 90 min at 37° C. incubator.
5. Add media REGM on top of the inlet and outlet port, gravity washing and feeding using tips.
6. Incubate for 1-2 days static (i.e. no flow).
   Day 4: Start Flow at 30 ul/hr.
1. Warm media degassing using steriflip for 15 min at 37° C. bead bath.
2. Incubate the media at 37° C. in an incubator after loosening the cap, i.e. unscrewing the cap a bit, but not enough to allow contamination of the media, to ensure gas equilibration.
3. Add 3 ml media in Inlet port and 0.3 ml in Outlet port Reservoir.
4. Prime the perfusion manifold in the culture module.
5. Connect the chip to the perfusion manifold and start flow.
6. Change media every other day.
7. Culture for 6-7 days.

### Day 7-10: Nephrotoxin Testing and Readouts.

Outflow from chips, (e.g. S1—closed top chip; and high shear (HS) chip) was collected for certain readouts. For reference, kidney endothelial media contains 5% FBS while the kidney epithelial media contains 0.5% FBS.

Read outs include but are not limited to: a Kidney injury panel from MSD (K15189D, K15188D); Kidney gene expression: transporters (MRP2, 4, MDR1, MATE1/2-K, OAT1, OAT2, OAT3, OATP4C. OCT2, MRP1/3/5/6, etc.); Immunostaining: antibodies (MRP2, 4, MDR1, MATE1/2-K, OAT1, OAT2, OAT3, OATP4C, OCT2, MRP1/3/5/6, etc.)

C. Lymph Node Chips (Lymphoid Tissue-On-Chip).

A fluidic lymphoid tissue-on-chip may be used for detecting systemic effects of wound healing and exposure to foreign bodies in the skin. Thus in one embodiment, a skin-on-chip is fluidically connected to a Lymph Node-on-Chip. Alternatively, in one embodiment, effluent collected from a skin-on-chip is flowed into to a Lymph Node-on-Chip.

In some embodiments, effluent may be directly used without dilution or modification. In some embodiments, effluent may be modified, including but not limited to filtration, centrifugation, e.g. to remove cellular debris, etc. In preferred embodiments, a lymphoid tissue-on-chip mimics at least some functions of the human lymph node and/or human lymphoid tissue. The device can be seeded with cells from human blood and lymphatic tissue (or cells derived from or related to these cells), include an extracellular matrix for the development of immune system components, optionally allow for the application of mechanical forces (e.g., the pressure of lymph moving from the arm into the lymph node), and provide for the perfusion of fluids and solids resembling blood and lymphatic fluid within fluidic channels.

Parenchymal cells seeded into Lymph Node Chip include but are not limited to reticular cells, blood cells (or precursors to blood cells) such as lymphocytes, monocytes, plasma cells, macrophages, and any combinations thereof.

Additional information is provided in PCT/US2017/042657, HUMAN LYMPHOID TISSUE-ON-CHIP, filed 18 Jul. 2017 and published as WO/2018/017605 on 25 Jan. 2018, herein incorporated by reference in its entirety.

FIG. 51 shows one exemplary embodiment of a Lymph Node-Chip for assessing pigment toxicity, as one embodiment of a microfluidic device that may find use with the present invention. Micrograph shows a close up image of Jurkat cells and microfabricated traps that comprise the Lymph-Node Organ Chip.

E. Other Organs, One Example: BBB-Chip/Brain-Chip.

In some embodiments, a BBB-Chip/Brain-Chip is contemplated for use in indenting whether a skin treatment might cause brain damage and whether tattoo pigment would cross the BBB (blood brain border)-on-chip, e.g. at the time of tattooing, over-time, or during removal of a tattoo, might cause brain damage.

In some embodiments, a BBB-Chip/Brain-Chip may be used for direct testing of tattoo associated substances and compounds. In some embodiments, a BBB-Chip/Brain-Chip may be directly contacted with a pigment particle, a metal particle, an tattoo ink particle, etc., for: i) determining adverse effects upon brain associated endothelium, ii) determining whether particles can diffuse through or be transported through the BBB into the brain side or be taken up by brain cells contacting endothelial cells on the blood side and if so, iii) determining adverse effects upon cells whose cell bodies are located on the brain side. In some embodiments, a BBB-Chip/Brain-Chip may be treated with effluent from a treated Skin-Chip, as described herein, including a substance or compound treated Skin-Chip, a tattooed Skin-Chip, and a tattooed Skin-chip undergoing pigment removal, as described herein. In some embodiments, a BBB-Chip/Brain-Chip may be fluidically connected to a Skin-Chip, wherein the effluent of a Skin-Chip enters into and is flowed through a BBB-Chip/Brain-Chip.

FIG. 63 illustrates exemplary schematics of some embodiments of a BBB-chip/Brain-chip, as embodiments of a microfluidic device that may find use with the present invention.

F. Other Organs, One Example: Lung-Chip.

In some embodiments, a Lung-Chip, comprising Type I and Type II alveolar (epithelial) cells, may be used for direct testing of tattoo associated substances and compounds. In some embodiments, a Lung-Chip may be directly contacted with a pigment particle, a metal particle, an tattoo ink particle, etc., for determining adverse effects upon alveolar air sacs, for determining whether particles can diffuse into alveolar air sacs. In some embodiments, a Lung-Chip may be treated with effluent from a treated Skin-Chip, as described herein, including a substance or compound treated Skin-Chip, a tattooed Skin-Chip, and a tattooed Skin-chip undergoing pigment removal, as described herein. In some embodiments, a BBB-Chip/Brain-Chip may be fluidically connected to a Skin-Chip, wherein the effluent of a Skin-Chip enters into and is flowed through a Lung-Chip.

### SUMMARY

These studies provide a basis for better understanding the interaction with tattoo ink with skin on a cellular level, including: Mechanism of wound healing following tattoo injury; Pigment retention and distribution; Immune response

This model system is contemplated for use to develop better, safer products by:
Assess tattoo pigment safety in the skin
Assess systemic exposure and safety assessment of tattoo pigment on key functional organs
Develop safer—optimized formulations for Tattoo inks.
Work with regulatory agencies to set a new “science based standard” for the industry.

### DETAILED DESCRIPTION OF INVENTION

Cell Sources: The cells (e.g., parenchymal cells and/or vascular endothelial cells) used in the organ chips can be isolated from a tissue or a fluid of subject using any methods known in the art, or differentiated from stems cells, e.g., embryonic stem cells, or iPSC cells, or directly differentiated from somatic cells. In some embodiments, stem cells can be cultured inside the organ chips and be induced to differentiate to organ-specific cells. Alternatively, the cells used in the organ chips can be obtained from commercial sources, e.g., Cellular Dynamics International, Axiogenesis, Gigacyte, Biopredic, InVitrogen, Lonza, Clonetics, C D I, and Millipore, etc.).

In some embodiments, the cells used in the organ chips can be differentiated from the “established” cell lines that commonly exhibit poor differentiated properties (e.g., A549, CaCo2, HT29, etc.). These “established” cell lines can exhibit high levels of differentiation if presented with the relevant physical microenvironment (e.g., air-liquid interface and cyclic strain in lung, flow and cyclic strain in skin, lungs, etc.), e.g., in some embodiments of the organ chips.

In some embodiments, the cells used in the organ chips can be genetically engineered for various purposes, e.g., to express a fluorescent protein, or to modulate an expression of a gene, or to be sensitive to an external stimulus, e.g., light, pH, temperature and/or any combinations thereof.

Improvements in the open-top (OT) chip shall allow sufficient access to chamber for patterning gels. An improved OT Chip may be capable of applying stretch to gel within chamber. An improved OT chip may provide features to allow for gel attachment to the chamber and controlled shrinkage such that the region of interest remains viable for 4 weeks. An improved OT chip may allow for airflow over top channel and chamber. Allow for long term post experimental storage via standard organ-chip storage methods. Gel loading An improved OT chip may compatible with Chip Cradle 2.0 and/or ‘medium-sized Petri dish.

### I. Microfluidic Chips, Devices and Systems.

Microfluidic chips, devices, and systems contemplated for use include but are not limited to chips described in Bhatia and Ingber, “Microfluidic organs-on-chips.” Nature Biotechnology, 32(8):760-722, 2014; U.S. Pat. No. 8,647,861, Organ mimic device with microchannels and methods of use and manufacturing thereof, herein incorporated in its entirety, for some examples. The following section is merely for providing nonlimiting examples of embodiments that may find use as microfluidic devices.

Recreating the Cellular Microenvironment in Sink-Chips. Includes, Extracellular matrix and cell interactions; Cell shape and cytoarchitecture; Tissue-tissue interactions; Optional Mechanical forces; Dynamic system—Flow (except in the upper channel under ALI while flow continues in the lower channel 1); Resident or circulating immune cells

### II. Closed Top Chips.

In some embodiments, the present disclosure relates to a closed-top fluidic device, e.g. exemplary schematics in FIGS. 39A-C. The present disclosure relates to organ-on-chips, such as fluidic devices comprising one or more cells types for the simulation of one or more of the function of organ components. Accordingly, the present disclosure additionally describes closed-top liver-on-chips, kidney-on-chips, e.g. proximal tubule—kidney-on-chips, lung-on-chips, etc., see, e.g. schematic in FIG. 39C. The present disclosure also relates to lymph node-on-chips, and BBB (blood brain barrier)-on-chips, which may also use a fluidic device such as depicted schematically in FIGS. 39A-C.

The present disclosure additionally relates to fluidic devices comprising cells described herein as part of closed-top devices.

FIGS. 39A-B illustrates a perspective view of the devices in accordance with some embodiments described herein. For example, as shown in FIGS. 39A-1B, the device 200 can include a body 202 comprising a first structure 204 and a second structure 206 in accordance with an embodiment. The body 202 can be made of an elastomeric material, although the body can be alternatively made of a non-elastomeric material, or a combination of elastomeric and non-elastomeric materials. It should be noted that the microchannel design 203 is only exemplary and not limited to the configuration shown in FIGS. 39A-1B. While operating chambers 252 (e.g., as a pneumatics means to actuate the membrane 208, see the International Appl. No. PCT/US2009/050830 for further details of the operating chambers, the content of which is incorporated herein by reference in its entirety) are shown in FIGS. 39A-1B, they are not required in all of the embodiments described herein. In some embodiments, the devices do not comprise operating chambers on either side of the first chamber and the second chamber. In other embodiments, the devices described herein can be configured to provide other means to actuate the membrane, e.g., as described in the International Pat. Appl. No. PCT/US2014/071570, the content of which is incorporated herein by reference in its entirety.

In some embodiments, various organ chip devices described in the International Patent Application Nos. PCT/US2009/050830; PCT/US2012/026934; PCT/US2012/068725; PCT/US2012/068766; PCT/US2014/071611; and PCT/US2014/071570, the contents of each of which are incorporated herein by reference in their entireties, can be modified to form the devices described herein. For example, the organ chip devices described in those patent applications can be modified in accordance with the devices described herein.

The first structure 204 and/or second structure 206 can be fabricated from a rigid material, an elastomeric material, or a combination thereof. As used herein, the term “rigid” refers to a material that is stiff and does not bend easily, or maintains very close to its original form after pressure has been applied to it. The term “elastomeric” as used herein refers to a material or a composite material that is not rigid as defined herein. An elastomeric material is generally moldable and curable, and has an elastic property that enables the material to at least partially deform (e.g., stretching, expanding, contracting, retracting, compressing, twisting, and/or bending) when subjected to a mechanical force or pressure and partially or completely resume its original form or position in the absence of the mechanical force or pressure. In some embodiments, the term “elastomeric” can also refer to a material that is flexible/stretchable but does not resume its original form or position after pressure has been applied to it and removed thereafter. The terms “elastomeric” and “flexible” are interchangeably used herein.

In some embodiments, the material used to make the first structure and/or second structure or at least the portion of the first structure 204 and/or second structure 206 that is in contact with a gaseous and/or liquid fluid can comprise a biocompatible polymer or polymer blend, including but not limited to, polydimethylsiloxane (PDMS), polyurethane, polyimide, styrene-ethylene-butylene-styrene (SEBS), polypropylene, polycarbonate, cyclic polyolefin polymer/copolymer (COP/COC), or any combinations thereof. As used herein, the term “biocompatible” refers to any material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood.

Additionally or alternatively, at least a portion of the first structure 204 and/or second structure 206 can be made of non-flexible or rigid materials like glass, silicon, hard plastic, metal, or any combinations thereof.

The membrane 208 can be made of the same material as the first structure 204 and/or second structure 206 or a material that is different from the first structure 204 and/or second structure 206 of the devices described herein. In some embodiments, the membrane 208 can be made of a rigid material. In some embodiments, the membrane is a thermoplastic rigid material. Examples of rigid materials that can be used for fabrication of the membrane include, but are not limited to, polyester, polycarbonate or a combination thereof. In some embodiments, the membrane 208 can comprise a flexible material, e.g., but not limited to PDMS. Additional information about the membrane is further described below.

In some embodiments, the first structure and/or second structure of the device and/or the membrane can comprise or is composed of an extracellular matrix polymer, gel, and/or scaffold. Any extracellular matrix can be used herein, including, but not limited to, silk, chitosan, elastin, collagen, proteoglycans, hyaluronic acid, collagen, fibrin, and any combinations thereof.

The device in FIG. 39A can comprise a plurality of access ports 205. In addition, the branched configuration 203 can comprise a tissue-tissue interface simulation region (membrane 208 in FIG. 39B) where cell behavior and/or passage of gases, chemicals, molecules, particulates and cells are monitored.

FIG. 39B illustrates an exploded view of the device in accordance with an embodiment. In one embodiment, the body 202 of the device 200 comprises a first outer body portion (first structure) 204, a second outer body portion (second structure) 206 and an intermediary membrane 208 configured to be mounted between the first and second outer body portions 204, 206 when the portions 204, 206 are mounted to one another to form the overall body.

The first outer body portion or first structure 204 can have a thickness of any dimension, depending, in part, on the height of the first chamber 204. In some embodiments, the thickness of the first outer body portion or first structure 204 can be about 1 mm to about 100 mm, or about 2 mm to about 75 mm, or about 3 mm to about 50 mm, or about 3 mm to about 25 mm. In some embodiments, the first outer body portion or first structure 204 can have a thickness that is more than the height of the first chamber by no more than 5 mm, no more than 4 mm, no more than 3 mm, no more than 2 mm, no more than 1 mm, no more than 500 microns, no more than 400 microns, no more than 300 microns, no more than 200 microns, no more than 100 microns, no more than 70 microns or less. In some embodiments, it is desirable to keep the first outer body portion or first structure 204 as thin as possible such that cells on the membrane can be visualized or detected by microscopic, spectroscopic, and/or electrical sensing methods.

The second outer body portion or second structure 206 can have a thickness of any dimension, depending, in part, on the height of the second chamber 206. In some embodiments, the thickness of the second outer body portion or second structure 206 can be about 50 μm to about 10 mm, or about 75 μm to about 8 mm, or about 100 μm to about 5 mm, or about 200 μm to about 2.5 mm. In one embodiment, the thickness of the second outer body portion or second structure 206 can be about 1 mm to about 1.5 mm. In one embodiment, the thickness of the second outer body portion or second structure 206 can be about 0.2 mm to about 0.5 mm. In some embodiments, the second outer first structure and/or second structure portion 206 can have a thickness that is more than the height of the second chamber by no more than 5 mm, no more than 4 mm, no more than 3 mm, no more than 2 mm, no more than 1 mm, no more than 500 microns, no more than 400 microns, no more than 300 microns, no more than 200 microns, no more than 100 microns, no more than 70 microns or less. In some embodiments, it is desirable to keep the second outer body portion or second structure 206 as thin as possible such that cells on the membrane can be visualized or detected by microscopic, spectroscopic, and/or electrical sensing methods.

In some embodiments, the first chamber and the second chamber can each independently comprise a channel. The channel(s) can be substantially linear or they can be non-linear. In some embodiments, the channels are not limited to straight or linear channels and can comprise curved, angled, or otherwise non-linear channels. It is to be further understood that a first portion of a channel can be straight, and a second portion of the same channel can be curved, angled, or otherwise non-linear. Without wishing to be bound by a theory, a non-linear channel can increase the ratio of culture area to device area, thereby providing a larger surface area for cells to grow. This can also allow for a higher amount or density of cells in the channel.

FIG. 39B illustrates an exploded view of the device in accordance with an embodiment. As shown in FIG. 39B, the first outer body portion 204 includes one or more inlet fluid ports 210 preferably in communication with one or more corresponding inlet apertures 211 located on an outer surface of the body 202. The device 100 is preferably connected to the fluid source 104 via the inlet aperture 211 in which fluid travels from the fluid source 104 into the device 100 through the inlet fluid port 210.

Additionally, the first outer body portion or first structure 204 can include one or more outlet fluid ports 212 in communication with one or more corresponding outlet apertures 215 on the outer surface of the first structure 204. In some embodiments, a fluid passing through the device 200 can exit the device to a fluid collector or other appropriate component via the corresponding outlet aperture 215. It should be noted that the device 200 can be set up such that the fluid port 210 is an outlet and fluid port 212 is an inlet.

In some embodiments, as shown in FIG. 39B, the device 200 can comprise an inlet channel 225 connecting an inlet fluid port 210 to the first chamber 204. The inlet channels and inlet ports can be used to introduce cells, agents (e.g., but not limited to, stimulants, drug candidate, particulates), airflow, and/or cell culture media into the first chamber 204.

The device 200 can also comprise an outlet channel 227 connecting an outlet fluid port 212 to the first chamber 204. The outlet channels and outlet ports can also be used to introduce cells, agents (e.g., but not limited to, stimulants, drug candidate, particulates), airflow, and/or cell culture media into the first chamber 204.

Although the inlet and outlet apertures 211, 215 are shown on the top surface of the first structure 204 and are located perpendicular to the inlet and outlet channels 225, 227, one or more of the apertures 211, 215 can be located on one or more lateral surfaces of the first structure and/or second structure such that at least one of the inlet and outlet apertures 211, 215 can be in-plane with the inlet and/or outlet channels 225, 227, respectively, and/or be oriented at an angle from the plane of the inlet and/or outlet channels 225, 227.

In another embodiment, the fluid passing between the inlet and outlet fluid ports can be shared between the first chamber 204 and second chamber 206. In either embodiment, characteristics of the fluid flow, such as flow rate, fluid type and/or composition, and the like, passing through the first chamber 204 can be controllable independently of fluid flow characteristics through the second chamber 206 and vice versa.

In some embodiments, while not necessary, the first structure 204 can include one or more pressure inlet ports 214 and one or more pressure outlet ports 216 in which the inlet ports 214 are in communication with corresponding apertures 217 located on the outer surface of the device 200. Although the inlet and outlet apertures are shown on the top surface of the first structure 204, one or more of the apertures can alternatively be located on one or more lateral sides of the first structure and/or second structure. In operation, one or more pressure tubes (not shown) connected to an external force source (e.g., pressure source) can provide positive or negative pressure to the device via the apertures 217. Additionally, pressure tubes (not shown) can be connected to the device 200 to remove the pressurized fluid from the outlet port 216 via the apertures 223. It should be noted that the device 200 can be set up such that the pressure port 214 is an outlet and pressure port 216 is an inlet. It should be noted that although the pressure apertures 217, 223 are shown on the top surface of the first structure 204, one or more of the pressure apertures 217, 223 can be located on one or more side surfaces of the first structure 204.

Referring to FIG. 39B, in some embodiments, the second structure 206 can include one or more inlet fluid ports 218 and one or more outlet fluid ports 220. As shown in FIG. 39B, the inlet fluid port 218 is in communication with aperture 219 and outlet fluid port 220 is in communication with aperture 221, whereby the apertures 219 and 221 are located on the outer surface of the second structure 206. Although the inlet and outlet apertures are shown on the surface of the second structure, one or more of the apertures can be alternatively located on one or more lateral sides of the second structure.

As with the first outer body portion or first structure 204 described above, one or more fluid tubes connected to a fluid source can be coupled to the aperture 219 to provide fluid to the device 200 via port 218. Additionally, fluid can exit the device 200 via the outlet port 220 and outlet aperture 221 to a fluid reservoir/collector or other component. It should be noted that the device 200 can be set up such that the fluid port 218 is an outlet and fluid port 220 is an inlet.

In some embodiments, the second outer body portion and/or second structure 206 can include one or more pressure inlet ports 222 and one or more pressure outlet ports 224. In some embodiments, the pressure inlet ports 222 can be in communication with apertures 227 and pressure outlet ports 224 are in communication with apertures 229, whereby apertures 227 and 229 are located on the outer surface of the second structure 206. Although the inlet and outlet apertures are shown on the bottom surface of the second structure 206, one or more of the apertures can be alternatively located on one or more lateral sides of the second structure. Pressure tubes connected to an external force source (e.g., pressure source) can be engaged with ports 222 and 224 via corresponding apertures 227 and 229. It should be noted that the device 200 can be set up such that the pressure port 222 is an outlet and fluid port 224 is an inlet.

In some embodiments where the operating channels (e.g., 252 shown in FIG. 39A) are not mandatory, the first structure 204 does not require any pressure inlet port 214, pressure outlet port 216. Similarly, the second structure 206 does not require any pressure inlet port 222 or pressure outlet port 224.

In an embodiment, the membrane 208 is mounted between the first structure 204 and the second structure 206, whereby the membrane 208 is located within the first structure 204 and/or second structure 206 of the device 200. In an embodiment, the membrane 208 is a made of a material having a plurality of pores or apertures therethrough, whereby molecules, cells, fluid or any media is capable of passing through the membrane 208 via one or more pores in the membrane 208. As discussed in more detail below, the membrane 208 in one embodiment can be made of a material which allows the membrane 208 to undergo stress and/or strain in response to an external force (e.g., cyclic stretching or pressure). In one embodiment, the membrane 208 can be made of a material, which allows the membrane 208 to undergo stress and/or strain in response to pressure differentials present between the first chamber 204, the second chamber 206 and the operating channels 252. Alternatively, the membrane 208 is relatively inelastic or rigid in which the membrane 208 undergoes minimal or no movement.

In some embodiments where the device simulates a function of a tissue, such as a lymph node, the membrane can be rigid.

The first chamber 204 and/or the second chamber 206 can have a length suited to the need of an application (e.g., a physiological system to be modeled), desirable size of the device, and/or desirable size of the view of field. In some embodiments, the first chamber 204 and/or the second chamber 206 can have a length of about 0.5 cm to about 10 cm. In one embodiment, the first chamber 204 and/or the second chamber 206 can have a length of about 1 cm to about 3 cm. In one embodiment, the first chamber 204 and/or the second chamber 206 can have a length of about 2 cm.

The width of the first chamber and/or the second chamber can vary with desired cell growth surface area. The first chamber 204 and the second chamber 206 can each have a range of width dimension between 100 microns and 50 mm, or between 200 microns and 10 mm, or between 200 microns and 1500 microns, or between 400 microns and 1 mm, or between 50 microns and 2 mm, or between 100 microns and 5 mm. In some embodiments, the first chamber 204 and the second chamber 206 can each have a width of about 500 microns to about 2 mm. In some embodiments, the first chamber 204 and the second chamber 206 can each have a width of about 1 mm.

In some embodiments, the widths of the first chamber and the second chamber can be configured to be different, with the centers of the chambers aligned or not aligned. In some embodiments, the channel heights, widths, and/or cross sections can vary along the length of the devices described herein.

The heights of the first chamber and the second chamber can vary to suit the needs of desired applications (e.g., to provide a low shear stress, and/or to accommodate cell size). The first chamber and the second chamber of the devices described herein can have the same heights or different heights. In some embodiments, the height of the second chamber 206 can be substantially the same as the height of the first chamber 204.

In some embodiments, the height of at least a length portion of the first chamber 204 (e.g., the length portion where the cells are designated to grow) can be substantially greater than the height of the second chamber 206 within the same length portion. For example, the height ratio of the first chamber to the second chamber can be greater than 1:1, including, for example, greater than 1.1:1, 1.5:1, 2:1, 2.5:1, 3:1, 3.5:1, 4:1, 4.5:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1. In some embodiments, the height ratio of the first chamber to the second chamber can range from 1.1:1 to about 50:1, or from about 2.5:1 to about 50:1, or from 2.5 to about 25:1, or from about 2.5:1 to about 15:1. In one embodiment, the height ratio of the first chamber to the second chamber ranges from about 1:1 to about 20:1. In one embodiment, the height ratio of the first chamber to the second chamber ranges from about 1:1 to about 15:1. In one embodiment, the height ratio of the first chamber to the second chamber can be about 10:1.

The height of the first chamber 204 can be of any dimension, e.g., sufficient to accommodate cell height and/or to permit a low shear flow. For example, in some embodiments, the height of the first chamber can range from about 100 μm to about 50 mm, about 200 μm to about 10 mm, about 500 μm to about 5 mm, or about 750 um to about 2 mm. In one embodiment, the height of the first chamber can be about 150 um. In one embodiment, the height of the first chamber can be about 1 mm.

The height of the second chamber 206 can be of any dimension provided that the flow rate and/or shear stress of a medium flowing in the second chamber can be maintained within a physiological range, or does not cause any adverse effect to the cells. In some embodiments, the height of the second chamber can range from 20 μm to about 1 mm, or about 50 μm to about 500 μm, or about 75 μm to about 400 μm, or about 100 μm to about 300 μm. In one embodiment, the height of the second chamber can be about 150 μm. In one embodiment, the height of the second chamber can be about 100 μm.

The first chamber and/or the second chamber can have a uniform height along the length of the first chamber and/or the second chamber, respectively. Alternatively, the first chamber and/or the second chamber can each independently have a varying height along the length of the first chamber and/or the second chamber, respectively. For example, a length portion of the first chamber can be substantially taller than the same length portion of the second chamber, while the rest of the first chamber can have a height comparable to or even smaller than the height of the second chamber.

In some embodiments, the first structure and/or second structure of the devices described herein can be further adapted to provide mechanical modulation of the membrane. Mechanical modulation of the membrane can include any movement of the membrane that is parallel to and/or perpendicular to the force/pressure applied to the membrane, including, but are not limited to, stretching, bending, compressing, vibrating, contracting, waving, or any combinations thereof. Different designs and/or approaches to provide mechanical modulation of the membrane between two chambers have been described, e.g., in the International Patent App. Nos. PCT/US2009/050830, and PCT/US2014/071570, the contents of which are incorporated herein by reference in their entireties, and can be adapted herein to modulate the membrane in the devices described herein.

In some embodiments, the devices described herein can be placed in or secured to a cartridge. In accordance with some embodiments of some aspects described herein, the device can be integrated into a cartridge and form a monolithic part. Some examples of a cartridge are described in the International Patent App. No. PCT/US2014/047694, the content of which is incorporated herein by reference in its entirety. The cartridge can be placed into and removed from a cartridge holder that can establish fluidic connections upon or after placement and optionally seal the fluidic connections upon removal. In some embodiments, the cartridge can be incorporated or integrated with at least one sensor, which can be placed in direct or indirect contact with a fluid flowing through a specific portion of the cartridge during operation. In some embodiments, the cartridge can be incorporated or integrated with at least one electric or electronic circuit, for example, in the form of a printed circuit board or flexible circuit. In accordance with some embodiments of some aspects described herein, the cartridge can comprise a gasketing embossment to provide fluidic routing.

In some embodiments, the cartridge and/or the device described herein can comprise a barcode. The barcode can be unique to types and/or status of the cells present on the membrane. Thus, the barcode can be used as an identifier of each device adapted to mimic function of at least a portion of a specific tissue and/or a specific tissue-specific condition. Prior to operation, the barcode of the cartridge can be read by an instrument so that the cartridge can be placed and/or aligned in a cartridge holder for proper fluidic connections and/or proper association of the data obtained during operation of each device. In some embodiments, data obtained from each device include, but are not limited to, cell response, immune cell recruitment, intracellular protein expression, gene expression, cytokine/chemokine expression, cell morphology, functional data such as effectiveness of an endothelium as a barrier, concentration change of an agent that is introduced into the device, or any combinations thereof.

In some embodiments, the device can be connected to the cartridge by an interconnect adapter that connects some or all of the inlet and outlet ports of the device to microfluidic channels or ports on the cartridge. Some examples interconnect adapters are disclosed in U.S. Provisional Application No. 61/839,702, filed on Jun. 26, 2013, and the International Patent Application No. PCT/US2014/044417 filed Jun. 26, 2014, the contents of each of which are hereby incorporated by reference in their entirety. The interconnect adapter can include one or more nozzles having fluidic channels that can be received by ports of the device described herein. The interconnect adapter can also include nozzles having fluidic channels that can be received by ports of the cartridge.

In some embodiments, the interconnect adaptor can comprise a septum interconnector that can permit the ports of the device to establish transient fluidic connection during operation, and provide a sealing of the fluidic connections when not in use, thus minimizing contamination of the cells and the device. Some examples of a septum interconnector are described in U.S. Provisional Application No. 61/810,944 filed Apr. 11, 2013, the content of which is incorporated herein by reference in its entirety.

Membrane: The membrane 208 is oriented along a plane 208P parallel to the x-y plane between the first chamber 204 and the second chamber 206. It should be noted that although one membrane 208, more than one membrane 208 can be configured in devices which comprise more than two chambers. FIG. 39A and FIG. 39B.

FIG. 39A-C illustrates embodiments of an exemplary S1 microfluidic device which may find use with the present invention.

FIG. 39A Illustrates a perspective view of a microfluidic device with microfluidic channels in accordance with an embodiment.

FIG. 39B Illustrates an exploded view of the device 200 in accordance with an embodiment, showing a microfluidic channel in a top piece 207 and a microfluidic channel in a bottom piece, separated by a membrane 208.

FIG. 39C shows cells in relation to device parts in a closed top chip, e.g. upper microchannel (1-blue); lower microchannel (2-red) and optional vacuum chamber (6). 1. Options include a liquid microchannel; air-liquid microchannel (upper); 2. Vascular channel (lower); 3. parenchymal cells, including but not limited to epithelial cells/tissue (e.g. liver, kidney, lung), other types of cells, reticular cells (e.g. lymph node), neuronal cells, pericytes astrocytes (e.g. brain); 4. Simulated capillaries (e.g. endothelial cells matching or compatible with the cells in the upper chamber); 5. Membrane, stretchable; and 6. Vacuum Channels. Arrows represent direction of fluid flow.

The membrane separating the first chamber and the second chamber in the devices described herein can be porous (e.g., permeable or selectively permeable), non-porous (e.g., non-permeable), rigid, flexible, elastic or any combinations thereof. Accordingly, the membrane 208 can have a porosity of about 0% to about 99%. As used herein, the term “porosity” is a measure of total void space (e.g., through-holes, openings, interstitial spaces, and/or hollow conduits) in a material, and is a fraction of volume of total voids over the total volume, as a percentage between 0 and 100% (or between 0 and 1). A membrane with substantially zero porosity is non-porous or non-permeable.

As used interchangeably herein, the terms “non-porous” and “non-permeable” refer to a material that does not allow any molecule or substance to pass through.

In some embodiments, the membrane can be porous and thus allow molecules, cells, particulates, chemicals and/or media to migrate or transfer between the first chamber 204 and the second chamber 206 via the membrane 208 from the first chamber 204 to the second chamber 206 or vice versa.

As used herein, the term “porous” generally refers to a material that is permeable or selectively permeable. The term “permeable” as used herein means a material that permits passage of a fluid (e.g., liquid or gas), a molecule, a whole living cell and/or at least a portion of a whole living cell, e.g., for formation of cell-cell contacts. The term “selectively permeable” as used herein refers to a material that permits passage of one or more target group or species, but act as a barrier to non-target groups or species. For example, a selectively-permeable membrane can allow passage of a fluid (e.g., liquid and/or gas), nutrients, wastes, cytokines, and/or chemokines from one side of the membrane to another side of the membrane, but does not allow whole living cells to pass therethrough. In some embodiments, a selectively-permeable membrane can allow certain cell types to pass therethrough but not other cell types.

he permeability of the membrane to individual matter/species can be determined based on a number of factors, including, e.g., material property of the membrane (e.g., pore size, and/or porosity), interaction and/or affinity between the membrane material and individual species/matter, individual species size, concentration gradient of individual species between both sides of the membrane, elasticity of individual species, and/or any combinations thereof.

A porous membrane can have through-holes or pore apertures extending vertically and/or laterally between two surfaces 208A and 208B of the membrane (FIG. 39B), and/or a connected network of pores or void spaces (which can, for example, be openings, interstitial spaces or hollow conduits) throughout its volume. The porous nature of the membrane can be contributed by an inherent physical property of the selected membrane material, and/or introduction of conduits, apertures and/or holes into the membrane material.

In some embodiments, a membrane can be a porous scaffold or a mesh. In some embodiments, the porous scaffold or mesh can be made from at least one extracellular matrix polymer (e.g., but not limited to collagen, alginate, gelatin, fibrin, laminin, hydroxyapatite, hyaluronic acid, fibroin, and/or chitosan), and/or a biopolymer or biocompatible material (e.g., but not limited to, polydimethylsiloxane (PDMS), polyurethane, styrene-ethylene-butylene-styrene (SEBS), poly(hydroxyethylmethacrylate) (pHEMA), polyethylene glycol, polyvinyl alcohol and/or any biocompatible material described herein for fabrication of the device first structure and/or second structure) by any methods known in the art, including, e.g., but not limited to, electrospinning, cryogelation, evaporative casting, and/or 3D printing. See, e.g., Sun et al. (2012) “Direct-Write Assembly of 3D Silk/Hydroxyapatite Scaffolds for Bone Co-Cultures.” Advanced Healthcare Materials, no. 1: 729-735; Shepherd et al. (2011) “3D Microperiodic Hydrogel Scaffolds for Robust Neuronal Cultures.” Advanced Functional Materials 21: 47-54; and Barry III et al. (2009) “Direct-Write Assembly of 3D Hydrogel Scaffolds for Guided Cell Growth.” Advanced Materials 21: 1-4, for examples of a 3D biopolymer scaffold or mesh that can be used as a membrane in the device described herein.

In some embodiments, a membrane can comprise an elastomeric portion fabricated from a styrenic block copolymer-comprising composition, e.g., as described in the International Pat. App. No. PCT/US2014/071611, can be adopted in the devices described herein, the contents of each of which are incorporated herein by reference in its entirety. In some embodiments, the styrenic block copolymer-comprising composition can comprise SEBS and polypropylene.

In some embodiments, a membrane can be a hydrogel or a gel comprising an extracellular matrix polymer, and/or a biopolymer or biocompatible material. In some embodiments, the hydrogel or gel can be embedded with a conduit network, e.g., to promote fluid and/or molecule transport. See, e.g., Wu et al. (2011) “Omnidirectional Printing of 3D Microvascular Networks.” Advanced Materials 23: H178-H183; and Wu et al. (2010) “Direct-write assembly of biomimetic microvascular networks for efficient fluid transport.” Soft Matter 6: 739-742, for example methods of introducing a conduit network into a gel material.

In some embodiments, a porous membrane can be a solid biocompatible material or polymer that is inherently permeable to at least one matter/species (e.g., gas molecules) and/or permits formation of cell-cell contacts. In some embodiments, through-holes or apertures can be introduced into the solid biocompatible material or polymer, e.g., to enhance fluid/molecule transport and/or cell migration. In one embodiment, through-holes or apertures can be cut or etched through the solid biocompatible material such that the through-holes or apertures extend vertically and/or laterally between the two surfaces of the membrane 208A and 208B. It should also be noted that the pores can additionally or alternatively incorporate slits or other shaped apertures along at least a portion of the membrane 208 which allow cells, particulates, chemicals and/or fluids to pass through the membrane 208 from one section of the central channel to the other.

The pores of the membrane (including pore apertures extending through the membrane 208 from the top 208A to bottom 208B surfaces thereof and/or a connected network of void space within the membrane 208) can have a cross-section of any size and/or shape. For example, the pores can have a pentagonal, circular, hexagonal, square, elliptical, oval, diamond, and/or triangular shape.

The cross-section of the pores can have any width dimension provided that they permit desired molecules and/or cells to pass through the membrane. In some embodiments, the pore size of the membrane should be big enough to provide the cells sufficient access to nutrients present in a fluid medium flowing through the first chamber and/or the second chamber. In some embodiments, the pore size can be selected to permit passage of cells (e.g., immune cells) from one side of the membrane to the other. In some embodiments, the pore size can be selected to permit passage of nutrient molecules. In some embodiments, the width dimension of the pores can be selected to permit molecules, particulates and/or fluids to pass through the membrane 208 but prevent cells from passing through the membrane 208. In some embodiments, the width dimension of the pores can be selected to permit cells, molecules, particulates and/or fluids to pass through the membrane 208. Thus, the width dimension of the pores can be selected, in part, based on the sizes of the cells, molecules, and/or particulates of interest. In some embodiments, the width dimension of the pores (e.g., diameter of circular pores) can be in the range of 0.01 microns and 20 microns, or in one embodiment, approximately 0.1-15 microns, or approximately 1-10 microns. In one embodiment, the pores have a width of about 7 microns.

In an embodiment, the porous membrane 208 can be designed or surface patterned to include micro and/or nanoscopic patterns therein such as grooves and ridges, whereby any parameter or characteristic of the patterns can be designed to desired sizes, shapes, thicknesses, filling materials, and the like.

The membrane 208 can have any thickness to suit the needs of a target application. In some embodiments, the membrane can be configured to deform in a manner (e.g., stretching, retracting, compressing, twisting and/or waving) that simulates a physiological strain experienced by the cells in its native microenvironment. In these embodiments, a thinner membrane can provide more flexibility. In some embodiments, the membrane can be configured to provide a supporting structure to permit growth of a defined layer of cells thereon. Thus, in some embodiments, a thicker membrane can provide a greater mechanical support. In some embodiments, the thickness of the membrane 208 can range between 70 nanometers and 100 μm, or between 1 μm and 100 μm, or between 10 and 100 μm. In one embodiment, the thickness of the membrane 208 can range between 10 μm and 80 μm. In one embodiment, the thickness of the membrane 208 can range between 30 μm and 80 μm. In one embodiment, the thickness of the membrane 208 can be about 50 μm.

While the membrane 208 generally have a uniform thickness across the entire length or width, in some embodiments, the membrane 208 can be designed to include regions which have lesser or greater thicknesses than other regions in the membrane 208. The decreased thickness area(s) can run along the entire length or width of the membrane 208 or can alternatively be located at only certain locations of the membrane 208. The decreased thickness area can be present along the bottom surface of the membrane 208 (i.e. facing second chamber 206), or additionally/alternatively be on the opposing surface of the membrane 208 (i.e. facing second chamber 204). It should also be noted that at least portions of the membrane 208 can have one or more larger thickness areas relative to the rest of the membrane, and capable of having the same alternatives as the decreased thickness areas described above.

In some embodiments, the membrane can be coated with substances such as various cell adhesion promoting substances or ECM proteins, such as fibronectin, laminin, various collagen types, glycoproteins, vitronectin, elastins, fibrin, proteoglycans, heparin sulfate, chondroitin sulfate, keratin sulfate, hyaluronic acid, fibroin, chitosan, or any combinations thereof. In some embodiments, one or more cell adhesion molecules can be coated on one surface of the membrane 208 whereas another cell adhesion molecule can be applied to the opposing surface of the membrane 208, or both surfaces can be coated with the same cell adhesion molecules. In some embodiments, the ECMs, which can be ECMs produced by cells, such as primary cells or embryonic stem cells, and other compositions of matter are produced in a serum-free environment.

In an embodiment, one can coat the membrane with a cell adhesion factor and/or a positively-charged molecule that are bound to the membrane to improve cell attachment and stabilize cell growth. The positively charged molecule can be selected from the group consisting of polylysine, chitosan, poly(ethyleneimine) or acrylics polymerized from acrylamide or methacrylamide and incorporating positively-charged groups in the form of primary, secondary or tertiary amines, or quaternary salts. The cell adhesion factor can be added to the membrane and is fibronectin, laminin, various collagen types, glycoproteins, vitronectin, elastins, fibrin, proteoglycans, heparin sulfate, chondroitin sulfate, keratin sulfate, hyaluronic acid, tenascin, antibodies, aptamers, or fragments or analogs having a cell binding domain thereof. The positively-charged molecule and/or the cell adhesion factor can be covalently bound to the membrane. In another embodiment, the positively-charged molecule and/or the cell adhesion factor are covalently bound to one another and either the positively-charged molecule or the cell adhesion factor is covalently bound to the membrane. Also, the positively-charged molecule or the cell adhesion factor or both can be provided in the form of a stable coating non-covalently bound to the membrane.

In an embodiment, the cell attachment-promoting substances, matrix-forming formulations, and other compositions of matter are sterilized to prevent unwanted contamination. Sterilization can be accomplished, for example, by ultraviolet light, filtration, gas plasma, ozone, ethylene oxide, and/or heat. Antibiotics can also be added, particularly during incubation, to prevent the growth of bacteria, fungi and other undesired micro-organisms. Such antibiotics include, by way of non-limiting example, gentamicin, streptomycin, penicillin, amphotericin and ciprofloxacin.

In some embodiments, the membrane and/or other components of the devices described herein can be treated using gas plasma, charged particles, ultraviolet light, ozone, or any combinations thereof.

Using the devices described herein, one can study biotransformation, absorption, as well as drug clearance, metabolism, delivery, and toxicity. The activation of xenobiotics can also be studied. The bioavailability and transport of chemical and biological agents across epithelial layers as in a tissue or organ, e.g., lung, and across endothelial layers as in blood vessels, such as for a BBB-on-chip, and across embodiments of skin epithelial layers for drug metabolism can also be studied. The acute basal toxicity, acute local toxicity or acute organ-specific toxicity, teratogenicity, genotoxicity, carcinogenicity, and mutagenicity, of chemical agents can also be studied. Effects of infectious biological agents, biological weapons, harmful chemical agents and chemical weapons can also be detected and studied. Infectious diseases and the efficacy of chemical and biological agents to treat these diseases, as well as optimal dosage ranges for these agents, can be studied. The response of organs in vivo to chemical and biological agents, and the pharmacokinetics and pharmacodynamics of these agents can be detected and studied using the devices described herein. The impact of genetic content on response to the agents can be studied. The amount of protein and gene expression in response to chemical or biological agents can be determined. Changes in metabolism in response to chemical or biological agents can be studied as well using devices described herein.

In some embodiments, the devices described herein (e.g., a Skin-on-Chip) can be used to assess the clearance of a test compound. For clearance studies, the disappearance of a test compound can be measured (e.g. using mass spec) in the media of the top chamber, bottom chamber, or both chambers (divided by a membrane comprising intestinal epithelial cells).

For example, in accordance to one aspect of the invention, a Skin-on-Chip drug-metabolizing performance can be measured by i) disposing a substrate compound with known liver metabolites in the media of the top chamber, bottom chamber, or both chambers; and ii) measuring the amount of generated metabolite in the media of the top chamber, bottom chamber or both chambers (e.g. using mass spec). As is known in the art, the choice of the substrate and measured metabolite can help provide information on specific liver drug-metabolism enzymes (e.g. CYP450 isoforms, Phase II enzymes, etc.)

In some embodiments, the devices described herein (e.g., a Skin-on-Chip) can be used to assess the induction or inhibition potential of a test compound. For induction or inhibition studies a variety of tests are contemplated. For example, induction of CYP3A4 activity in the liver is one of main causes of drug-drug interactions, which is a mechanism to defend against exposure to drugs and toxin, but can also lead to unwanted side-effects (toxicity) or change the efficacy of a drug. A reliable and practical CYP3A induction assay with human hepatocytes in a 96-well format has been reported, where various 96-well plates with different basement membrane were evaluated using prototypical inducers, rifampicin, phenytoin, and carbamazepine. See Drug Metab. Dispo. (2010) November; 38(11):1912-6.

According to one aspect of the invention, the induction or inhibition potential of a test compound at a test concentration can be evaluated by i) disposing the test compound in the media of the top chamber, bottom chamber or both chambers at the test concentration; ii) exposing the device for a selected period of time; and iii) assessing the induction or inhibition of enzymes by comparing performance to a measurement performed before the test compound was applied, to a measurement performed on a Skin-on-Chip that was subjected to a lower concentration of test compound (or no test compound at all), or both. In some embodiments, the performance measurement can comprise an RNA expression level. In some embodiments, the performance measurement comprises assessing drug-metabolizing capacity.

In some embodiments, the devices described herein (e.g., a Skin-on-Chip) can be used to identify in vivo metabolites of a test compound or agent, and optionally the in vivo ratio of these metabolites. According to one aspect of the invention, in vivo metabolites can be identified by i) disposing a test compound or agent in the media of the top chamber, bottom chamber, or both chambers; and ii) measuring the concentration of metabolites in the media of the top chamber, bottom chamber, or both chambers. In some embodiments, the measuring of the concentration of metabolites comprises mass spectroscopy.

In some embodiments, the devices described herein (e.g., a Skin-on-Chip) can be used to identify the toxicity of a test compound or agent at a test concentration. According to one aspect of the invention, toxicity can be evaluated by i) disposing a test compound in the media of the top chamber, bottom chamber, or both chambers; and ii) measuring one or more toxicity endpoints selected from the list of leakage of cellular enzymes (e.g., lactose dehydrogenase, alanine aminotransferase, aspartate aminotransferase) or material (e.g., adenosine triphosphate), variation in RNA expression, inhibition of drug-metabolism capacity, reduction of intracellular ATP (adenosine triphosphate), cell death, apoptosis, and cell membrane degradation.

A. Closed Top Microfluidic Chips without Gels.

In one embodiment, closed top organ-on-chips do not contain gels, either as a bulk gel or a gel layer. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer.

Additional embodiments are described herein that may be incorporated into closed top chips without gels.

B. Closed Top Microfluidic Chips with Gels.

In one embodiment, closed top organ-on-chips do contain gels, such as a gel layer, or bulk gel, including but not limited to a gel matrix, hydrogel, etc. Thus, in one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. In some embodiments, the device further comprises a gel. In some embodiments, the gel is a continuous layer. In some embodiments, the gel is a layer of approximately the same thickness across the layer. In some embodiments, the gel is a discontinuous layer. In some embodiments, the gel has different thicknesses across the layer. In some embodiments, the first side of the membrane may have a gel layer. In some embodiments, a gel is added to the first side of the membrane without an ECM layer. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer. In some embodiments, the gel layer is above the ECM coating layer. In some embodiments, the ECM coating layer may have a gel layer on the bottom, i.e. the side facing the membrane. In some embodiments, the gel overlays the ECM gel layer.

Additional embodiments are described herein that may be incorporated into closed top chips with gels.

C. Closed Top Microfluidic Chips with Simulated Lumens.

A closed top organ-on-chip comprising a gel-lined simulated lumen may be used for generating a more physiological relevant model of gastrointestinal tissue. In some embodiments, closed top organ-on-chips further comprise a gel simulated three-dimensional (3-D) lumen. In other words, a 3-D lumen may be formed using gels by providing simulated intestinal villi (e.g. viscous fingers) and/or mimicking intestinal folds. In a preferred embodiment, the gel forms a lumen, i.e. by viscous fingering patterning.

Using viscous fingering techniques, e.g. viscous fingering patterning, a simulated intestinal lumen may be formed by numerous simulated intestinal villi structures. Intestinal villi (singular: villus) refer to small, finger-like projections that extend into the lumen of the small intestine. For example, healthy small intestine mucosa contains these small finger-like projections of tissue that are present along the lumen as folds of circular plica finger-like structures. A villus is lined on the luminal side by an epithelia cell layer, where the microvillus of the epithelial cells (enterocytes) faces the lumen (i.e. apical side). Viscous fingers may be long and broad, for mimicking villi in the duodenum of the small intestine, while thinner or shorter viscous fingers may be used for mimicking villi in other parts of the gastrointestinal tract. As one example, viscous fingers may be formed and used to mimic epithelial projections in the colon.

Methods to create three-dimensional (3-D) lumen structures in permeable matrices are known in the art. One example of a 3-D structure forming at least one lumen is referred to as “viscous fingering”. One example of viscous fingering methods that may be used to for form lumens, e.g. patterning lumens, is described by Bischel, et al. “A Practical Method for Patterning Lumens through ECM Hydrogels via Viscous Finger Patterning.” J Lab Autom. 2012 April; 17(2): 96-103. Author manuscript; available in PMC 2012 Jul. 16, herein incorporated by reference in its entirety. In one example of a viscous finger patterning method for use with microfluidic organ-on-chips, lumen structures are patterned with an ECM hydrogel.

“Viscous” generally refers to a substance in between a liquid and a solid, i.e. having a thick consistency. A “viscosity” of a fluid refers to a measure of its resistance to gradual deformation by shear stress or tensile stress. For liquids, it corresponds to an informal concept of “thickness”; for example, honey has a much higher viscosity than water.

“Viscous fingering” refers in general to the formation of patterns in “a morphologically unstable interface between two fluids in a porous medium.

A “viscous finger” generally refers to the extension of one fluid into another fluid. Merely as an example, a flowable gel or partially solidified gel may be forced, by viscous fingering techniques, into another fluid, into another viscous fluid in order to form a viscous finger, i.e. simulated intestinal villus.

In some embodiments, the lumen can be formed by a process comprising (i) providing the first chamber filled with a viscous solution of the first matrix molecules; (ii) flowing at least one or more pressure-driven fluid(s) with low viscosity through the viscous solution to create one or more lumens each extending through the viscous solution; and (iii) gelling, polymerizing, and/or cross linking the viscous solution. Thus, one or a plurality of lumens each extending through the first permeable matrix can be created.

In another embodiment, gel is added to a channel for making a lumen.

In some embodiments as described herein, the first and second permeable matrices can each independently comprise a hydrogel, an extracellular matrix gel, a polymer matrix, a monomer gel that can polymerize, a peptide gel, or a combination of two or more thereof. In one embodiment, the first permeable matrix can comprise an extracellular matrix gel, (e.g. collagen). In one embodiment, the second permeable matrix can comprise an extracellular matrix gel and/or protein mixture gel representing an extracellular miroenvironment, (e.g. MATRIGEL®. In some embodiments, the first and second permeable matrixes can each independently comprise a polymer matrix. Methods to create a permeable polymer matrix are known in the art, including, e.g. but not limited to, particle leaching from suspensions in a polymer solution, solvent evaporation from a polymer solution, sold-liquid phase separation, liquid—liquid phase separation, etching of specific “block domains” in block co-polymers, phase separation to block-co-polymers, chemically cross-linked polymer networks with defined permabilities, and a combination of two or more thereof.

Another example for making branched structures using fluids with differing viscosities is described in “Method And System For Integrating Branched Structures In Materials” to Katrycz, Publication number US20160243738, herein incorporated by reference in its entirety.

Regardless of the type of lumen formed by a gel and/or structure, cells can be attached to theses structures either to lumen side of the gel and/or within the gel and/or on the side of the gel opposite the lumen. Thus, three-dimensional (3-D) lumen gel structures may be used in several types of embodiments for closed top microfluidic chips, e.g. epithelial cells can be attached to outside of the gel, or within the gel. In some embodiments, LPDCs may be added within the gel, or below the gel, on the opposite side of the lumen. In some embodiments, stoma cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

Additional embodiments are described herein that may be incorporated into closed top chips with simulated 3D lumens containing a gel.

D. Double Membrane Devices (Chips).

In one embodiment, a chip having at least two membranes and at least 3 channels is used for providing one embodiment of a lymph node-chip, for one example. In some embodiments, a chip described in U.S. Pat. No. 8,647,861, herein incorporated in its entirety, is used in at least one step for providing innervated brain-on-chip (including a BBB-chip). In one embodiment, a chip having at least two membranes and at least 3 channels is used for providing neuronal cells.

FIG. 50A illustrates a perspective view of an organ mimic device in accordance with an embodiment that contains three parallel microchannels separated by two porous membranes. As shown in FIG. 50A, the organ mimic device 800 includes operating microchannels 802 and an overall central microchannel 804 positioned between the operating microchannels 802. The overall central microchannel 804 includes multiple membranes 806A, 806B positioned along respective parallel x-y planes which separate the microchannel 804 into three distinct central microchannels 804A, 804B and 804C. The membranes 806A and 806B may be porous, elastic, or a combination thereof. Positive and/or negative pressurized media may be applied via operating channels 802 to create a pressure differential to thereby cause the membranes 806A, 806B to expand and contract along their respective planes in parallel.

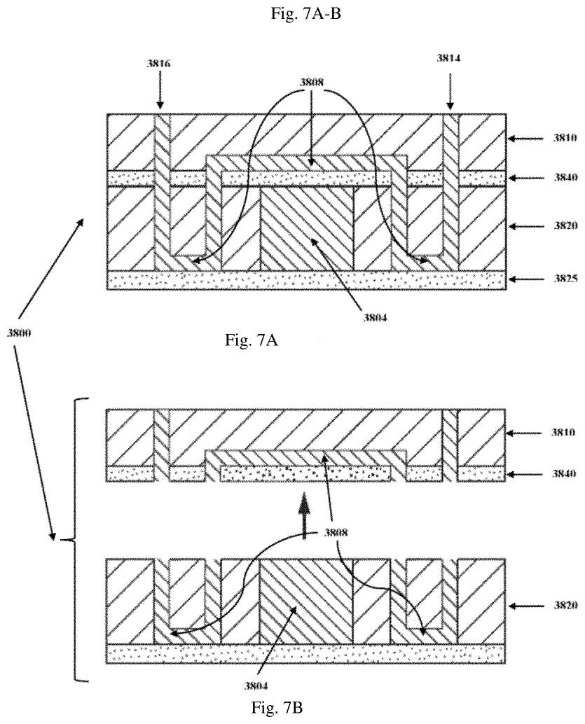

FIG. 50B illustrates a perspective view of an organ mimic device in accordance with an embodiment. As shown in FIG. 7B, the tissue interface device 900 includes operating microchannels 902A, 902B and a central microchannel 904 positioned between the microchannels 902. The central microchannel 904 includes multiple membranes 906A, 906B positioned along respective parallel x-y planes. Additionally, a wall 910 separates the central microchannel into two distinct central microchannels, having respective sections, whereby the wall 910 along with membranes 904A and 904B define microchannels 904A, 904B, 904C, and 904D. The membranes 906A and 906B at least partially porous, elastic or a combination thereof.

The device in FIG. 50B differs from that in FIG. 50A in that the operating microchannels 902A and 902B are separated by a wall 908, whereby separate pressures applied to the microchannels 902A and 902B cause their respective membranes 904A and 904B to expand or contract. In particular, a positive and/or negative pressure may be applied via operating microchannels 902A to cause the membrane 906A to expand and contract along its plane while a different positive and/or negative pressure is applied via operating microchannels 902B to cause the membrane 906B to expand and contract along its plane at a different frequency and/or magnitude. Of course, one set of operating microchannels may experience the pressure while the other set does not experience a pressure, thereby only causing one membrane to actuate. It should be noted that although two membranes are shown in the devices 800 and 900, more than two membranes are contemplated and can be configured in the devices.

In an example, shown in FIG. 50C, the device containing three channels described in FIG. 50A has two membranes 806A and 806B which are coated to determine cell behavior. In particular, membrane 806A is coated with a lymphatic endothelium on its upper surface 805A and with stromal cells on its lower surface, and stromal cells are also coated on the upper surface of the second porous membrane 805B and a vascular endothelium on its bottom surface 805C. Cells are placed in the central microchannel surrounded on top and bottom by layers of stromal cells on the surfaces of the upper and lower membranes in section 804B. Fluid such as cell culture medium or blood enters the vascular channel in section 804 C. Fluid such as cell culture medium or lymph enters the lymphatic channel in section 804A. This configuration of the device 800 allows researchers to mimic and study cell growth and invasion into blood and lymphatic vessels during immune cell migration. In the example, one or more of the membranes 806A, 806B may expand/contract in response to pressure through the operating microchannels. Additionally or alternatively, the membranes may not actuate, but may be porous or have grooves to allow cells to pass through the membranes.

In some embodiments topside is referring to an upper surface of a membrane. In some embodiments, bottom side is referring to a lower surface of a membrane.

S-1 Chip with a Double Membrane

FIG. 50 illustrates one embodiment of a double membrane microfluidic device that may find use with the present invention. In one embodiment, such a device may be used as a Lymph Node-Chip.

FIG. 50A illustrates a perspective view of an organ mimic device in accordance with an embodiment that contains three parallel microchannels separated by two porous membranes.

FIG. 50B illustrates a perspective view of an organ mimic device in accordance with an embodiment.

FIG. 50C illustrates a device containing three channels as described in FIG. 50A.

### III. Open Top Microfluidic Chips.

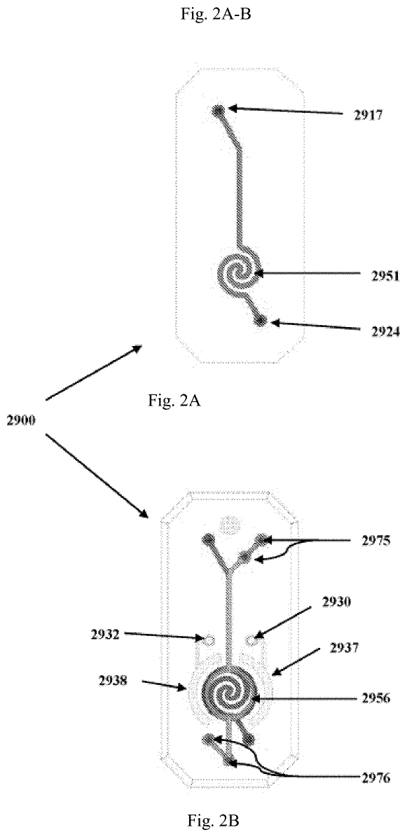

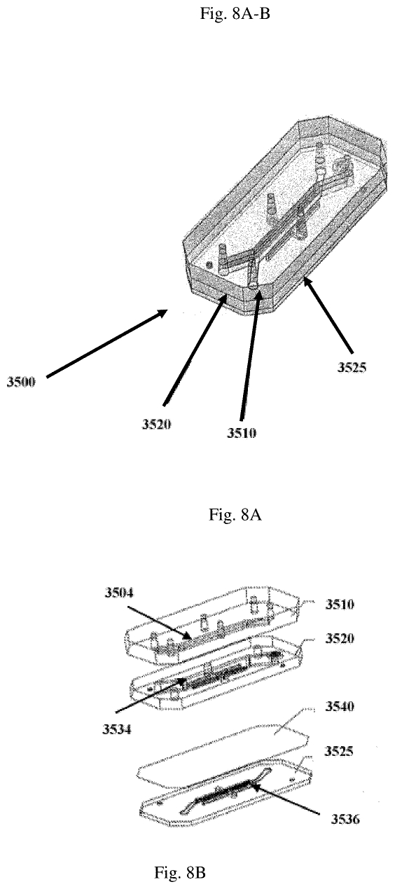

The present disclosure relates to skin-on-chips, such as fluidic devices comprising one or more cells types for the simulation one or more of the function of skin components. Accordingly, the present disclosure additionally describes open-top skin-on-chips, see, e.g. schematics in FIG. 2A-B through FIG. 8A-B. U.S. Pat. No. 8,647,861, Organ mimic device with microchannels and methods of use and manufacturing thereof, herein incorporated by reference in its entirety.

### Stretchable Open Top Chips

In one embodiment, the present invention contemplates a stretchable open top chip device 2900 comprising at least one spiral microchannel 2951 configured with at least one fluid inlet 2917 and at least one fluid outlet 2924. FIG. 2A. In one embodiment, the microfluidic chip device 2900 further comprises a upper microchannel with a circular chamber 2956 configured with a first fluid or gas port pair 2975 and second fluid or gas port pair 2976, a first vacuum port 2930 connected to a first vacuum chamber 2937 and a second vacuum port 2932 connected to a second vacuum chamber 2938, wherein the vacuum chambers are proximally configured around the spiral microchannel. In one embodiment, the upper microchannel with a circular chamber 2956 is positioned above the spiral microchannel 2951. FIG. 2B.

Although it is not necessary to understand the mechanism of an invention it is believed that the stretchable open top chip design represents a fundamental shift in architecture as compared to conventional “tissue-on-a-chip” designs. It is further believed that the open top design is compatible with 3D scaffold models. For example, an open top chip design may include, but is not limited to, three layers exemplified by a bottom channel, a middle chamber and a top channel. In one embodiment, the bottom channel layout may be spiral in shape in order to fit within the circular shape of the chamber. In another embodiment, the top channel allows for the ability to run media solutions or humidity-controlled gases (e.g., for example, air and/or oxygen-carbon dioxide mixtures such as 95% 0 j5% CO₂) to prevent gel evaporation. In another embodiment, the membrane is porous to facilitate cell-to-cell communication. Other embodiments provide a vacuum channel design that provides a mechanical stretch to the entire 3D scaffold thickness.

Furthermore, the open top stretchable chips as contemplated herein are useful for biological interfaces, co-cultures, multiple cell type cultures, tissue stretching, 3D scaffold models, micro-patterning and tissue chips including, but not limited to, skin, lung and intestine (e.g., gut). In one embodiment, an open top stretchable device may have the following specifications:

| Body Material | PDMS Sylgard 184 | |
|---|---|---|
| Membrane Material | PDMS Sylgard 184 | |
| Dimensions | | |
| Width | 15.87 | mm |
| Length | 35.87 | mm |
| Height | 6.0 | mm |
| Top Channel Dimensions | | |
| Top Channel Height | 200 | μm |
| Top Channel Diameter | 5.70 | mm |
| Top Chamber Dimensions | 5.70 | mm |
| Top Chamber Diameter | 4.00 | mm |
| Top Chamber Height | 102.7 | mm² |
| Top Channel Volume | 25.52 | mm² |
| Bottom Channel Dimensions | | |
| Bottom Channel Width | 600 | μm |

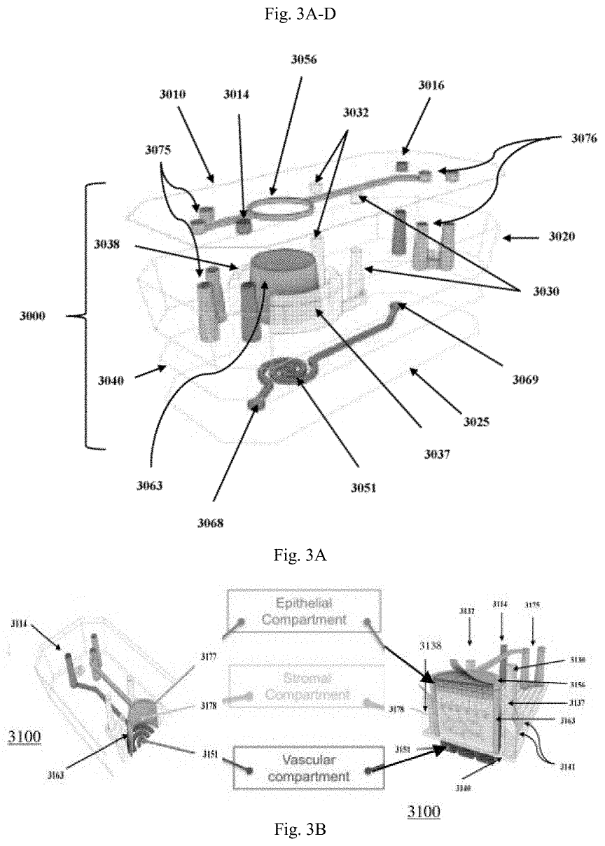

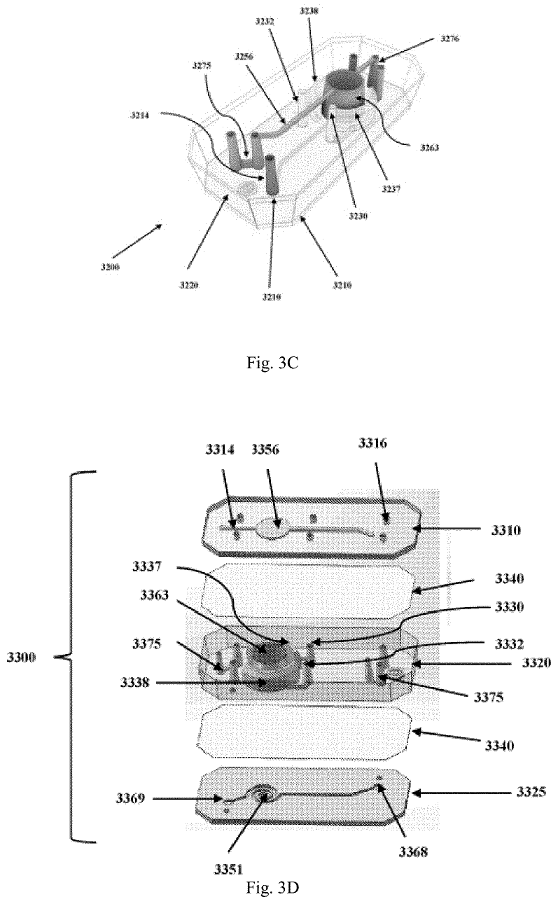

In one embodiment, the present invention contemplates a stretchable open top chip device 3000 comprising: i) a fluidic cover 3010 comprising an upper microchannel with a circular chamber 3056 configured with a first fluid or gas port pair 3075 and second fluid or gas port pair 3076; a fluid inlet port 3014, a fluid outlet port 3016, a first vacuum port 3030 and a second vacuum port 3032; ii) a top structure 3020 comprising a chamber 3063, a first vacuum chamber 3037 connected to the first vacuum port 3030, and a second vacuum chamber 3038, connected to the second vacuum port 3032, wherein the upper microchannel with a circular chamber 3056 overlays the top surface of the chamber 3063; and iii) a bottom structure 3025 comprising a spiral microchannel 3051 comprising an inlet well 3068 connected to the fluid inlet port 3014 and an outlet well 3069 connected to the fluid outlet port 3016, wherein a membrane 3040 is layered between the top structure 3020 and bottom structure 3025. FIG. 3A.

In one embodiment, the present invention contemplates a stretchable open top chip device 3100 comprising a chamber 3163 comprising an epithelial region 3177 and a dermal region 3178. In one embodiment, the epithelial region comprises an epithelial cell layer. In one embodiment, the dermal region comprises a dermal cell layer, wherein said epithelial cell layer adheres to the surface of the dermal cell layer. In one embodiment, the device further comprises a spiral microchannel 3151 in fluid communication with a fluid inlet port 3114, wherein the microchannel comprises a plurality of vascular cells. In one embodiment, a membrane 3140 is placed between the chamber dermal cell layer and the microchannel plurality of vascular cells. In one embodiment, the device further comprises an upper microchannel with a circular chamber 3156 connected to a fluid or gas port pair 3175. In one embodiment, the device further comprises a first vacuum port 3130 connected to a first vacuum chamber 3137 and a second vacuum port 3132 connected to a second vacuum chamber 3138. In one embodiment, the membrane 3140 comprises a PDMS membrane comprising a plurality of pores 3141, wherein said pores 3141 are approximately 50 μm thick, approximately 7 μm in diameter, packed as 40 μm hexagons, wherein each pore has a surface area of approximately 0.32 cm². Although it is not necessary to understand the mechanism of an invention, it is believed that the pore surface area contacts a gel layer (if present). FIGS. 3A and 3B.

In one embodiment, the present invention contemplates a stretchable open top chip device 3200 comprising: i) a fluidic cover 3210 comprising an upper microchannel with a circular chamber 3256 configured with a first fluid or gas port pair 3275 and second fluid or gas port pair 3276; a fluid inlet port 3214, a fluid outlet port 3216, a first vacuum port 3230 and a second vacuum port 3232; ii) a top structure 3220 comprising a chamber 3263, a first vacuum chamber 3237 connected to the first vacuum port 3230, and a second vacuum chamber 3238, connected to the second vacuum port 3232, wherein the upper microchannel with a circular chamber 3256 seals with the top surface of the chamber 3263; and iii) a bottom structure 3225 layered underneath said top structure 3220. FIG. 3C.

FIGS. 3A and 3B illustrate exploded views of two embodiments of a stretchable open top chip device comprising: i) a fluidic cover 3310 comprising an upper microchannel with a circular chamber 3356 configured with a first fluid or gas port pair 3375 and second fluid or gas port pair 3376; a fluid inlet port 33 14, a fluid outlet port 3316, a first vacuum port 3330 and a second vacuum port 3332; ii) a top structure 3320 comprising a chamber 3363, a first vacuum chamber 3337 connected to the first vacuum port 3330, and a second vacuum chamber 3338, connected to the second vacuum port 3332, wherein the upper microchannel with a circular chamber 3356 overlays the top surface of the chamber 3363 and a first membrane 3340 layered between the fluidic cover 3310 and the top structure 3320; and iii) a bottom structure 3325 layered underneath said top structure 3220, wherein a second membrane 3340 is layered between the bottom structure 3325 and the top structure 3320. FIG. 3D.

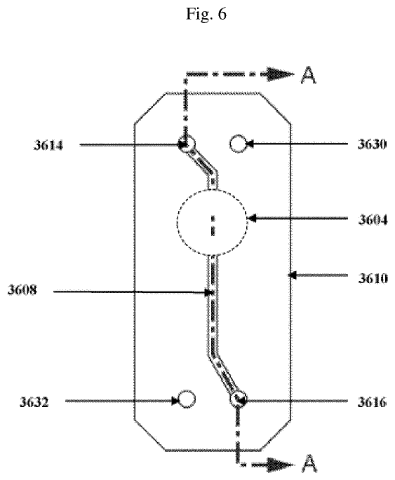

FIG. 6. In one embodiment, the present invention contemplates a fully assembled stretchable open top microfluidic device 3600 comprising a fluidic cover 3610 comprising microfluidic channel 3608, a first vacuum port 3630 and a second vacuum port 3632, wherein the microfluidic channel 3608 terminates at either end at an inlet port 3614 and an outlet port 3616, respectively.

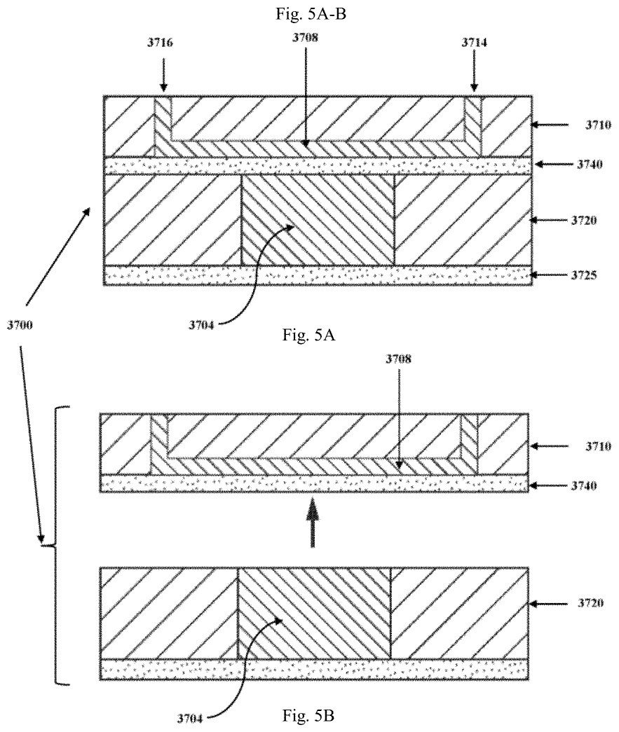

A first cross-sectional view across plane A of FIG. 6 presents an open top microfluidic device 3700 FIG. 5A-B in an assembled configuration comprising a fluidic cover 3710 attached to a membrane 3740, wherein the membrane 3740 overlays an open region 3704 (shown as hidden open region 3604 in FIG. 6) within a top structure 3720 that is attached to a bottom structure 3725. FIG. 5A. A second cross-section view across plane A of FIG. 6 presents an open top microfluidic device 3700 in a separated configuration where a fluidic top 3710 comprising a membrane 3740 is removed from top structure 3720 thereby providing access to an open region 3704, wherein a microfluidic channel 3608 is configured within the fluidic cover 3710. FIG. 5B.

A third cross-sectional view across plane A of FIG. 6 presents an open top microfluidic device 3800 in an assembled configuration comprising a fluidic cover 3810 attached to a membrane 3840, wherein the membrane 3840 overlays an open region 3804 (shown as hidden open region 3604 in FIG. 6) within a top structure 3820 that is attached to a bottom structure 3825. FIG. 7A. A fourth cross-section view across plane A of FIG. 6 presents an open top microfluidic device 3800 in a separated configuration where a fluidic top 3810 comprising a membrane 3840 is removed from top structure 3820 thereby providing access to an open region 3804, wherein a microfluidic channel 3608 is configured to traverse between fluidic cover 3810 and top structure 3820. FIG. 7B.

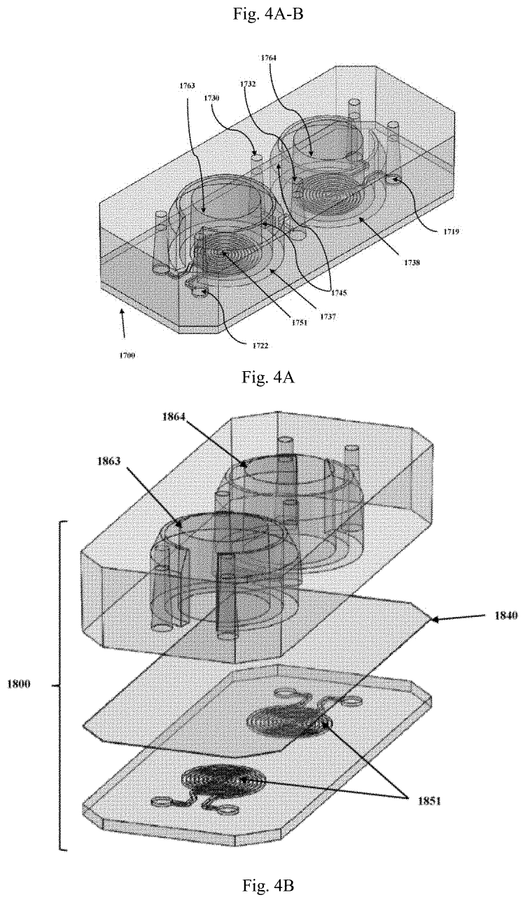

FIG. 4B. shows an exemplary exploded view of one embodiment of an open-top chip device 1800, wherein a membrane 1840 resides between the bottom surface of the first chamber 1863 and the second chamber 1864 and the at least two spiral microchannels 1851. Open top microfluidic chips include but are not limited to chips having removable covers, such as removable plastic covers, paraffin covers, tape covers, etc.

In some embodiments of a microfluidic device, it is desirable to include a cover that comprises sensors or actuators. For example, a cover can comprise one or more electrodes that can be used for measurement of electrical excitation. In some embodiments, such as where the device comprises a membrane (e.g., membrane 540), the one or more electrodes can be used to perform a measurement of trans-epithelial electrical resistance (TEER) for the membrane. It may also be desirable to include one or more electrodes on the opposite side of the membrane 540. In some embodiments, the electrodes can be included in a bottom structure (e.g., bottom structure 525). In some embodiments, the bottom structure can be an open bottom with bottom electrodes included on a bottom cover that can be brought into contact with the bottom structure. The bottom cover may support any of the features or variations discussed herein in the context of a top cover, including, for example, removability, fluidic channels, multiple layers, clamping features, etc.

FIG. 3B shows exemplary schematic views of one embodiment of an open-top chip device in relation to exemplary cell compartments, e.g. epithelial, stromal and vascular. In one embodiment, the present invention contemplates a stretchable open top chip device 3100 comprising a chamber 3163 comprising an epithelial region 3177 and a dermal region 3178. In one embodiment, the epithelial region comprises an epithelial cell layer. In one embodiment, the dermal region comprises a dermal cell layer, wherein said epithelial cell layer adheres to the surface of the dermal cell layer. In one embodiment, the device further comprises a spiral microchannel 3151 in fluid communication with a fluid inlet port 3114, wherein the microchannel comprises a plurality of vascular cells, in one embodiment, a membrane 3140 is placed between the chamber dermal cell layer and the microchannel plurality of vascular cells. In one embodiment, the device further comprises an upper microchannel with a circular chamber 3156 connected to a fluid or gas port pair 3175. In one embodiment, the device further comprises a first vacuum port 3130 connected to a first vacuum chamber 3137 and a second vacuum port 3132 connected to a second vacuum chamber 3138. In one embodiment, the membrane 3140 comprises a PDMS membrane comprising a plurality of pores 3141, wherein said pores 3141 are approximately 50 μm thick, approximately 7 μm in diameter, packed as 40 μm hexagons, wherein each pore has a surface area of approximately 0.32 cm². Although it is not necessary to understand the mechanism of an invention, it is believed that the pore surface area contacts a gel layer (if present). FIGS. 3A and 3B.

FIG. 3C shows another exemplary schematic of an open top microfluidic chip showing embodiments of a stretchable open top chip device 3200. In one embodiment, the present invention contemplates a stretchable open top chip device 3200 comprising: i) a fluidic cover 3210 comprising an upper microchannel with a circular chamber 3256 configured with a first fluid or gas port pair 3275 and second fluid or gas port pair 3276; a fluid inlet port 3214, a fluid outlet port 3216, a first vacuum port 3230 and a second vacuum port 3232; ii) a top structure 3220 comprising a chamber 3263, a first vacuum chamber 3237 connected to the first vacuum port 3230, and a second vacuum chamber 3238, connected to the second vacuum port 3232, wherein the upper microchannel with a circular chamber 3256 seals with the top surface of the chamber 3263; and iii) a bottom structure 3225 layered underneath said top structure 3220. FIG. 3C.

Many of the problems associated with earlier systems can be solved by providing an open-top style microfluidic device that allows topical access to one or more parts of the device or cells that it comprises. For example, the microfluidic device can include a removable cover, that when removed, provides access to the cells of interest in the microfluidic device. In some aspects, the microfluidic devices include systems that constrain fluids, cells, or biological components to desired area(s). The improved systems provide for more versatile experimentation when using microfluidic devices, including improved application of treatments being tested, improved seeding of additional cells, and/or improved aerosol delivery for select tissue types.

It is also desirable in some aspects to provide access to regions of a cell-culture device. For example, it can be desirable to provide topical access to cells to (i) apply topical treatments with particulate matter, e.g. pigments, such as used in tattoo inks, liquid, such as pigment diluents used with tattoo inks, gaseous, solid, semi-solid, or aerosolized reagents, (ii) apply a tattoo, e.g. access for using a tattoo gun and a tattoo needle for wounding, for injecting pigments, etc., (iii) obtain samples and biopsies, or (vi) add additional cells or biological/chemical components.

Therefore, the present disclosure relates to fluidic systems that include a fluidic device, such as a microfluidic device with an opening that provides direct access to device regions or components (e.g. access to the gel region, access to one or more cellular components, etc.). Although the present disclosure provides an embodiment wherein the opening is at the top of the device (referred to herein with the term “open top”), the present invention contemplates other embodiments where the opening is in another position on the device. For example, in one embodiment, the opening is on the bottom of the device. In another embodiment, the opening is on one or more of the sides of the device. In another embodiment, there is a combination of openings (e.g. top and sides, top and bottom, bottom and side, etc.).

While detailed discussion of the “open top” embodiment is provided herein, those of ordinary skill in the art will appreciate that many aspects of the “open top” embodiment apply similarly to open bottom embodiments, as well as open side embodiments or embodiments with openings in any other regions or directions, or combinations thereof. Similarly, the device need not remain “open” throughout its use; rather, as several embodiments described herein illustrate, the device may further comprise a cover or seal, which may be affixed reversibly or irreversibly. For example, removal of a removable cover creates an opening, while placement of the cover back on the device closes the device. The opening, and in particular the opening at the top, provides a number of advantages, for example, allowing (i) the creation of one or more gel layers for simulating the application of topical treatments on the cells, tissues, or organs, or (ii) the addition of chemical or biological components such as the seeding of additional cell types for simulated tissue and organ systems. The present disclosure further relates to improvement in fluidic system(s) that improve the delivery of topicals, such as pigments, pigment diluents, such as used with tattoo inks, to simulated tissue and organ systems, such as simulated skin-tissues.

The present invention contemplates a variety of uses for these open top microfluidic devices and methods described herein. In one embodiment, the present invention contemplates a method of topically testing an agent (whether a drug, food, gas, or other substance) comprising 1) providing a) an agent and b) microfluidic device comprising i) a chamber, said chamber comprising a lumen and projections into the lumen, said lumen comprising ii) a gel matrix anchored by said projections and comprising cell in, on or under said gel matrix, said gel matrix positioned above iii) a porous membrane and under iv) a removable cover, said membrane in contact with v) fluidic channels; 2) removing said removable cover; and 3) topically contacting said cells in, on or under said gel matrix with said agent. In one embodiment, said agent is in an aerosol. In one embodiment, agent is in a liquid, gas, gel, semi-solid, solid, or particulate form. These uses may apply to the open top microfluidic chips described below and herein.

In one embodiment, the present invention contemplates an open-top chip device 1700 comprising: i) a first chamber 1763 and a second chamber 1764, wherein each chamber is surrounded by a deformable surface 1745; and ii) at least two spiral microchannels 1751 located on the bottom surface of the chambers, wherein each of the microchannels are in fluidic communication with an inlet port 1719 and an outlet port 1722 and are respectively configured with a first vacuum port 1730 or a second vacuum port 1732, such that each vacuum port is respectively connected to a first vacuum chamber 1737 or a second vacuum chamber 1738. FIG. 4A. An exploded view of the embodiment depicted FIG. 4B shows an open-top chip device 1800, wherein a membrane 1840 resides between the bottom surface of the first chamber 1863 and the second chamber 1864 and the at least two spiral microchannels 1851. FIG. 4B.

As another example, the use of an open-top chip allows electrical stimulation, e.g. using electrodes, and allows recording electrical measurements in real-time, e.g. recording TEER, e.g. epithelial layer, etc.

Additional embodiments of an open top chip.

In one embodiment, the present invention contemplates a tall channel stretchable open top chip device 3500 comprising: i) a fluidic cover 3510 comprising an open region 3504; ii) a top structure 3520 comprising an upper microchannel 3534 attached to the fluidic cover 3510; iii) a bottom structure 3525 comprising a lower microchannel 3536 attached to the top structure 3520; and iv) a membrane 3540 layer between the bottom structure 3525 and the top structure 3520. In one embodiment, the open region 3504, upper microchannel 3534 and lower microchannel 3536 are configured to at least partially overlay each other. FIG. 8A and FIG. 8B. Although not intended to be limiting, the tall channel stretchable open top chip device 3500 may also comprise a vacuum port pair and/or inlet/outlet ports as shown and described above.

A. Open Top Microfluidic Chips without Gels.

In one embodiment, open top organ-on-chips do not contain gels, either as a bulk gel or a gel layer. Thus, the present invention also contemplates, in one embodiment, a layered structure comprising i) fluidic channels covered by ii) a porous membrane, said membrane comprising iii) a layer of cells and said membrane positioned below said cells. In one embodiment, there is a removable cover over the cells.

Additional embodiments are described herein that may be incorporated into open top chips without gels.

B. Open Top Microfluidic Chips with Gels.

Furthermore, the present disclosure contemplates improvements to fluidic systems that include a fluidic device, such as a microfluidic device with an open-top region that reduces the impact of stress that can cause the delamination of tissue or related component(s) (e.g., such as a gel layer). Thus, in a preferred embodiment, the open-top microfluidic device comprises a gel matrix. In one embodiment, the open-top microfluidic device does not contain a bulk gel.

The present invention also contemplates, in one embodiment, a layered structure comprising i) fluidic channels covered by ii) a porous membrane, said membrane comprising iii) a layer of cells and said membrane positioned below iv) a gel matrix. In one embodiment, there is a removable cover over the gel matrix (and/or cells). It is not intended that the present invention be limited to embodiments with only one gel or gel layer. In one embodiment, the layered structure further comprises a second gel matrix (e.g. positioned under said membrane). The gel(s) or coatings can be patterned or not patterned. Moreover, when patterned, the pattern need not extend to the entire surface. For example, in one embodiment, at least a portion of said gel matrix is patterned. It is not intended that the present invention be limited by the nature or components of the gel matrix or gel coating. In one embodiment, gel matrix comprises collagen. A variety of thickness is contemplated. In one embodiment of the layered structure, said gel matrix is between 0.2 and 6 mm in thickness.

Also described is a simulated lumen further comprising gel projections into the simulated lumen. Thus, in yet another embodiment, the present invention contemplates a microfluidic device comprising i) a chamber, said chamber comprising a lumen and projections in the lumen, said lumen comprising ii) a gel matrix anchored by said projections, said gel matrix positioned above iii) a porous membrane, said membrane in contact with iv) fluidic channels. In one embodiment, said membrane comprises cells. The projections serve as anchors for the gel. The projections, in one embodiment, project outward from the sidewalls. The projections, in another embodiment, project upward. The projects, in another embodiment, project downward. The projections can take a number of forms (e.g. a T structure, a Y structure, a structure with straight or curving edges, etc.). In some embodiments, there are two or more projections; in other embodiments, there are four or more projections to anchor the gel matrix. In one embodiment, the membrane is above said fluidic channels.

In other embodiments, open top microfluidic chips comprise partial lumens as described herein for closed top chips. Thus, in some embodiments, open top microfluidic chips comprise lumens formed by viscous fingering described herein for closed top chips.

Lumen gel structures may be used in several types of embodiments for open top microfluidic chips, e.g. epithelial cells or parenchymal cells can be attached to outside of the gel, or within the gel. In some embodiments, LPDCs may be added within the gel, below the gel, or above the gel. In some embodiments, stomal cells are added within the gel. In some embodiments, stomal cells are attached to the side of the gel opposite from the lumen. In some embodiments, endothelial cells are located below the gel on the side opposite the lumen. In some embodiments, endothelial cells may be present within the gel.

Additional embodiments are described herein that may be incorporated into open top chips with gels, with or without gels.

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

### VI. Chip Activation.

A. Chip Activation Compounds (ER1).

In one embodiment, bifunctional crosslinkers are used to attach one or more extracellular matrix (ECM) proteins. Buffers are referred to as ER2. A variety of such crosslinkers are available commercially, including (but not limited to) the following compounds:

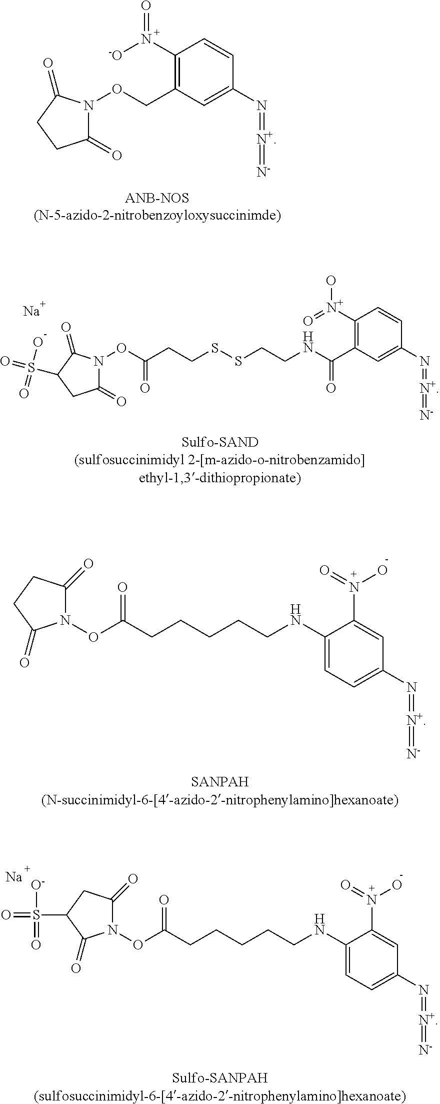

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (—NH₂) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. Tris and glycine

will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

B. Exemplary methods of Chip Activation.

### Prepare and Sanitize Hood-Working Space:

1. S-1 Chip Handling—Use aseptic technique, hold Chip using Carrier
a. Use 70% ethanol spray and wipe the exterior of Chip package prior to bringing into hood.
b. Open package inside hood
c. Remove Chip and place in sterile petri dish (6 Chips/Dish).
d. Label Chips and Dish with respective condition and Lot #.
   2. Surface Activation with Chip Activation Compound (light and time sensitive)
a. Turn off light in biosafety hood.
b. Allow vial of Chip Activation Compound powder to fully equilibrate to ambient temperature (to prevent condensation inside the storage container, as reagent is moisture sensitive).
c. Reconstitute the Chip Activation Compound powder with ER-2 solution.
   i. Add 10 ml Buffer, such as HEPES, into a 15 ml conical covered with foil.
   ii. Take 1 ml Buffer from above conical and add to chip Activation Compound (5 mg) bottle, pipette up and down to mix thoroughly and transfer to same conical.
   iii. Repeat 3-5 times until chip Activation Compound is fully mixed.
   iv. NOTE: Chip Activation Compound is single use only, discard immediately after finishing Chip activation, solution cannot be reused.
d. Wash channels.
   i. Inject 200 ul of 70% ethanol into each channel and aspirate to remove all fluid from both channels
   ii. Inject 200 ul of Cell Culture Grade Water into each channel and aspirate to remove all fluid from both channels
   iiii. Inject 200 ul of Buffer into each channel and aspirate to remove fluid from both channels
e. Inject Chip Activation Compound Solution (in buffer) in both channels
   i. Use a P200 and pipette 200 ul to inject Chip Activation Compound/Buffer into each channel of each chip (200 ul should fill about 3 Chips (Both Channels))
   ii. Inspect channels by eye to be sure no bubbles are present. If bubbles are present, flush channel with Chip Activation Compound/Buffer until bubbles have been removed
f. UV light activation of Chip Activation Compound: Place Chips into UV light box
   i. UV light treat Chips for 20 min
   ii. While the Chips are being treated, prepare ECM Solution.
   iii. After UV treatment, gently aspirate Chip Activation Compound/Buffer from channels via same ports until channels are free of solution
   iv. Carefully wash with 200 ul of Buffer solution through both channels and aspirate to remove all fluid from both channels
   v. Carefully wash with 200 ul of sterile DPBS through both channels
   vi. Carefully aspirate PBS from channels and move on to: ECM-to-Chip.

### V. Exemplary Devices for Simulating a Function of a Tissue.

Some embodiments described herein relate to devices for simulating a function of a tissue, in particular a gastrointestinal tissue. In one embodiment, the device generally comprises (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber. The first side of the membrane may have an extracellular matrix composition disposed thereon, wherein the extracellular matrix (ECM) composition comprises an ECM coating layer. In some embodiments, an ECM gel layer e.g. ECM overlay, is located over the ECM coating layer.

### VI. Ecm Coating.

To determine optimum conditions for cell attachment, the surface-treated material (e.g., APTES-treated or plasma-treated PDMS) can be coated with an ECM coating of different extracellular matrix molecules at varying concentrations (based on the resulting cell morphology and attachment).

### VII. ECM Overlay.

The ECM overlay is typically a “molecular coating,” meaning that it is done at a concentration that does not create a bulk gel. In some embodiments, an ECM overlay is used. In some embodiments, an ECM overlay is left in place throughout the co-culturing. In some embodiments, an ECM overlay is removed, e.g. when before seeding additional cells into a microfluidic device. In some embodiments, the ECM layer is provided by the cells seeded into the microfluidic device.

Although cells described for use in a Skin-Chip make their own ECM, it is contemplated that ECM in predisease and diseased states may be found in areas around sites of cell growth. Further, the protein microenvironment provided by ECM also affects cells. Thus it is contemplated that tissue-derived ECM may carry over a disease state. Therefore, in addition to the ECM described herein, ECM used in microfluidic devises of the present inventions may be derived from or associated with areas in and around sites of cells. In one embodiment, a device comprising tissue-derived ECM may be used as described herein, to identity contributions to healthy or disease states affected by native ECM.

For example, ECM may be isolated from biopsies of healthy, non-disease and disease areas as tissue-derived ECM. Isolates for use may include cells within or attached or further processed to remove embedded cells for use in the absence of the cells.

Additional examples of ECM materials include but are not limited to Matrigel®, Cultrex®, ECM harvested from humans, etc.

### EXPERIMENTAL

The following are nonlimiting exemplary readouts:

Histological evaluation: e.g. Hematoxylin and Eosin (H&E) histochemical staining and Immunostaining, for determining the presence of at least four layers characteristic of mature epidermis: basal, spinosum, granulosum, and corneum. In general for a healthy in vivo skin model, as one example, the epidermis should have a full-thickness of 8-12 cell layers; including a stratum basal layer should have highly compact basal cells that are aligned perpendicular to the basement membrane.

Immunostaining (IF):Biomarkers characteristic of the stratified epidermal layers should include:
Basement membrane markers: collagen IV, Laminin 5
Stratum basal: Keratin 14
Stratum spinosum: Keratin 10
Stratum granulosum: Filaggrin
Stratum corneum: Involucrin, Loricrin
   Quality control of tissue reproducibility from sample to sample and lot to lot can also be established by comparison of ET50 values to a baseline value. Example provided from MatTek for their baseline ET50 value on EpiDerm200—average 6.82 h. Exposure to 1.0% Triton X-100 topically over 12 h.
   ET50 determined by MTT or Presto Blue assay (ET50 time of exposure leading to 50% loss in viability of tissue compared to control)
   shows an exemplary quantitative Assessment of Functional Response (Triton X-100)—QC

TABLE 20.3
EpiDerm-200 Triton X-100 ET50 Database Summary

| | EPI-200 | EPI-200 | | |
|---|---|---|---|---|
| Year | Triton ET-50 | Triton C.V. | Lots | Avg. C.V. |
| 2000^{a} | 6.76 | 16.4 | 88 | 6.2 |
| 1999 | 6.75 | 18.2 | 146 | 5.7 |
| 1998 | 7.24 | 17.9 | 175 | 9.2 |
| 1997 | 6.78 | 15.9 | 228 | 9.9 |
| 1996 | 6.74 | 14.6 | 184 | 9.6 |
| 1995 | 6.65 | 77.8 | 112 | 4.9 |

^{a}Through September 2000.

Barrier Function: Permeability of Cascade Blue (3000 MW); Quantification of skin permeability to Cascade Blue by topical deposition of 50 μl of 10 μM Cascade Blue, followed by 1 hour static incubation. Bottom channel medium collected and Papp determined by quantification of Cascade Blue present in the sampled medium using a plate-reader. Papp values should be between 2 to 3×10\hat{ }6 cm/s.
Permeability of Testosterone and Caffeine; Requires mass spectrometer for detection. These values can be correlated to in vivo skin.
Topical Compound Test for Toxicity, Corrosivity, Irritancy, Phototoxicity viability assays performed are MTT or Presto Blue. Corrosivity: a material is considered corrosive if cell viability is <50% after 3 min exposure or <15% after 60 min exposure.
Phototoxicity: a material is considered phototoxic if cell viability is <30% after exposure to 6 J/cm2 UVA (material must be applied topically overnight prior to UV exposure); positive control 0.001% Chloropromazine.

### Irritancy:

Method 1: ET50 values determined by MTT or Presto Blue assay will define the class of irritancy based on in vivo classification (see table below). Topical exposures will include 3 exposure times of 2 h, 5 h and 18 h to 100 ul or 100 mg of a test material. Positive control include 5% SDS and 1% Triton X-100.
Method 2: a material is considered an irritant is viability is <50% after 1 h topical exposure to 30 ul or 25 mg/of test material (OECD TG 439)
Method 3: a material is considered an irritant if there is an increase in the following cytokines compared to control by ELISA is indicative of skin irritant—IL-1alpha, IL-8, prostaglandin PGE2. MatTek Irritancy protocol

### Example 1—Keratinocyte and Fibroblast Cell Culture

This example describes the preparation of keratinocytes, and in particular human foreskin keratinocytes (HFKs). An aliquot of Lonza Gold KGM media (Lonza 192060) is placed in a 50 ml tube (i.e. with 1 cryovial of HFK cells, one needs 12 ml for the flask, 10 ml for the washing step and 1 to 5 ml to break the pellet for a total of about 25 ml). The medium is warmed by putting it into the water bath for 5-10 min and then transferred inside the sterile hood. The 15 and 50 ml conical tubes are prepared as needed, along with flasks. These are filled with the appropriate amount of Lonza medium.

To thaw the HFKs, a cryovial is removed from the liquid nitrogen container and transferred into the basket containing dry ice. The cryovial is placed into the water bath until the freezing medium inside it is completely melted. The cryovial is sprayed with ethanol and brought to the sterile hood. The cryovial is opened in the hood and the contents are collected from the cryovial (freezing medium+ cells) using a 1000 μl pipette. The contents are transferred into the 15 ml conical tube containing Lonza Gold KGM medium previously warmed. This conical tube is closed and then tilted to mix. Thereafter, it is centrifuged at 1000 rpm for 5 minutes. The conical tube is sprayed with ethanol and returned to the sterile hood. It is opened and the supernatant is withdrawn, leaving the cell pellet. The pellet is re-suspended using fresh pre-warmed Lonza Gold KGM and the mixture is transferred to a flask (or flasks), which were previously filled with Lonza Gold KGM medium. The flasks are gently agitated to make sure that the medium covers the entire bottom surface. The flasks are then transferred to the incubator. The keratinocytes are fed with new media approximately every other day (about every 36 hours).

To thaw the fibroblasts, a cryovial is removed from the liquid nitrogen tank and transferred into the basket containing dry ice. The cryovial is placed into the water bath until the freezing medium inside it is completely melted. The cryovial is sprayed with ethanol and brought to the sterile hood. The cryovial is opened in the hood and the contents are collected from the cryovial (freezing medium+ cells) using a 1000 μl pipette. Tee contents are transferred into the 15 ml conical tube containing Lonza FGM-2 medium previously warmed. This conical tube is closed and then tilted to mix. Thereafter, it is centrifuged at 1200 rpm for 5 minutes. The conical tube is sprayed with ethanol and returned to the sterile hood. It is opened and the supernatant is withdrawn, leaving the cell pellet. The pellet is re-suspended using fresh pre-warmed Lonza FGM-2 and the mixture is transferred to a flask (or flasks) that were previously filled with Lonza FGM-2 medium. The flasks are gently agitated to make sure that the medium covers the entire bottom surface. The flasks are then transferred to the incubator. The fibroblasts are fed with new media approximately every other day (about every 36 hours).

For detaching the HFKs by trypsinization, the protocol is as follows. First, an aliquot Lonza Gold KGM (Lonza 192060), Lonza reagent subculture reagent CC-5034 and E-medium (or variants) 10% FBS medium is placed in 15 ml and 50 ml tubes. It is convenient to us 4 mls of Lonza reagent subculture reagent CC-5034 per T75 flask and to add 8 mls of 10% FBS medium to the flask (which corresponds to 2 ml for each ml of reagent Lonza reagent subculture reagent CC-5034). The media and enzymes are warmed by putting it into the water bath for 5-10 min. The flask containing HFK (typically when the cells are between 50 and 70% confluence) is removed from the incubator, sterilized on the outside with ethanol, and transferred into the hood. The flask is opened and the Lonza Gold KGM medium is aspirated, being careful to not scratch the bottom flask surface where the cells are attached. Fresh pre-warmed Lonza Gold KGM medium (e.g. 5 mls) is then added to wash the cells. This media is also aspirated carefully. Then, 4 ml of 0.05% trypsin/EDTA (Corning 25-052 CL) is added to the flask and the flask is returned to the incubator. The detaching cells can be monitored using the microscope if desired. As a rule of thumb, keratinocytes should detach in about 2-3 minutes. Longer exposure to Lonza subculture reagent CC-5034 (or 0.05 EDTA trypsin Invitrogen 25200-056) could damage keratinocytes irreversibly. When the cells detach completely, the outside of the flask is sterilized and brought to the hood. The flask is opened and 8 ml of 10% FBS E-medium (or variants) is added to the flask (2 ml for each ml of 0.05 EDTA trypsin Corning 25-052-CL). Thereafter, the contents of the flask are conveniently transferred to a 15 ml conical tube. The tube is closed and centrifuged at 1000 rpm for 5 min. The tube is then sterilized with ethanol, returned to the hood and opened. The supernatant is gently aspirated, being careful not to disturb the cell pellet. After the supernatant is removed, the pellet is re-suspended using fresh pre-warmed Lonza Gold KGM medium. The mixture is then transferred to the flask/flasks, which were previously filled with Lonza Gold KGM medium. The flasks are gently agitated to make sure that the medium covers the entire bottom surface, and they are returned to the incubator. Feeding is as stated above.

For detaching the fibroblasts by trypsinization, the protocol is as follows. An aliquot of Lonza FGM-2 medium (Lonza CC-3132), Lonza reagent subculture reagent CC-5034 and 10% FBS medium is added in 15 ml and 50 ml tubes. It is convenient to use 4 ml Lonza reagent subculture reagent CC-5034 per T75 flask and 8 ml of 10% FBS medium to the flask (which corresponds 2 ml for each ml of reagent Lonza reagent subculture reagent CC-5034). The media and enzymes are warmed by putting them into the water bath for 5-10 min. The flask containing fibroblasts (typically when the cells are between 50 and 70% confluence) is removed from the incubator, sterilized on the outside with ethanol, and transferred into the hood. The flask is opened and the media is aspirated gently, being careful to not scratch the bottom flask surface containing the cell layer. 5 ml of fresh PBS is added to wash the cells (this can be done twice). The PBS is aspirated carefully, and 4 ml of 0.05% trypsin/EDTA (Lonza CC-5012) is added and the flask is returned to the incubator. The detaching cells can be monitored using the microscope if desired. As a rule of thumb, fibroblasts should detach in about 2-3 minutes. Longer exposure could damage the cells irreversibly. When the cells detach completely, the outside of the flask is sterilized and brought to the hood. The flask is opened and 8 ml of Trypsin Neutralizing Solution (CC-5002) [2 ml for each ml of 0.05% trypsin/EDTA (Lonza CC-5002)] is added. The flask contents are transferred to a 15 ml conical tube and this tube is centrifuged at 1000 rpm for 5 min. The tube is sterilized with ethanol and returned to the hood. The supernatant is aspirated, being careful not to disturb the cell pellet. Then, the pellet is re-suspended using fresh pre-warmed Lonza FGM-2 medium and the contents are transferred to the flask/flasks, which were previously filled with Lonza FGM-2 medium. The flasks are gently agitated to make sure that the medium covers the entire bottom surface and then returned to the incubator. Feeding is as indicated above.

### Example 2—Embedding Cells in the Dermal Layer

For embedding fibroblasts into the dermal layer (e.g. gel matrix), an exemplary method/protocol is proved as follows. First, the fibroblasts are detached using the trypsinization protocol described above. However, the pellet is re-suspended in complete E-medium low calcium (0.6 mM Ca⁺⁺), supplemented with 0.5% (V/V) FBS (Invitrogen 16140071) and 2% penicillin/streptomycin (Invitrogen 15140-122) and then added back to the flasks, where they are allowed to reach 50-60% confluence. Once again, the fibroblasts are detached according to the protocol described above. Once re-suspended, they are embedded into the dermal layer. From Day 0 to Day 1-2, the cells in the dermal layer are fed using complete E-medium low calcium (0.6 mM Ca**), supplemented with 0.5% (V/V) FBS (Invitrogen 16140071) and 100 μm ascorbic acid, RM/TI transglutaminase 50 μg/ml. From Day 1-2 to Day 3-4, the cells in the dermal layer are fed using complete E-medium low calcium (1.2 mM Ca⁺⁺), supplemented with 0.5% (V/V) FBS (Invitrogen 16140071) and 100 μm ascorbic acid and RM/TI transglutaminase 50 μg/ml. From Day 14-18 on, the cells in the dermal layer are fed using complete cornification medium (1.8 mM Ca⁺⁺), supplemented with 5% (V/V) FBS (Invitrogen 16140071) and 100 μm ascorbic acid and RM/TI transglutaminase 50 μg/ml.

### Example 3—Preparing the Dermal Layer

When beginning, pipette tips are cooled by putting into refrigerator for 15-30 min (Pipettes need to be cold when working with rat-tail type I collagen in order to avoid coagulation). Both the pipette tips and the ECM matrix should stay in an icebox or other cooler during the procedure.

In order to calculate the final volume of rat-tail type I collagen mixture needed, one calculates the number of dermal equivalent cultures that are needed. This calculation is based on 12 well+3 extra (those are needed to compensate for the ECM matrix that adheres to the surface of pipette). Where 2×10⁴ neonatal or adult Human Foreskin Fibroblast per raft are employed and 12+3 rafts are prepared, one needs 15×2×10⁴=30×10⁴ fibroblasts (or 300,000 fibroblasts). To impede fibroblasts proliferation, one can irradiate the fibroblast with 70Gy.

To make 150 μl/raft×(12+3) rafts=2.25 ml. 10% 10×DMEM or variants*=0.225 ml or 225 μl. 10% reconstruction buffer+=0.225 ml or 225 μl. 80% ECM matrix=1.8 ml or 1800 μl. (1.8 ml ECM matrix×2.4×10 1N NaOH (1M))=43.2 μl 1M NaOH (1M) (NaOH makes ECM matrix to coagulate). This is put into INCUBATOR 37° C. for 2-4 Hours.

Fibroblasts may be trypsinized using 0.05% trypsin/EDTA (Corning 25-052 CL) according to protocol described above. One can then re-suspend the fibroblast pellet in the predetermined amount of 10×DMEM or variants. This is mixed with the necessary amount of reconstitution buffer. (Note: best results are obtained when fibroblasts are collected in active growth phase, which occurs when fibroblast are between 50 and 70% confluence).

100 μl ECM+fibroblast are added to each well and this is incubated (37° C. for 2 Hours). Thereafter, 100 μl of E medium is added to the top of each collagen gel. 100 μl of E medium+RM TG* is then added to the bottom of each collagen gel. This is incubated (37° C. for 12-16 Hours).

A variety of collagen containing matrices are contemplated for making an artificial derma and ECM to embed fibroblasts: Tropoelastin: Collagen I: Collagen III: Dermatan sulfate (1 mg:3 mg:3 mg:0.5 mg); Col I (3 mg/ml)/Elastin (3 mg/ml); Col I (3 mg/ml)/Elastin (1 mg/ml); Col I (10 mg/ml)/MaxGEL; Col I (3 mg/ml)/Elastin (3 mg/ml) 1:1 MaxGel; Col I (3 mg/ml)/Elastin (3 mg/ml)/Col III (3 mg/ml) 1:1:1; MaxGel; Col I (10 mg/ml)/Elastin (10 mg/ml); etc.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1-45. (canceled)

46. A method, comprising:
a) providing,
i) a device having a membrane, wherein said membrane has a first surface,
ii) a population of dermal fibroblast cells,
iii) a population of keratinocyte cells,
iv) keratinocyte differentiation medium; and
b) seeding said dermal fibroblast cells in a gel matrix, said gel matrix positioned on said first surface of said membrane and comprising collagen and a polymer formed by the copolymerization of sucrose and epichlorohydrin;
c) seeding said keratinocyte cells on top of said gel matrix after step b);
d) culturing said keratinocyte cells in said keratinocyte differentiation medium under flow conditions; and
e) culturing said cells under an air-liquid-interface.

47. The method of claim 46, wherein said polymer formed by said copolymerization is a branched, hydrophilic polysaccharide which dissolves in aqueous solutions.

48. The method of claim 46, wherein said polymer inhibits the contraction of said gel matrix.

49. The method of claim 46, wherein said polymer delays the contraction of said gel matrix for a period of time.

50. The method of claim 46, wherein said polymer delays the contraction of said gel matrix for as much as five days.

51. The method of claim 46, wherein said culturing under air-liquid-interface conditions results in a epidermal layer positioned above a dermal layer.

52. The method of claim 51, wherein at least a portion of the epidermal layer is embedded in said dermal layer.

53. The method of claim 46, wherein said gel matrix is in contact with one or more structures that hold at least a portion of the gel in position for a time period.

54. The method of claim 46, further comprising the step of stretching the gel, the membrane or both.

55. The method of claim 51, further comprising f) exposing said epidermal layer to an agent.

56. The method of claim 51, further comprising f) wounding said epidermal layer.

57-60. (canceled)
